SEQUENCE LISTING (101 sequences)

SEQ ID NO: 1 - DNA, 1211 bp, Synthetic construct
  source 1..1211: mol_type = other DNA
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccacagcctccttgggcaacctgttcagtgcgtcaccaccctctgggggaaaaactgcctcctcatatccaacccaaacctcccctgtctcagtgtaaagccattcccccttgtcctatcaagggggagtttgctgtgacattgttggtctggggtgacacatgtttgccaattcagtgcatcacggagaggcagatcttggggataaggaagtgcaggacagcatggacgtgggacatgcaggtgttgagggctctgggacactctccaagtcacagcgttcagaacagccttaaggataagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgagaatatt

SEQ ID NO: 2 - DNA, 204 bp, Synthetic construct
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 3 - DNA, 585 bp, Synthetic construct
  source 1..585: mol_type = other DNA
ggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccg

SEQ ID NO: 4 - DNA, 693 bp, Synthetic construct
  source 1..693: mol_type = other DNA
atggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccc

SEQ ID NO: 5 - protein, 518 aa, Synthetic construct
MPKKKRKVYPYDVPDYAGSRALVVIRLSRVTDATTSPERQLESCQQLCAQRGWDVVGVAEDLDVSGAVDPFDRKRRPNLARWLAFEEQPFDVIVAYRVDRLTRSIRHLQQLVHWAEDHKKLVVSATEAHFDTTTPFAAVVIALMGTVAQMELEAIKERNRSAAHFNIRAGKYRGSLPPWGYLPTRVDGEWRLVPDPVQRERILEVYHRVVDNHEPLHLVAHDLNRRGVLSPKDYFAQLQGREPQGREWSATALKRSMISEAMLGYATLNGKTVRDDDGAPLVRAEPILTREQLEALRAELVKTSRAKPAVSTPSLLLRVLFCAVCGEPAYKFAGGGRKHPRYRCRSMGFPKHCGNGTVAMAEWDAFCEEQVLDLLGDAERLEKVWVAGSDSAVELAEVNAELVDLTSLIGSPAYRAGSPQREALDARIAALAARQEELEGLEARPSGWEWRETGQRFGDWWREQDTAAKNTWLRSMNVRLTFDVRGGLTRTIDFGDLQEYEQHLRLGSVVERLHTGMS

SEQ ID NO: 6 - DNA, 38 bp, Synthetic construct
  source 1..38: mol_type = other DNA
ggcttgtcgacgacggcggtctccgtcgtcaggatcat

SEQ ID NO: 7 - DNA, 41 bp, Synthetic construct
  source 1..41: mol_type = other DNA
cggcttgtcgacgacggcggtctccgtcgtcaggatcatcc

SEQ ID NO: 8 - skipped

SEQ ID NO: 9 - DNA, 42 bp, Synthetic construct
  source 1..42: mol_type = other DNA
ccggcttgtcgacgacggcggtctccgtcgtcaggatcatcc

SEQ ID NO: 10 - DNA, 42 bp, Synthetic construct
  source 1..42: mol_type = other DNA
ggatgatcctgacgacggagaccgccgtcgtcgacaagccgg

SEQ ID NO: 11 - DNA, 48 bp, Synthetic construct
  source 1..48: mol_type = other DNA
ggtttgtaccgtacaccactgagaccgcggtggttgaccagacaaacc

SEQ ID NO: 12 - DNA, 48 bp, Synthetic construct
  source 1..48: mol_type = other DNA
ggtttgtctggtcaaccaccgcggtctcagtggtgtacggtacaaacc

SEQ ID NO: 13 - DNA, 52 bp, Synthetic construct
  source 1..52: mol_type = other DNA
gtggtttgtctggtcaaccaccgcggtctcagtggtgtacggtacaaaccca

SEQ ID NO: 14 - protein, 423 aa, Synthetic construct
MPQFGILCKTPPKVLVRQFVERFERPSGEKIALCAAELTYLCWMITHNGTAIKRATFMSYNTIISNSLSFDIVNKSLQFKYKTQKATILEASLKKLIPAWEFTIIPYYGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYSIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 15 - protein, 374 aa, Synthetic construct
MNEQAKQLVAVTRQPALNAGVGLVLAQLAEVEARQIPGSLAEARAHCLAQGAPDILLVEVENPQTLAADLAALAECCPPQMRLVLLGERGDVTLFRWLISVGVDDYYPAPLDPDALRTGLLRLLGVPLVTSLRKGRVICVVGAAGGVGTSTVAANLAMALADQHHRQVALLDLNLHHSRHPILLGSDYAPPGEQWWQATDRLDGTLLAHTAHQLGPRLFLFYDEGQELVLGAEQLVAAVNVMAEHYSTLIIDVPDLRTHGLRALLQEADVVLWLHDFSLGALRLLGQCPQGGQAQRRLLVGNHCRGKEGRVPAQELERVCGQPHAAVLPYDHGVFVRAERAGQPLIQQKSKLARALTLLAGELVGAQVTGRGRR

SEQ ID NO: 16 - protein, 432 aa, Synthetic construct
MAPKKKRKVMSQFDILCKTPPKVLVRQFVERFERPSGEKIASCAAELTYLCWMITHNGTAIKRATFMSYNTIISNSLSFDIVNKSLQFKYKTQKATILEASLKKLIPAWEFTIIPYNGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYPIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 17 - DNA, 34 bp, Synthetic construct
  source 1..34: mol_type = other DNA
gaagttcctatactttctagagaataggaacttc

SEQ ID NO: 18 - protein, 343 aa, Synthetic construct
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 19 - DNA, 34 bp, Synthetic construct
  source 1..34: mol_type = other DNA
ataacttcgtatagcatacattatacgaagttat

SEQ ID NO: 20 - DNA, 34 bp, Synthetic construct
  source 1..34: mol_type = other DNA
ataacttcgtataaagtatcctatacgaagttat

SEQ ID NO: 21 - DNA, 34 bp, Synthetic construct
  source 1..34: mol_type = other DNA
ataacttcgtataatgtatgctatacgaagttat

SEQ ID NO: 22 - DNA, 34 bp, Synthetic construct
  source 1..34: mol_type = other DNA
ataacttcgtatagcatacattatacgaacggta

SEQ ID NO: 23 - DNA, 34 bp, Synthetic construct
  source 1..34: mol_type = other DNA
taccgttcgtatagcatacattatacgaagttat

SEQ ID NO: 24 - DNA, 34 bp, Synthetic construct
  source 1..34: mol_type = other DNA
ataacttcgtataagaaaccatatacgaagttat

SEQ ID NO: 25 - DNA, 34 bp, Synthetic construct
  source 1..34: mol_type = other DNA
ataacttcgtataatgtgtactatacgaagttat

SEQ ID NO: 26 - protein, 380 aa, Synthetic construct
MIENQLSLLGDFSGVRPDDVKTAIQAAQKKGINVAENEQFKAAFEHLLNEFKKREERYSPNTLRRLESAWTCFVDWCLANHRHSLPATPDTVEAFFIERAEELHRNTLSVYRWAISRVHRVAGCPDPCLDIYVEDRLKAIARKKVREGEAVKQASPFNEQHLLKLTSLWYRSDKLLLRRNLALLAVAYESMLRASELANIRVSDMELAGDGTAILTIPITKTNHSGEPDTCILSQDVVSLLMDYTEAGKLDMSSDGFLFVGVSKHNTCIKPKKDKQTGEVLHKPITTKTVEGVFYSAWETLDLGRQGVKPFTAHSARVGAAQDLLKKGYNTLQIQQSGRWSSGAMVARYGRAILARDGAMAHSRVKTRSAPMQWGKDEKD

SEQ ID NO: 27 - DNA, 34 bp, Synthetic construct
  source 1..34: mol_type = other DNA
tcaatttccgagaatgacagttctcagaaattga

SEQ ID NO: 28 - protein, 342 aa, Synthetic construct
MSELIISGSSGGFLRNIGKEYQEAAENFMRFMNDQGAYAPNTLRDLRLVFHSWARWCHARQLAWFPISPEMAREYFLQLHDADLASTTIDKHYAMLNMLLSHCGLPPLSDDKSVSLAMRRIRREAATEKGERTGQAIPLRWDDLKLLDVLLSRSERLVDLRNRAFLFVAYNTLMRMSEISRIRVGDLDQTGDTVTLHISHTKTITTAAGLDKVLSRRTTAVLNDWLDVSGLREHPDAVLFPPIHRSNKARITTTPLTAPAMEKIFSDAWVLLNKRDATPNKGRYRTWTGHSARVGAAIDMAEKQVSMVEIMQEGTWKKPETLMRYLRRGGVSVGANSRLMDS

SEQ ID NO: 29 - DNA, 32 bp, Synthetic construct
  source 1..32: mol_type = other DNA
taactttaaataattggcattatttaaagtta

SEQ ID NO: 30 - protein, 594 aa, Trichoplusia ni
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 31 - protein, 340 aa, Rana pipiens
MPRPKEIQEQLRKKVIEIYQSGKGYKAISKALGIQRTTVRAIIHKWRRHGTVVNLPRSGRPPKITPRAQRRLIQEVTKDPTTTSKELQASLASVKVSVHASTIRKRLGKNGLHGRVPRRKPLLSKKNIKARLNFSTTHLDDPQDFWDNILWTDETKVELFGRCVSKYIWRRRNTAFHKKNIIPTVKYGGGSVMVWGCFAASGPGRLAVIKGTMNSAVYQEILKENVRPSVRVLKLKRTWVLQQDNDPKHTSKSTTEWLKKNKMKTLEWPSQSPDLNPIEMLWYDLKKAVHARKPSNVTELGQFCKDEWAKIPPGRCKSLIARYRKRLVAVVAAKGGPTSY

SEQ ID NO: 32 - protein, 636 aa, Tribolium castaneum
MMLNWLKSGKLESQSQEQSSCYLENSNCLPPTLDSTDIIGEENKAGTTSRKKRKYDEDYLNFGFTWTGDKDEPNGLCVICEQVVNNSSLNPAKLKRHLDTKHPTLKGKSEYFKRKCNELNQKKHTFERYVRDDNKNLLKASYLVSLRIAKQGEAYTIAEKLIKPCTKDLTTCVFGEKFASKVDLVPLSDTTISRRIEDMSYFCEAVLVNRLKNAKCGFTLQMDESTDVAGLAILLVFVRYIHESSFEEDMLFCKALPTQTTGEEIFNLLNAYFEKHSIPWNLCYHICTDGAKAMVGVIKGVIARIKKLVPDIKASHCCLHRHALAVKRIPNALHEVLNDAVKMINFIKSRPLNARVFALLCDDLGSLHKNLLLHTEVRWLSRGKVLTRFWELRDEIRIFFNEREFAGKLNDTSWLQNLAYIADIFSYLNEVNLSLQGPNSTIFKVNSRINSIKSKLKLWEECITKNNTECFANLNDFLETSNTALDPNLKSNILEHLNGLKNTFLEYFPPTCNNISWVENPFNECGNVDTLPIKEREQLIDIRTDTTLKSSFVPDGIGPFWIKLMDEFPEISKRAVKELMPFVTTYLCEKSFSVYVATKTKYRNRLDAEDDMRLQLTTIHPDIDNLCNNKQAQKSH

SEQ ID NO: 33 - protein, 649 aa, Oryzias latipes
MEEVCDSSAAASSTVQNQPQDQEHPWPYLREFFSLSGVNKDSFKMKCVLCLPLNKEISAFKSSPSNLRKHIERMHPNYLKNYSKLTAQKRKIGTSTHASSSKQLKVDSVFPVKHVSPVTVNKAILRYIIQGLHPFSTVDLPSFKELISTLQPGISVITRPTLRSKIAEAALIMKQKVTAAMSEVEWIATTTDCWTARRKSFIGVTAHWINPGSLERHSAALACKRLMGSHTFEVLASAMNDIHSEYEIRDKVVCTTTDSGSNFMKAFRVFGVENNDIETEARRCESDDTDSEGCGEGSDGVEFQDASRVLDQDDGFEFQLPKHQKCACHLLNLVSSVDAQKALSNEHYKKLYRSVFGKCQALWNKSSRSALAAEAVESESRLQLLRPNQTRWNSTFMAVDRILQICKEAGEGALRNICTSLEVPMFNPAEMLFLTEWANTMRPVAKVLDILQAETNTQLGWLLPSVHQLSLKLQRLHHSLRYCDPLVDALQQGIQTRFKHMFEDPEIIAAAILLPKFRTSWTNDETIIKRGMDYIRVHLEPLDHKKELANSSSDDEDFFASLKPTTHEASKELDGYLACVSDTRESLLTFPAICSLSIKTNTPLPASAACERLFSTAGLLFSPKRARLDTNNFENQLLLKLNLRFYNFE

SEQ ID NO: 34 - protein, 340 aa, Synthetic construct
MGKSKEISQDLRKKIVDLHKSGSSLGAISKRLKVPRSSVQTIVRKYKHHGTTQPSYRSGRRRYLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 35 - protein, 340 aa, Synthetic construct
MGKSKEISQDLRKRIVDLHKSGSSLGAISKRLAVPRSSVQTIVRKYKHHGTTQPSYRSGRRRYLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 36 - DNA, 74 bp, Synthetic construct
  source 1..74: mol_type = other DNA
acttaagtgtatgtaaacttccgacttcaactgtagggtacctgattctctgggcatctctgcccactaccatg

SEQ ID NO: 37 - DNA, 75 bp, Synthetic construct
  source 1..75: mol_type = other DNA
acttaagtgtatgtaaacttccgacttcaactgtaaattttccacctttttcagttttcctcgccatatttcatg

SEQ ID NO: 38 - DNA, 33 bp, Synthetic construct
  source 1..33: mol_type = other DNA
acttaagtgtatgtaaacttccgacttcaactg

SEQ ID NO: 39 - DNA, 370 bp, Synthetic construct
  source 1..370: mol_type = other DNA
cagtcaacttagtgtatgtaaacttctgacccactggaattgtgatacagtgaattataagtgaaataatctgtctgtaaacaattgttggaaaaatgacttgtgtcatgcacaaagtagatgtcctaactgacttgccaaaactattgtttgttaacaagaaatttgtggagtagttgaaaaacgagttttaatgactccaacttaagtgtatgtaaacttccgacttcaactgtaagaatggcccattcatctatagtagcacacaatatttgcatttgtgcgacagtataagggacaattatgctatcaggcatttttccaaagtgagtaatcgaagtttttatacctttgtgtgccatgtttgcta

SEQ ID NO: 40 - DNA, 369 bp, Synthetic construct
  source 1..369: mol_type = other DNA
cagtcaacttagtgtatgtaaacttctgacccactggaattgtgatacagtgaattataagtgaaataatctgtctgtaaacaattgttggaaaaatgacttgtgtcatgcacaaagtagatgtcctaactgacttgccaaaactattgtttgttaacaagaaatttgtggagtagttgaaaaacgagttttaatgactccaacttaagtgtatgtaaacttccgacttcaactgtacaagtagaccaaatatccatatacataaaagaaaaaaatagaaaaaatttctagtgacagaaaaatgacaaagaacatactgctttattactactattaagatgtttgcttccattacactcatatgagtca

SEQ ID NO: 41 - DNA, 227 bp, Synthetic construct
  source 1..227: mol_type = other DNA
ttagtgtatgtaaacttctgacccactggaattgtgatacagtgaattataagtgaaataatctgtctgtaaacaattgttggaaaaatgacttgtgtcatgcacaaagtagatgtcctaactgacttgccaaaactattgtttgttaacaagaaatttgtggagtagttgaaaaacgagttttaatgactccaacttaagtgtatgtaaacttccgacttcaactg

SEQ ID NO: 42 - DNA, 371 bp, Synthetic construct
  source 1..371: mol_type = other DNA
caacttgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactgtatatcctccccgttgcaccctcttgatgatgctgagatgaacacagatgctcactccttgagggctctaagcttatgctgacacagacacaggtgctcacttctatgaatggcctaagatttgaggacatcatgagg

SEQ ID NO: 43 - DNA, 229 bp, Synthetic construct
  source 1..229: mol_type = other DNA
ttgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactg

SEQ ID NO: 44 - DNA, 346 bp, Synthetic construct
  source 1..346: mol_type = other DNA
ttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatgtcgaggtaggcgtgtacggtgggcgcctataaaagcagagctcgtttagtgaaccgtcagatcgcctggagcaattccacaacacttttgtcttatactt

SEQ ID NO: 45 - DNA, 368 bp, Synthetic construct
  source 1..368: mol_type = other DNA
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttgcttatgtaaaccagggcgcctataaaagagtgctgattttttgagtaaacttcaattccacaacacttttgtcttataccaactttccgtaccacttcctaccctcgtaaa

SEQ ID NO: 46 - DNA, 63 bp, Synthetic construct
  source 1..63: mol_type = other DNA
  misc_structure 1..9: note = (ggcagcggc) can be added to the 5' end to improve cleavage efficiency of resulting peptide
ggcagcggcgaaggccgcggcagcctgctgacctgcggcgatgtggaagaaaacccgggcccg

SEQ ID NO: 47 - protein, 21 aa, Synthetic construct
  REGION 1..3: note = (GSG) residues can be added to the 5' end of the peptide to improve cleavage efficiency
GSGEGRGSLLTCGDVEENPGP

SEQ ID NO: 48 - DNA, 66 bp, Synthetic construct
  source 1..66: mol_type = other DNA
  misc_structure 1..9: note = (ggcagcggc) can be added to the 5' end to improve cleavage efficiency of resulting peptide
ggcagcggcgcgaccaactttagcctgctgaaacaggcgggcgatgtggaagaaaacccgggcccg

SEQ ID NO: 49 - protein, 22 aa, Synthetic construct
  REGION 1..3: note = (GSG) residues can be added to the 5' end of the peptide to improve cleavage efficiency
GSGATNFSLLKQAGDVEENPGP

SEQ ID NO: 50 - DNA, 69 bp, Synthetic construct
  source 1..69: mol_type = other DNA
  misc_structure 1..9: note = (ggcagcggc) can be added to the 5' end to improve cleavage efficiency of resulting peptide
ggcagcggccagtgcaccaactatgcgctgctgaaactggcgggcgatgtggaaagcaacccgggcccg

SEQ ID NO: 51 - protein, 23 aa, Synthetic construct
  REGION 1..3: note = (GSG) residues can be added to the 5' end of the peptide to improve cleavage efficiency
GSGQCTNYALLKLAGDVESNPGP

SEQ ID NO: 52 - DNA, 75 bp, Synthetic construct
  source 1..75: mol_type = other DNA
  misc_structure 1..9: note = (ggcagcggc) can be added to the 5' end to improve cleavage efficiency of resulting peptide
ggcagcggcgtgaaacagaccctgaactttgatctgctgaaactggcgggcgatgtggaaagcaacccgggcccg

SEQ ID NO: 53 - protein, 25 aa, Synthetic construct
  REGION 1..3: note = (GSG) residues can be added to the 5' end of the peptide to improve cleavage efficiency
GSGVKQTLNFDLLKLAGDVESNPGP

SEQ ID NO: 54 - DNA, 90 bp, Synthetic construct
  source 1..90: mol_type = other DNA
gaagcaactttgtctaccattctgtctgagggtgccacaaatttttctttgttgaagttagcaggggatgttgaacttaaccccggccca

SEQ ID NO: 55 - protein, 30 aa, Synthetic construct
EATLSTILSEGATNFSLLKLAGDVELNPGP

SEQ ID NO: 56 - DNA, 90 bp, Synthetic construct
  source 1..90: mol_type = other DNA
ttcactgattttttcaaagccgttagagactatcatgcttcttattacaaacagagacttcaacatgacgttgaaacaaaccctggccct

SEQ ID NO: 57 - protein, 30 aa, Synthetic construct
FTDFFKAVRDYHASYYKQRLQHDVETNPGP

SEQ ID NO: 58 - DNA, 90 bp, Synthetic construct
  source 1..90: mol_type = other DNA
tactttaatataatgcacagtgatgaaatggattttgccggggggaaatttttgaatcaatgtggtgatgtggaaactaacccaggccct

SEQ ID NO: 59 - protein, 30 aa, Synthetic construct
YFNIMHSDEMDFAGGKFLNQCGDVETNPGP

SEQ ID NO: 60 - DNA, 90 bp, Synthetic construct
  source 1..90: mol_type = other DNA
ccctcaattggtaatgtcgcgcggactctgacgagggcggagattgaggatgaattgattcgtgcaggaattgaatcaaatcctggacct

SEQ ID NO: 61 - protein, 30 aa, Synthetic construct
PSIGNVARTLTRAEIEDELIRAGIESNPGP

SEQ ID NO: 62 - DNA, 90 bp, Synthetic construct
  source 1..90: mol_type = other DNA
ggacaaaggacgactgaacagatagttacggcccaggggtgggttccggatttgactgtggatggagatgttgagtcaaatcccggaccc

SEQ ID NO: 63 - protein, 30 aa, Synthetic construct
GQRTTEQIVTAQGWVPDLTVDGDVESNPGP

SEQ ID NO: 64 - DNA, 90 bp, Synthetic construct
  source 1..90: mol_type = other DNA
acgcgtggtggtttacgacggcaaaatattattggtggtgggcagaaggatttgacacaagatggtgacatcgagtcgaatcctgggccc

SEQ ID NO: 65 - protein, 30 aa, Synthetic construct
TRGGLRRQNIIGGGQKDLTQDGDIESNPGP

SEQ ID NO: 66 - DNA, 90 bp, Synthetic construct
  source 1..90: mol_type = other DNA
ggacaacggacaactgagcagatcgtgactgcacaaggttgggccccggatttgacacaggatggagatgtagagtcaaaccccggcccc

SEQ ID NO: 67 - protein, 30 aa, Synthetic construct
GQRTTEQIVTAQGWAPDLTQDGDVESNPGP

SEQ ID NO: 68 - DNA, 90 bp, Synthetic construct
  source 1..90: mol_type = other DNA
acacgtggtggtttacagcgtcaaaacattattggtggtggccaaagggatctgactcaagatggcgacatcgagtcgaaccccggccca

SEQ ID NO: 69 - protein, 30 aa, Synthetic construct
TRGGLQRQNIIGGGQRDLTQDGDIESNPGP

SEQ ID NO: 70 - DNA, 90 bp, Synthetic construct
  source 1..90: mol_type = other DNA
caaggcatcggtaagaagaatccgaaacaggaagctgcacgtcagatgttgctcttgttatcaggagatgttgagactaaccctggaccc

SEQ ID NO: 71 - protein, 30 aa, Synthetic construct
QGIGKKNPKQEAARQMLLLLSGDVETNPGP

SEQ ID NO: 72 - DNA, 90 bp, Synthetic construct
  source 1..90: mol_type = other DNA
actggttttttaaacaagttatatcattgtggctcatggactgacatattgttgttgttgtctggagatgtagaaaccaatccaggacct

SEQ ID NO: 73 - protein, 30 aa, Synthetic construct
TGFLNKLYHCGSWTDILLLLSGDVETNPGP

SEQ ID NO: 74 - DNA, 90 bp, Synthetic construct
  source 1..90: mol_type = other DNA
cgacgattgccggagtccgcccagctcccccaaggggcggggcgcggaagtctggtaacatgtggcgacgtggaggagaatccagggccc

SEQ ID NO: 75 - protein, 30 aa, Synthetic construct
RRLPESAQLPQGAGRGSLVTCGDVEENPGP

SEQ ID NO: 76 - DNA, 90 bp, Synthetic construct
  source 1..90: mol_type = other DNA
ttggagatgaaggagtctaatagtggttacgtagtcggtgaccgggggtctcttctcacttgtggggacgttgaatccaaccctggaccc

SEQ ID NO: 77 - protein, 30 aa, Synthetic construct
LEMKESNSGYVVGDRGSLLTCGDVESNPGP

SEQ ID NO: 78 - DNA, 90 bp, Synthetic construct
  source 1..90: mol_type = other DNA
acgcttatggggaacatcatgacacttgcagggtcaggtggtcggggaagcttgctgaccgcaggcgatgttgaaaagaaccctgggccc

SEQ ID NO: 79 - protein, 30 aa, Synthetic construct
TLMGNIMTLAGSGGRGSLLTAGDVEKNPGP

SEQ ID NO: 80 - DNA, 95 bp, Synthetic construct
  source 1..95: mol_type = other DNA
agaacagcgttcgatttccagcaggacgtttttcgctctaattatgacctactaaagttgtgcggtgatatcgagtctaatcctggacctgttac

SEQ ID NO: 81 - protein, 30 aa, Synthetic construct
RTAFDFQQDVFRSNYDLLKLCGDIESNPGP

SEQ ID NO: 82 - DNA, 90 bp, Synthetic construct
  source 1..90: mol_type = other DNA
atccatgctaatgattatcagatggctgtgtttaaatcaaattatgatttgctgaagttatgcggggacgtggaatcaaatcctggccct

SEQ ID NO: 83 - protein, 30 aa, Synthetic construct
IHANDYQMAVFKSNYDLLKLCGDVESNPGP

SEQ ID NO: 84 - DNA, 90 bp, Synthetic construct
  source 1..90: mol_type = other DNA
ttcttcgattcggtttgggtgtaccacttggcaaacagctcttgggttcgagatttaactagagaatgcattgaatctaaccctggacca

SEQ ID NO: 85 - protein, 30 aa, Synthetic construct
FFDSVWVYHLANSSWVRDLTRECIESNPGP

SEQ ID NO: 86 - protein, 8 aa, Synthetic construct
  VARIANT 2: note = X represents any amino acid
  VARIANT 4: note = X represents any amino acid
DXEXNPGP

SEQ ID NO: 87 - DNA, 280 bp, Synthetic construct
  source 1..280: mol_type = other DNA
agtcccctcaggatatagtagtttcgcttttgcatagggagggggaaatgtagccttatgcaatactcttgtagtcttgcaacatgcttatgtaacgatgagttagcaacatgccttacaaggagagaaaaagcaccgtgcatgccgattggtggaagtaaggtggtacgatcgtgccttattaggaaggcaacagacgggtctgacatggattggacgaaccaccgaattcgcattgcagagagtattgtatttaagtgcctagctcgatacaataaac

SEQ ID NO: 88 - DNA, 1503 bp, Synthetic construct
  source 1..1503: mol_type = other DNA
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 89 - protein, 500 aa, Synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQ

SEQ ID NO: 90 - DNA, 1503 bp, Synthetic construct
  source 1..1503: mol_type = other DNA
atgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 91 - protein, 500 aa, Synthetic construct
MGARASVLSGGELDKWEKIRLRPGGKKQYKLKHIVWASRELERFAVNPGLLETSEGCRQILGQLQPSLQTGSEELRSLYNTIAVLYCVHQRIDVKDTKEALDKIEEEQNKSKKKAQQAAADTGNNSQVSQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGSDPSSQ

SEQ ID NO: 92 - DNA, 3012 bp, Synthetic construct
  source 1..3012: mol_type = other DNA
ttttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaa

SEQ ID NO: 93 - protein, 1003 aa, Synthetic construct
FFREDLAFPQGKAREFSSEQTRANSPTRRELQVWGRDNNSLSEAGADRQGTVSFSFPQITLWQRPLVTIKIGGQLKEALLDTGADDTVLEEMNLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKQKKSVTVLDVGDAYFSVPLDKDFRKYTAFTIPSINNETPGIRYQYNVLPQGWKGSPAIFQCSMTKILEPFRKQNPDIVIYQYMDDLYVGSDLEIGQHRTKIEELRQHLLRWGFTTPDKKHQKEPPFLWMGYELHPDKWTVQPIVLPEKDSWTVNDIQKLVGKLNWASQIYAGIKVRQLCKLLRGTKALTEVVPLTEEAELELAENREILKEPVHGVYYDPSKDLIAEIQKQGQGQWTYQIYQEPFKNLKTGKYARMKGAHTNDVKQLTEAVQKIATESIVIWGKTPKFKLPIQKETWEAWWTEYWQATWIPEWEFVNTPPLVKLWYQLEKEPIIGAETFYVDGAANRETKLGKAGYVTDRGRQKVVPLTDTTNQKTELQAIHLALQDSGLEVNIVTDSQYALGIIQAQPDKSESELVSQIIEQLIKKEKVYLAWVPAHKGIGGNEQVDKLVSAGIRKVLFLDGIDKAQEEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIESMNKELKKIIGQVRDQAEHLKTAVQMAVFIHNFKRKGGIGGYSAGERIVDIIATDIQTKELQKQITKIQNFRVYYRDSRDPVWKGPAKLLWKGEGAVVIQDNSDIKVVPRRKAKIIRDYGKQMAGDDCVASRQDED

SEQ ID NO: 94 - DNA, 3012 bp, Synthetic construct; identical to SEQ ID NO: 92
  source 1..3012: mol_type = other DNA

SEQ ID NO: 95 - protein, 1003 aa, Synthetic construct; identical to SEQ ID NO: 93

SEQ ID NO: 96 - DNA, 261 bp, Synthetic construct
  source 1..261: mol_type = other DNA
atggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcaacccacctcccaatcccgaggggacccgacaggcccgaaggaatag

SEQ ID NO: 97 - protein, 86 aa, Synthetic construct
MEPVDPRLEPWKHPGSQPKTACTNCYCKKCCFHCQVCFMTKALGISYGRKKRRQRRRAHQNSQTHQASLSKQPTSQSRGDPTGPKE

SEQ ID NO: 98 - DNA, 351 bp, Synthetic construct
  source 1..351: mol_type = other DNA
atggcaggaagaagcggagacagcgacgaagagctcatcagaacagtcagactcatcaagcttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatag

SEQ ID NO: 99 - protein, 116 aa, Synthetic construct
MAGRSGDSDEELIRTVRLIKLLYQSNPPPNPEGTRQARRNRRRRWRERQRQIHSISERILSTYLGRSAEPVPLQLPPLERLTLDCNEDCGTSGTQGVGSPQILVESPTVLESGTKE

SEQ ID NO: 100 - DNA, 11233 bp, Synthetic construct
  source 1..11233: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaacctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaaggatgatcctgacgacggagaccgccgtcgtcgacaagccggatagtactgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccacagcctccttgggcaacctgttcagtgcgtcaccaccctctgggggaaaaactgcctcctcatatccaacccaaacctcccctgtctcagtgtaaagccattcccccttgtcctatcaagggggagtttgctgtgacattgttggtctggggtgacacatgtttgccaattcagtgcatcacggagaggcagatcttggggataaggaagtgcaggacagcatggacgtgggacatgcaggtgttgagggctctgggacactctccaagtcacagcgttcagaacagccttaaggataagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgagaatattgtttaaacccgcgtttaaccaaaatcatcccaaacttcccacccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggactagtctctctcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttgcttatgtaaaccagggcgcctataaaagagtgctgattttttgagtaaacttcaattccacaacacttttgtcttataccaactttccgtaccacttcctaccctcgtaaagaattcgcggccgcacgcgcagccgagccacatcgctcaacgcgtgccaccatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaaggcgcgcctagttggatcacactctttccctacacgacgctcttccgatcttctagattcgagagagaaatgttcctagtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccggcggccgccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgcccgctggatccggctccggcgagggcaggggaagtcttctaacatgcggggacgtggaggaaaatcccggcccaatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgtaagcccggtgcctgaggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtcccgggcgattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttc

SEQ ID NO: 101 - DNA, 11078 bp, Synthetic construct
  source 1..11078: mol_type = other DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaacctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctgatcaactggataactcaagcccggcttgtcgacgacggcggtctccgtcgtcaggatcatcccagccgagccacatcgctcaagccaccatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaaccgggtaagatccagacatgataagatacattgatgagtttggacaaaccacaactagaatgcagtgaaaaaaatgctttatttgtgaaatttgtgatgctattgctttatttgtaaccattataagctgcaataaacactagggacaggattggtgacagatagtactgagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccacagcctccttgggcaacctgttcagtgcgtcaccaccctctgggggaaaaactgcctcctcatatccaacccaaacctcccctgtctcagtgtaaagccattcccccttgtcctatcaagggggagtttgctgtgacattgttggtctggggtgacacatgtttgccaattcagtgcatcacggagaggcagatcttggggataaggaagtgcaggacagcatggacgtgggacatgcaggtgttgagggctctgggacactctccaagtcacagcgttcagaacagccttaaggataagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgagaatattgtttaaacccgtgttcacatgtctggatcgattggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggactagtctctctccgcgcctagttggatcacactctttccctacacgacgctcttccgatcttctagattcgagagagaaatgttcctagtggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccggcggccgccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgcccgctggatccggctccggcgagggcaggggaagtcttctaacatgcggggacgtggaggaaaatcccggcccaatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgtaagcccggtgcctgaggatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtcccgggcgattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttc

(19) United States
(12) Patent Application Publication
(10) Pub. No.: US 2026/0226501 A1
(43) Pub. Date: Aug. 6, 2026

(54) VIRAL PARTICLE PRODUCER CELLS WITH LANDING PAD-INTEGRATED VIRAL VECTORS

(71) Applicant: Fred Hutchinson Cancer Center, Seattle, WA (US)

(72) Inventors: Bernadeta Dadonaite, Seattle, WA (US); Caelan Radford, Seattle, WA (US); Jesse Bloom, Seattle, WA (US); Arjun Aditham, Seattle, WA (US); Arvind R. Subramaniam, Seattle (IN); Maria Toro Moreno, Seattle (ES)

(73) Assignee: Fred Hutchinson Cancer Center, Seattle, WA (US)

(21) Appl. No.: 19/149,494

(22) PCT Filed: Jan. 18, 2024
(86) PCT No.: PCT/US2024/012042

§ 371 Date: Jul. 18, 2025

Related U.S. Application Data
(60) Provisional application No. 63/480,459, filed on Jan. 18, 2023.

(51) Int. Cl. C12N 15/86

(52) U.S. Cl. CPC C12N 15/86; C12N 2750/14143; C12N 2750/14152

(57) ABSTRACT

Systems and methods to maintain a genotype-phenotype link during the production of viral particles are described. Cells are genetically modified to include a landing pad with an integrase attachment site within the cell's genome. The landing pad provides a site within the genome for a viral vector to reliably integrate. The landing pad allows for the integration of one viral vector per cell. Viral vectors include a barcode and a sequence encoding a protein of interest, with the barcode and protein of interest being associated through sequencing before integration into landing pads within cells.

### CROSS-REFERENCE TO RELATED APPLICATIONS

This application is a U.S. National Phase based on International Patent Application No. PCT/US2024/012042, filed on Jan. 18, 2024, which claims priority to U.S. Provisional Patent Application No. 63/480,459 filed Jan. 18, 2023, both of which are incorporated by reference herein in their entirety as if fully set forth herein.

### STATEMENT OF GOVERNMENT SUPPORT

This invention was made with government support under GM119835, and AI141707 awarded by the National Institutes of Health, and 1846521 awarded by the National Science Foundation. The government has certain rights in the invention.

### REFERENCE TO SEQUENCE LISTING

The Sequence Listing associated with this application is provided in XML format in lieu of a paper copy and is hereby incorporated by reference into the specification. The name of the file containing the Sequence Listing is 3GH4718.XML. The file is 140,827 bytes, was created on Jul. 14, 2025, and is being submitted electronically via Patent Center.

### FIELD OF THE DISCLOSURE

The current disclosure provides viral particle producer cells with landing pad-integrated viral vectors. The landing pad-integrated viral vectors include or encode a barcode, a protein or RNA of interest and include at least a portion of a viral genome. The viral particle producer cells with landing pad-integrated viral vectors can be utilized as genotype-phenotype linked libraries for a broad variety of purposes including in deep mutational scanning of proteins, decoding B-cell and T-cell receptor-ligand interactions, high-throughput screens for host factors affecting gene expression and viral replication.

### BACKGROUND OF THE DISCLOSURE

Viral vectors have been widely used for a variety of research and therapeutic purposes. Lentiviral vectors particularly have been widely used due to several beneficial features. For example, lentivirus can transduce both dividing and non-dividing cells, and can infect cells of different origins. Further, the RNA genome capacity of lentivirus allows it to deliver large gene sequences. These advantages of lentivirus make it widely applied in research and therapy.

Generally, to produce lentivirus for a research or therapeutic use, a transfer plasmid, which encodes a protein or RNA of interest, and other helper plasmids, which include viral genes required for lentivirus packaging, are co-transduced into cells. Many approaches utilize four separate plasmids. The first includes the transfer plasmid encoding the protein or RNA of interest. The remainder are helper plasmids. The first helper plasmid encodes a lentiviral group specific antigen (GAG) gene and a lentiviral polymerase (POL) protein; the second helper plasmid encodes an envelope protein (usually Vesicular Stomatitis Virus Glycoprotein (VSV-G)); and the third helper plasmid encodes an HIV regulator of expression of virion protein (Rev) protein. After transfection with these plasmids, lentivirus particles including the gene encoding the protein or RNA of interest will be released into a culture medium, and the culture medium can then be harvested for the lentiviral particles.

In many research protocols the gene encoding the protein or RNA of interest is linked to a nucleic acid “barcode” and sequencing the barcode should tell a researcher the linked protein of interest. This linkage can be referred to herein as the “genotype-phenotype” link. However, the pseudodiploid nature of the lentiviral genome leads to frequent genome recombination, which erodes genotype-phenotype links between barcodes and proteins of interest. This loss of genotype-phenotype link hinders research efforts based on produced viral particles.

### SUMMARY OF THE DISCLOSURE

The current disclosure provides systems and methods to maintain a genotype-phenotype link during the production of viral particles. The disclosed systems and methods utilize several steps and approaches to maintain this link.

As one step to assist in maintaining a genotype-phenotype link, transfer plasmids including a barcode and a sequence encoding a protein or RNA of interest are generated and the genotype-phenotype link is sequenced and recorded before the transfer plasmid is transfected into a cell.

As another step in assisting in maintaining a genotype-phenotype link, cells are genetically modified to include a landing pad at a selected locus within the cell's genome. The landing pad includes at least an integrase attachment site. The landing pad can additionally include a promoter, a selection marker, insulator sequences, and a portion of the viral genome (e.g., the 5′ UTR and packaging signals). The landing pad provides a site within the genome for a transfer plasmid to reliably integrate. The landing pad also only allows for the integration of one transfer plasmid in each cell.

As another step in assisting in maintaining a genotype-phenotype link, transfer plasmids (also referred to herein as viral vectors) are designed to integrate into the landing pad. The viral vectors include an integrase attachment site to direct the viral vector to the landing pad for integration into the genome, a barcode, a sequence encoding a protein or RNA of interest, and all or a portion of the lentiviral genome (the amount of viral genome selected to be compatible with the portion of the lentiviral genome already present within a landing pad). Sequences encoding a protein or RNA of interest can be located either within or outside the viral genome.

The systems and methods are practiced such that only one viral vector will integrate into a landing pad, such that all viral particles produced by the cell following transfection with helper plasmids will contain the same genotype-phenotype link.

These advances increase the experimental power of viral libraries used for a variety of purposes, such as deep mutational scanning of proteins, decoding B-cell and T-cell receptor-ligand interactions, high-throughput screens for host factors affecting gene expression and viral replication.

### BRIEF DESCRIPTION OF THE SEVERAL VIEWS OF THE DRAWINGS

Some of the drawings submitted herein may be better understood in color. Applicant considers the color versions of the drawings as part of the original submission and reserves the right to present color images of the drawings in later proceedings.

FIGS. 1A, 1B. (1A) Previous approach to preparing viral entry protein deep mutational scanning libraries. The depicted approach requires a 1-to-1 linkage between viral glycoprotein mutant and barcode. Transfections introduce multiple plasmids into single cells and can vary from experiment. There is a requirement of vesicular stomatitis virus glycoprotein (VSV-G) pseudotype lentivirus to generate an ‘initial virus’ population. This step mixes glycoprotein mutants and barcodes and requires a low-MOI (multiplicity of infection) step. During the low MOI step, template switching can occur, which scrambles barcodes and viral entry protein (VEP) mutants, introducing additional mutations into the library. The VEP sequence is also required to be in the lentiviral genome, which can impinge titers and limit the VEPs that can be studied in this system. Technical artifacts and errors can occur during steps to sequence virus library to link VEP mutant and barcode, and during the low MOI step, integration of viral genomes is pseudorandom throughout the cell genome, possibly creating loss of library members or inadequate representation. (1B) Advantages of the disclosed landing pad approach: the landing pad approach allows storage of libraries of viruses for packaging from transfection alone. Each cell will recombine only *one* copy of a viral vector. The landing pad approach circumvents the initial low MOI step. This circumvention (1) prevents template switching that can scramble mutants and barcodes and (2) allows placement of proteins of interest outside of the lentiviral genome. Further, the smaller lentiviral genome should allow higher titers due to more efficient packaging, and enable study of proteins of interest that are large. Because integration occurs at a specific “safe harbor” locus, all mutants should be consistently expressed. The “safe harbor” locus can be chosen to allow for strong expression reducing or preventing any dropout of library members.

FIGS. 2A-2C. Production of lentiviruses from landing pads. (2A) The lentivirus genome-containing vector is transfected into landing pad-containing cells to make a cell line where each cell expresses a single copy of the lentiviral genome. (2B) Protocol for generating lentiviruses from landing pad cells. (2C) Successful generation of VSV-G pseudotyped lentiviruses from landing pads.

FIGS. 3A-3C. Expression of mCherry using a landing pad promoter. (3A) Vector design; (3B) mCherry (induced); (3C) zsGreen (constitutive).

FIG. 4. Integrated lentiviral vectors within landing pads can be used to generate pseudotyped lentiviral particles.

FIG. 5. Titers suggest virus production can be limited by lentiviral genomes.

FIGS. 6A, 6B. (6A) Schematic of landing pad plasmid. (6B) Different versions of the lentiviral genome provide higher titers with sample VEP (VSV-G) in trans.

FIG. 7. (top) Flow cytometry of HEK293T cells with a landing pad (“v5”) 1 week after transfection. Lentiviral vector encoding attB sites is successfully integrated. (bottom) Target cells transduced with viruses packaged from integrated genomes (v5::) or through conventional packaging (v5−).

FIG. 8. Titers of lentiviral vectors with different promoter combinations. The positive control pos_ctrl is lentiviral vector (pLJM1-eGFP) conventionally packaged.

FIG. 9. Sequences supporting the disclosure include cHS4 insulator (SEQ ID NO: 1), CMV promoter (SEQ ID NO: 2), CMV-immediate early (IE) promoter (SEQ ID NO: 3), ZsGreen Fluorescent Protein Encoding Sequence (SEQ ID NO: 4), Bxb1 recombinase (SEQ ID NO: 5), Bxb1-attB recombination site-version 1 (SEQ ID NO: 6), Bxb1-attB recombination site-version 2 (SEQ ID NO: 7), attB_Forward (SEQ ID NO: 9), attB (reversed) (a reverse complement of the attB_Forward for recombining plasmids in the opposing direction) (SEQ ID NO: 10), Bxb1-attP recombination site-version 1 (SEQ ID NO: 11), Bxb1-attP recombination site-version 2 (SEQ ID NO: 12), Bxb1-attP recombination site-version 3 (SEQ ID NO: 13), Flp (Flippase) (SEQ ID NO: 14), Variant of Flp (Flippase)-version 1 (SEQ ID NO: 15), Variant of Flp (Flippase)-version 2 ((SEQ ID NO: 16), FRT recognition site (SEQ ID NO: 17), Cre (SEQ ID NO: 18), LoxP site (SEQ ID NO: 19), Lox2272 (SEQ ID NO: 20), Lox511 (SEQ ID NO: 21), Lox 66 (SEQ ID NO: 22), Lox71 (SEQ ID NO: 23), LoxM2 (SEQ ID NO: 24), Lox5171 (SEQ ID NO: 25), VCre recombinase (SEQ ID NO: 26), VloxP recognition site (SEQ ID NO: 27), Dre recombinase (SEQ ID NO: 28), rox recognition site (SEQ ID NO: 29), piggyBac (PB) transposase (GenBank ABS12111.1) [Trichoplusia ni]: (SEQ ID NO: 30), Frog Prince transposase (GenBank: AAP49009.1) [Rana pipiens]: (SEQ ID NO: 31), TcBuster transposase GenBank: ABF20545.1) [Tribolium castaneum]: (SEQ ID NO: 32), Tol2 transposase (GenBank: BAA87039.1) [Oryzias latipes]: (SEQ ID NO: 33), Sleeping Beauty transposase enzyme (SEQ ID NO: 34), Hyperactive Sleeping Beauty is SB100X (SEQ ID NO: 35), Sleeping beauty IR/DR sequence, integration junction (chr7, 79796094) (SEQ ID NO: 36), Sleeping beauty IR/DR sequence, Integration junction (repeat region) (SEQ ID NO: 37), IR/DR encoding sequence Sleeping Beauty (SEQ ID NO: 38), IR/DR and chromosomal sequence Sleeping Beauty (SEQ ID NO: 39), IR/DR and chromosomal sequence Sleeping Beauty (SEQ ID NO: 40), IR/DR of Sleeping Beauty (SEQ ID NO: 41), IR/DR and chromosomal sequence of Sleeping Beauty (SEQ ID NO: 42), IR/DR of Sleeping Beauty (SEQ ID NO: 43), exemplary U3 sequence; Rous sarcoma virus U3 (SEQ ID NO: 87), Examples of Gag, Pol, Tat, and Rev sequences; HIV-1 gag nucleotide sequence from plasmid psPAX2 (SEQ ID NO: 88), Examples of Gag, Pol, Tat, and Rev sequences; HIV-1 gag protein sequence from plasmid psPAX2 (SEQ ID NO: 89), Examples of Gag, Pol, Tat, and Rev sequences; HIV-1 gag nucleotide sequence from plasmid pNL4-3 (GenBank accession no. AF324493.2) (SEQ ID NO: 90) Examples of Gag, Pol, Tat, and Rev sequences; HIV-1 gag protein sequence from plasmid pNL4-3 (GenBank accession no. AAK08483.1) (SEQ ID NO: 91), Examples of Gag, Pol, Tat, and Rev sequences; HIV-1 pol nucleotide sequence from plasmid psPAX2 (SEQ ID NO: 92), Examples of Gag, Pol, Tat, and Rev sequences; HIV-1 pol protein sequence from psPAX2 (SEQ ID NO: 93), Examples of Gag, Pol, Tat, and Rev sequences; HIV-1 pol nucleotide sequence from plasmid pNL4-3 (GenBank accession no. AF324493.2) (SEQ ID NO: 94), Examples of Gag, Pol, Tat, and Rev sequences; HIV-1 pol protein sequence from plasmid pNL4-3 (GenBank accession no. AAK08484.2) (SEQ ID NO: 95), Examples of Gag, Pol, Tat, and Rev sequences; HIV-1 Tat coding sequence from plasmid pNL4-3 (GenBank accession no. AF324493.2) (SEQ ID NO: 96), Examples of Gag, Pol, Tat, and Rev sequences; HIV-1 Tat protein sequence from plasmid pNL4-3 (GenBank accession no. AAK08486.1) (SEQ ID NO: 97), Examples of Gag, Pol, Tat, and Rev sequences; HIV-1 Rev coding sequence from plasmid pNL4-3 (GenBank accession no. AF324493.2) (SEQ ID NO: 98), Examples of Gag, Pol, Tat, and Rev sequences; HIV-1 Rev protein sequence from plasmid pNL4-3 (GenBank accession no. AAK08487.1) (SEQ ID NO: 99), Example Viral Vector Sequence 1 (SEQ ID NO: 100), and Example Viral Vector Sequence 2 (SEQ ID NO: 101).

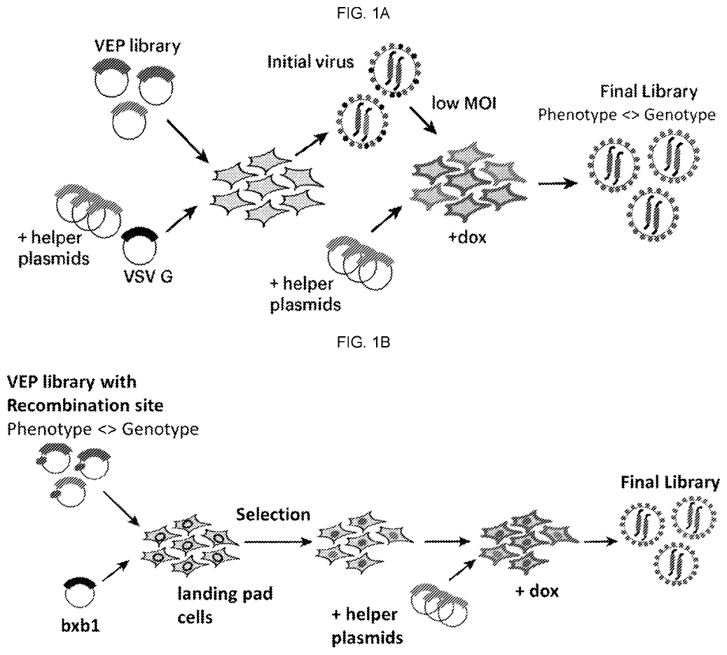

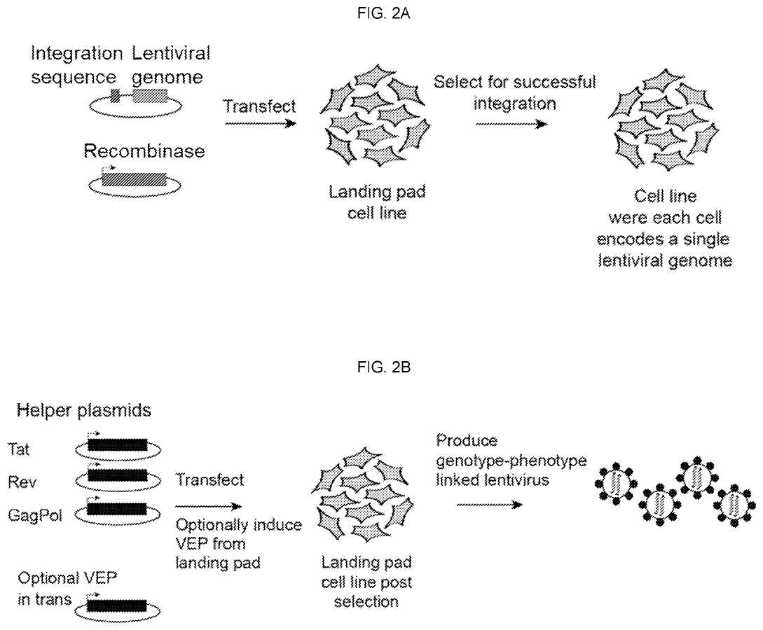

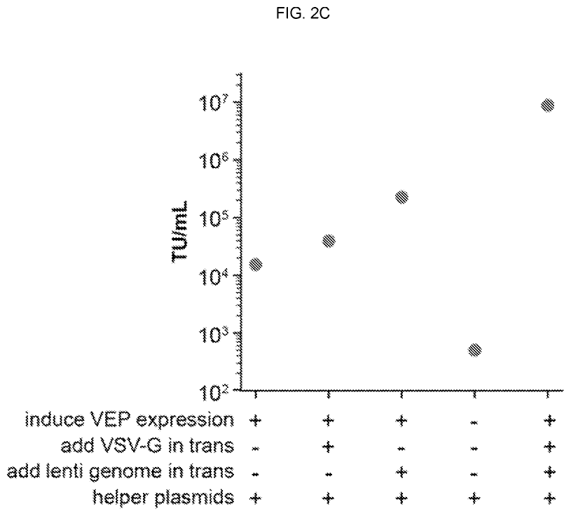

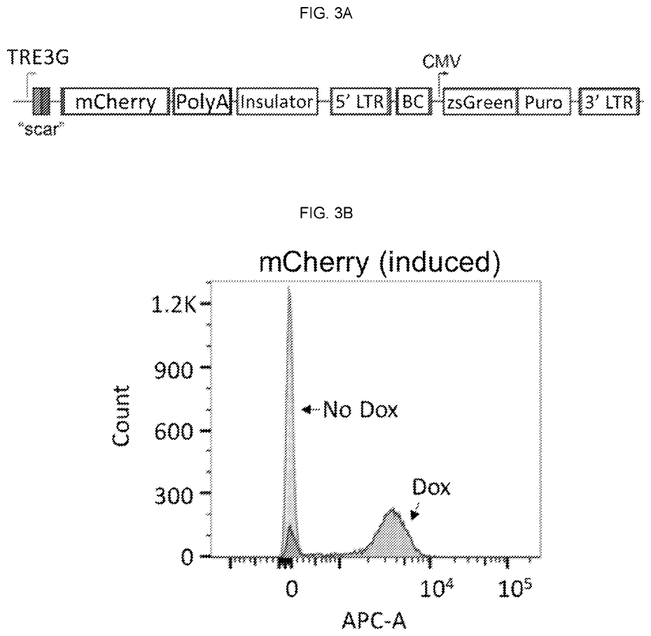

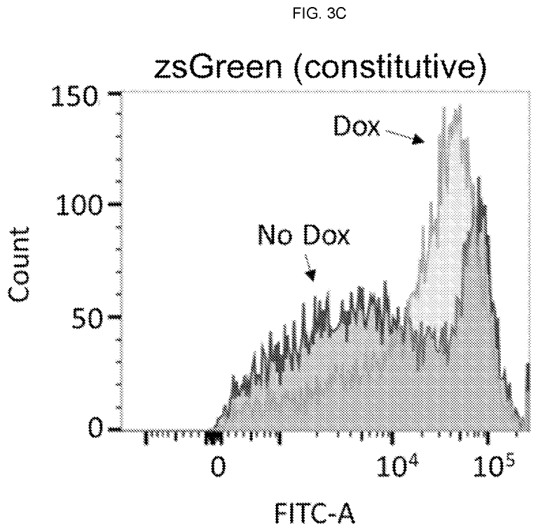

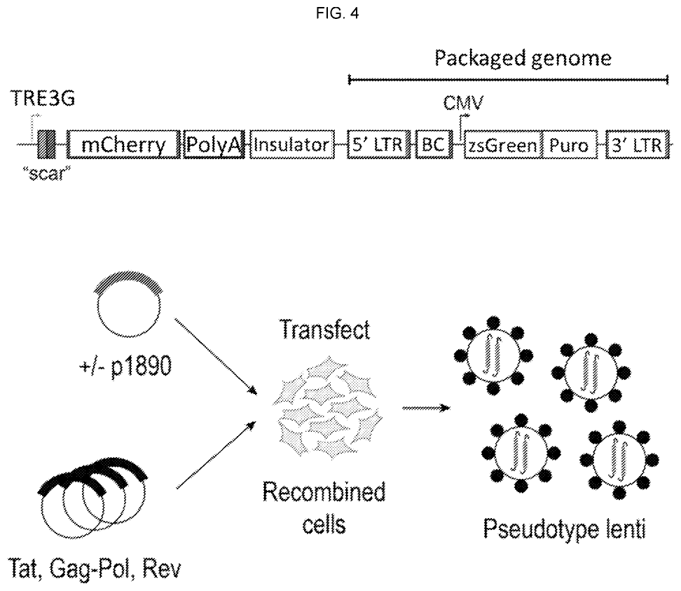

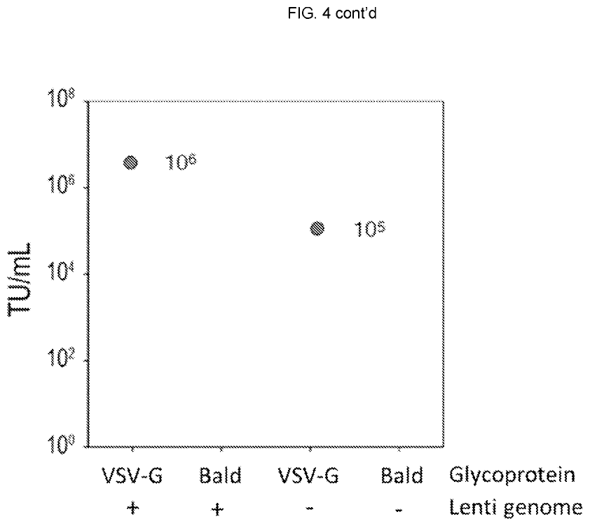

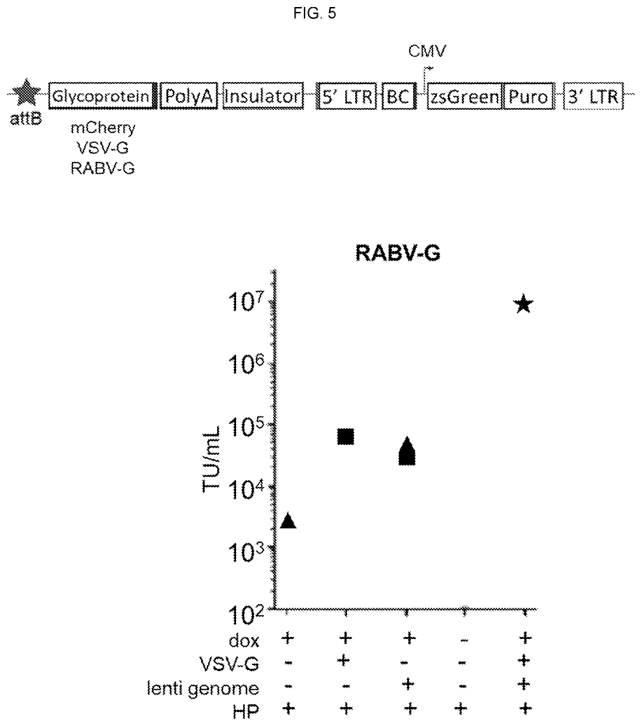

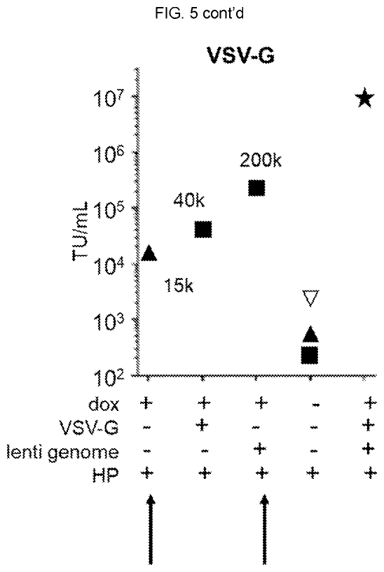

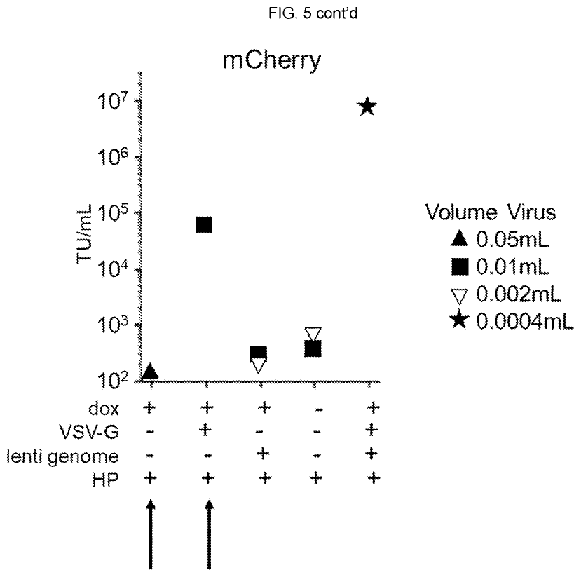

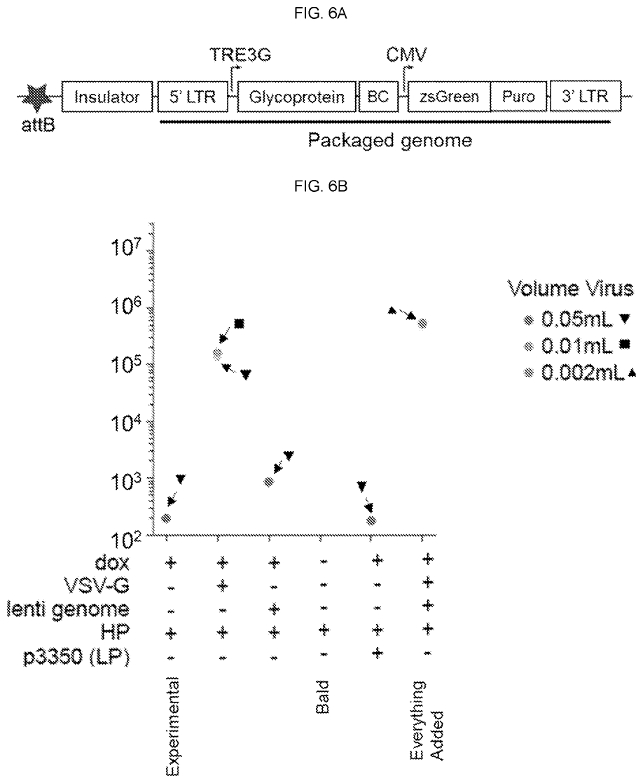

### DETAILED DESCRIPTION

Viral vectors have been widely used for a variety of research and therapeutic purposes. Lentiviral vectors particularly have been widely used due to several beneficial features. For example, lentivirus can transduce both dividing and non-dividing cells, and can infect cells of different origins. Further, the RNA genome capacity of lentivirus is 10 Kb, which is more flexible for delivering larger gene sequences. These advantages of lentivirus make it widely applied in research and therapy.

Generally, to produce lentivirus for a research or therapeutic use, a transfer plasmid, which encodes a protein of interest, and other helper plasmids, which include viral genes required for lentivirus packaging, are transiently transfected into cells. Many approaches utilize co-transfection of four separate plasmids. The first includes the transfer plasmid encoding the protein of interest. The remainder are helper plasmids. The first helper plasmid encodes a lentiviral group specific antigen (GAG) gene and a lentiviral polymerase (POL) protein; the second helper plasmid encodes an envelope protein (usually Vesicular Stomatitis Virus Glycoprotein (VSV-G)); and the third helper plasmid encodes an HIV regulator of expression of virion protein (Rev) protein. After transfection with these plasmids, lentivirus particles including the gene encoding the protein of interest will be released into a culture medium, and the culture medium can then be harvested for the lentiviral particles.

In many research protocols the gene encoding the protein or RNA of interest is linked to a nucleic acid “barcode” and sequencing the barcode should tell a researcher the linked protein of interest. This linkage can be referred to herein as the “genotype-phenotype” link. However, the pseudodiploid nature of the lentiviral genome leads to frequent genome recombination, which erodes genotype-phenotype links between barcodes and proteins of interest. This loss of genotype-phenotype link hinders research efforts based on produced viral particles.

The current disclosure provides systems and methods to maintain a genotype-phenotype link during the production of lentiviral particles. The disclosed systems and methods utilize several steps and approaches to maintain this link.

As one step to assist in maintaining a genotype-phenotype link, transfer plasmids including a barcode and a sequence encoding a protein of interest are generated and the genotype-phenotype link is sequenced and recorded before the transfer plasmid is transfected into a cell.

As another step in assisting in maintaining a genotype-phenotype link, cells are genetically modified to include a landing pad at a selected locus within the cell's genome. The landing pad includes at least an integrase attachment site. The landing pad can additionally include a promoter, a selection marker, insulator sequences, and a portion of the lentiviral genome (e.g., the 5′ UTR and packaging signals). The landing pad provides a site within the genome for a transfer plasmid to reliably integrate. The landing pad also only allows for the integration of one transfer plasmid in each cell.

As another step in assisting in maintaining a genotype-phenotype link, transfer plasmids (also referred to herein as viral vectors) are designed to integrate into the landing pad. The viral vectors include an integrase attachment site to direct the viral vector to the landing pad for integration into the genome, a barcode, a sequence encoding a protein of interest, and all or a portion of the lentiviral genome (the amount of viral genome selected to be compatible with the portion of the lentiviral genome already present within a landing pad).

The systems and methods are practiced such that only one viral vector will integrate into a landing pad, such that all viral particles produced by the cell following transfection with helper plasmids will contain the same genotype-phenotype link.

These advances increase the experimental power of lentiviral libraries used for a variety of purposes, such as deep mutational scanning of proteins, decoding B-cell and T-cell receptor-ligand interactions, identifying factors affecting viral gene expression, and high-throughput gene expression screens.

Aspects of the disclosure are now described with additional detail and options as follows: (i) Mammalian Cells Lines; (ii) Landing Pads; (iii) Viral Vectors for Integration into Landing Pads; (iv) Targeted Genetic Engineering; (v) Packaging Vectors; (vi) Uses of Genotype-Phenotype Linked Libraries; (vii) Kits; (viii) Exemplary Embodiments; (ix) Experimental Examples; and (x) Closing Paragraphs. These headings are provided for organizational purposes only and do not limit the scope or interpretation of the disclosure.

(i) Mammalian Cell Lines. Particular embodiments disclosed herein utilize mammalian cells as viral particle producer cells. these cells include a landing pad-integrated viral vector.

Different mammalian cell lines can be used as viral particle producer cells. The term “mammalian cell” includes cells from any member of the order Mammalia, such as, for example, human cells, mouse cells, rat cells, monkey cells, hamster cells, and the like.

Particular examples of mammalian cells and cell lines that can be used within embodiments disclosed herein include HEK293T (293T) and related cell lines (e.g., HEK293T/17 aka HEK293T clone 17, HEK293F, HEK293S, HEK293SGH, EK293FTM, HEK293SGGD, GP2-293, HEK293T Lenti-X); HeLa and related cell lines (e.g., HeLa S3, HeLa B, HeLa T4); Chinese Hamster Ovary (COS) and related cell lines (e.g., COS-1, COS-6, COS-M6A, COS-7); A549; MDCK; HepG2; C2C12; THP-1; HUDEP-2; C8161; CCRF-CEM; MOLT; mIMCD-3; NHDF; Huh1; Huh4; Huh7; HUVEC; HASMC; HEKn; HEKa; MiaPaCell; Panc1; PC-3; TF1; CTLL-2; C1R; Rat6; CV1; RPTE; A10; T24; J82; A375; ARH-77; Calu1; SW480; SW620; SKOV3; SK-UT; CaCo2; P388D1; SEM-K2; WEHI-231; HB56; TIB55; Jurkat; J45.01; LRMB; Bcl-1; BC-3; IC21; DLD2; Raw264.7; NRK; NRK-52E; MRC5; MEF; BS-C-1; monkey kidney epithelial; BALB/3T3 mouse embryo fibroblast; 3T3 Swiss; 3T3-L1; 132-d5 human fetal fibroblasts; 10.1 mouse fibroblasts; 3T3; 721; 9L; A2780; A2780ADR; A2780cis; A172; A20; A253; A431; A-549; ALC; B16; B35; BCP-1; BEAS-2B; bEnd.3; BHK-21; BR 293; BxPC3; C3H-10T1/2; C6/36; Cal-27; CHO; CHO-7; CHO-IR; CHO-K1; CHO-K2; CHO-T; CHO Dhfr−/−; COR-L23; COR-L23/CPR; COR-L23/5010; COR-L23/R23; COV-434; CML T1; CMT; CT26; D17; DH82; DU145; DuCaP; EL4; EM2; EM3; EMT6/AR1; EMT6/AR10.0; FM3; H1299; H69; HB54; HB55; HCA2; Hepa1c1c7; HL-60; HMEC; HT-29; JY; K562; Ku812; KCL22; KG1; KYO1; LNCap; Ma-Mel 1-48; MC-38; MCF-7; MCF-10A; MDA-MB-231; MDA-MB-468; MDA-MB-435; MDCK II; MOR/0.2R; MONO-MAC 6; MTD-1A; MyEnd; NCI-H69/CPR; NCI-H69/LX10; NCI-H69/LX20; NCI-H69/LX4; NIH-3T3; NALM-1; NW-145; OPCN/OPCT cell lines; Peer; PNT-1A/PNT2; RenCa; RIN-5F; RMA/RMAS; Saos-2; Sf-9; SkBr3; T2; T-47D; T84; THP1; U373; U87; U937; VCaP; Vero; WM39; WT-49; X63; YAC-1; and YAR.

In particular embodiments, the cell is a mouse cell, a human cell, a Chinese hamster ovary (CHO) cell, a CHOK1 cell, a CHO-DXB11 cell, a CHO-DG44 cell, a CHOK1SV cell including all variants (e.g. POTELLIGENT®, Lonza, Slough, UK), or a CHOK1SV GS-KO (glutamine synthetase knockout) cell including all variants (e.g., XCEED™ Lonza, Slough, UK). Exemplary human cells include human embryonic kidney (HEK) cells (such as HEK293), a HeLa cell, or a HT1080 cell.

In particular embodiments, cells within cell lines suitable for use with the systems and methods disclosed herein are transfected with a landing pad, a viral vector, and/or helper plasmids. “Transfection” refers to the introduction of an exogenous nucleic acid molecule, such as a landing pad, into a cell. A “transfected” cell includes an exogenous nucleic acid molecule inside the cell. The transfected nucleic acid molecule can be integrated into the host cell's genomic DNA. Host cells that express exogenous nucleic acid molecules or fragments thereof are referred to as “recombinant,” “transformed,” or “transgenic”. A number of transfection techniques are generally known in the art. See, e.g., Graham et al, Virology, 52:456 (1973); Sambrook et al, Molecular Cloning, a laboratory manual, Cold Spring Harbor Laboratories, New York (1989); Davis et al, Basic Methods in Molecular Biology, Elsevier (1986); and Chu et al, Gene 73:197 (1981). Suitably, transfection of a mammalian cell with one or more of the constructs described herein can utilize a transfection agent, such as polyethylenimine (PEI) or other suitable agent, including various lipids and polymers, to integrate the nucleic acids into the host cell's genomic DNA.

Mammalian cells can be within mammalian cell cultures which can be either adherent cultures or suspension cultures. Adherent cultures refer to cells that are grown on a substrate surface, for example a plastic plate, dish or other suitable cell culture growth platform, and may be anchorage dependent. Suspension cultures refer to cells that can be maintained in, for example, culture flasks or large suspension vats, which allows for a large surface area for gas and nutrient exchange. Suspension cell cultures often utilize a stirring or agitation mechanism to provide appropriate mixing. Media and conditions for maintaining cells in suspension are generally known in the art. An exemplary suspension cell culture includes human HEK293 clonal cells.

Methods of culturing mammalian cells are known in the art and include the use of various cell culture media, appropriate gas concentration/exchange and temperature control to promote growth of the cells and integration of the landing pads and viral vectors into the genome of the cell.

In particular embodiments, cells are maintained within a bioreactor unit that can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO2 levels, maintenance of pH level, agitation (e.g., stirring), and/or cleaning/sterilizing. Example bioreactor units can contain multiple bioreactors within the unit, for example the unit can have 1, 2, 3, 4, 5, 10, 15, 20, 25, 30, 35, 40, 45, 50, 60, 70, 80, 90, or 100, or more bioreactors in each unit and/or a facility may contain multiple units having a single or multiple bioreactors within the facility. In various embodiments, the bioreactor can be suitable for batch, semi fed-batch, fed-batch, perfusion, and/or a continuous fermentation process. Any suitable bioreactor diameter can be used. In embodiments, the bioreactor can have a volume between 100 mL and 50,000 L. Examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. Additionally, suitable bioreactors can be multi-use, single-use, disposable, or non-disposable and can be formed of any suitable material including metal alloys such as stainless steel (e.g., 316L or any other suitable stainless steel) and Inconel, plastics, and/or glass.

Methods of isolating the desired cells include various filtration techniques, including the use of sieves, filter apparatus, cell-selection apparatus and sorting, cell counting, etc.

(ii) Landing Pads. Particular embodiments include systems and methods for inserting DNA into a chromosomal locus in mammalian cells. First, a landing pad can be introduced into a selected locus. In particular embodiments, a selected locus is within a genomic safe harbor (GSH). The concept of GSH for genetic modification was first introduced in 2011 by Papapetrou and colleagues (Nature Biotechnology. 2011, 29 (1): 73-8). The major criteria proposed to define a GSH site are (1) the ability to accommodate new genetic material with, (2) predictable function, and (3) without potentially harmful alterations in host cell genomic activity. In particular embodiments, the GSH includes the Adeno-associated virus site 1 (AAVS1) locus.

The landing pad includes integrase attachment sites for recombination with sequences in the viral vector, thus directing the integration or “landing” of the viral vector at the selected genomic locus.

Many bacteriophage and integrative plasmids encode site-specific recombination systems that enable the stable incorporation of their genome into those of their hosts. In these systems, the minimal requirements for the recombination reaction are a recombinase enzyme, or integrase, which catalyzes the recombination event, and two recombination sites (Sadowski (1986) J. Bacteriol. 165:341-347; Sadowski (1993) FASEB J. 7:760-767). For phage integration systems, these are referred to as attachment (att) sites, with an attP element from phage DNA and the attB element encoded by the bacterial genome. The two attachment sites can share as little sequence identity as a few base pairs. The recombinase protein binds to both att sites and catalyzes a conservative and reciprocal exchange of DNA strands that result in integration a transfected construct into the host cell's DNA.

Recombinases can be classified into two distinct families: serine recombinases and tyrosine recombinases, based on distinct biochemical properties. Serine recombinases and tyrosine recombinases are further divided into bidirectional recombinases and unidirectional recombinases. Examples of bidirectional serine recombinases include β-six, CinH, ParA and γδ; and examples of unidirectional serine recombinases include Bxb1, φC31, TP901, TG1, φBT1, R4, φRV1, φFC1, MR11, A118, U153 and gp29. Examples of bidirectional tyrosine recombinases include Cre, FLP, and R; and unidirectional tyrosine recombinases include Lambda, HK101, HK022 and pSAM2. The terms “recombinase” and “integrase” are used interchangeably herein.

Recombinases can also be classified as irreversible or reversible. As used herein, an “irreversible recombinase” refers to a recombinase that can catalyze recombination between two complementary recombination sites, but cannot catalyze recombination between the hybrid sites that are formed by this recombination without the assistance of an additional factor. Thus, an “irreversible recognition site” refers to a recombinase recognition site that can serve as the first of two DNA recognition sequences for an irreversible recombinase and that is modified to a hybrid recognition site following recombination at that site. A “complementary irreversible recognition site” refers to a recombinase recognition site that can serve as the second of two DNA recognition sequences for an irreversible recombinase and that is modified to a hybrid recombination site following homologous recombination at that site. For example, attB and attP, described below, are the irreversible recombination sites for Bxb1 and phiC31 recombinases—attB is the complementary irreversible recombination site of attP, and vice versa.

In particular embodiments, the recombinase system includes the Bxb1 recombinase system. The mycobacteriophage large serine recombinase Bxb1 catalyzes site-specific recombination between its corresponding attP and attB recognition sites. In particular embodiments, the Bxb1 recombinase includes the sequence as set forth in SEQ ID NO: 5. In particular embodiments, the Bxb1 recombinase system includes the Bxb1-attB site sequence as set forth in SEQ ID NOs: 6, 7, 9, or 10 and the Bxb1-attP site sequence as set forth in SEQ ID NO: 11, 12, or 13. In particular embodiments, attB_Forward includes the sequence as set forth in SEQ ID NO: 9. In particular embodiments, attB (reverse) is a reverse complement of the attB_Forward sequence and includes the sequence as set forth in SEQ ID NO: 10. In particular embodiments, the viral vector includes an integrase binding site including a Bxb1 attB recombination site and the landing pad includes an integrase binding site including a Bxb1 attP recombination site. In particular embodiments, the viral vector includes an integrase binding site including a Bxb1 attP recombination site and the landing pad includes an integrase binding site including a Bxb1 attB recombination site.

Additional examples of recombinase systems include the Flp/Frt system, the Cre/loxP system, the Dre/rox system, the Vika/vox system, the PhiC31 system, Pa01, Pa03, Ec03, Ec04, Kp03, Ec06, Ec07, Sa02, Ef02, Kp05, Ef01, Kp04, Ec05, Sa01, Kp01, Sa03, Pa02, Sp56, Enc3, Pf80, Cp36, Dn29, Pc01, Enc9, and additional recombinase systems identified in Durrant et al., Nat Biotechnol, 2022, PMID: 36217031; and Yarnall et al., Nat Biotechnol, 2022, PMID: 36424489.

The Flp/Frt DNA recombinase system was isolated from Saccharomyces cerevisiae. The Flp/Frt system includes the recombinase Flp (flippase) that catalyzes DNA-recombination on its Frt recognition sites. In particular embodiments, Flp (flippase) includes the sequence SEQ ID NO: 14 and the FRT recognition site includes SEQ ID NO: 17.

Variants of the Flp protein include SEQ ID NO: 15 (GenBank: ABD57356.1) and SEQ ID NO: 16 (GenBank: ANW61888.1).

The Cre/loxP system is described in, for example, EP 02200009B1. Cre is a site-specific DNA recombinase isolated from bacteriophage P1. In particular embodiments, Cre includes the sequence SEQ ID NO: 18.

The recognition site of the Cre protein is a nucleotide sequence of 34 base pairs, the loxP site (SEQ ID NO: 19). Cre recombines the 34 bp loxP DNA sequence by binding to the 13 base pair inverted repeats and catalyzing strand cleavage and re-ligation within the spacer region. The staggered DNA cuts made by Cre in the spacer region are separated by 6 base pairs to give an overlap region that acts as a homology sensor to ensure that only recombination sites having the same overlap region recombine. Variants of the lox recognition site that can also be used include: lox2272 (SEQ ID NO: 20); lox511 (SEQ ID NO: 21); lox66 (SEQ ID NO: 22); lox71 (SEQ ID NO: 23); loxM2 (SEQ ID NO: 24); and lox5171 (SEQ ID NO: 25).

The VCre/VloxP recombinase system was isolated from Vibrio plasmid p0908. In particular embodiments, the VCre recombinase of this system includes SEQ ID NO: 26 and the VloxP recognition site includes SEQ ID NO: 27.

The sCre/SloxP system is described in WO 2010/143606. The Dre/rox system is described in U.S. Pat. Nos. 7,422,889 and 7,915,037B2. It generally includes a Dre recombinase isolated from Enterobacteria phage D6 with the sequence SEQ ID NO: 28 and the rox recognition site (SEQ ID NO: 29).

The Vika/vox system is described in U.S. Pat. No. 10,253,332. Additionally, the PhiC31 recombinase recognizes the AttB/AttP binding sites.

A number of transposases have been described in the art that facilitate insertion of nucleic acids into the genome of vertebrates, including humans. Examples of such transposases include sleeping beauty (“SB”, e.g., derived from the genome of salmonid fish); piggyback (e.g., derived from lepidopteran cells and/or the Myotis lucifugus); mariner (e.g., derived from Drosophila); frog prince (e.g., derived from Rana pipiens); Tol1; Tol2 (e.g., derived from medaka fish); TcBuster (e.g., derived from the red flour beetle Tribolium castaneum), Helraiser, Himar1, Passport, Minos, Ac/Ds, PIF, Harbinger, Harbinger3-DR, HSmar1, and spinON.

The PiggyBac (PB) transposase is a compact functional transposase protein. PIGGYBAC® is a 2475 bp short-inverted repeat element that has an asymmetric terminal repeat structure with a 3-bp spacer between the 5′ 13-bp TR (terminal repeat) and the 19-bp IR (internal repeat), and a 31-bp spacer between the 3′ TR and IR. The single 2.1 kb open reading frame encodes a functional transposase. PIGGYBAC® transposes via a unique cut-and-paste mechanism, inserting exclusively at 5′ TTAA 3′ target sites that are duplicated upon insertion, and excising precisely, leaving no footprint. For additional information regarding PB, see Fraser et al., Insect Mol. Biol., 1996, 5, 141-51; Mitra et al., EMBO J., 2008, 27, 1097-1109; Ding et al., Cell, 2005, 122, 473-83; and U.S. Pat. Nos. 6,218,185; 6,551,825; 6,962,810; 7,105,343; and 7,932,088. Hyperactive piggyBac transposases are described in U.S. Pat. No. 10,131,885.

In particular embodiments, PB transposase has the sequence as set forth in SEQ ID NO: 30 (GenBank ABS12111.1).

In particular embodiments, a Frog Prince transposase has the sequence as set forth in SEQ ID NO: 31 (GenBank: AAP49009.1). See also US2005/0241007.

In particular embodiments, a TcBuster transposase has the sequence as set forth in SEQ ID NO: 32 (GenBank: ABF20545.1).

In particular embodiments, a Tol2 transposase has the sequence set forth in SEQ ID NO: 33 (GenBank: BAA87039.1).

Additional information on DNA transposons can be found, for instance, in Munõz-López & Garcia Pérez, Curr Genomics, 11(2):115-128, 2010.

Sleeping Beauty is described in Ivics et al. Cell 91, 501-510, 1997; Izsvak et al., J. Mol. Biol., 302(1):93-102, 2000; Geurts et al., Molecular Therapy, 8(1): 108-117, 2003; Mates et al. Nature Genetics 41:753-761, 2009; and U.S. Pat. Nos. 6,489,458; 7,148,203; and 7,160,682; US Publication Nos. 2011/117072; 2004/077572; and 2006/252140. In certain embodiments, the Sleeping Beauty transposase enzyme has the sequence SEQ ID NO: 34. In particular embodiments, the Hyperactive Sleeping Beauty (SB100x) transposase enzyme has the sequence SEQ ID NO: 35.

Systematic mutagenesis studies have been undertaken to increase the activity of the SB transposase. For example, Yant et al., undertook the systematic exchange of the N-terminal 95 AA of the SB transposase for alanine (Mol. Cell Biol. 24: 9239-9247, 2004). Ten of these substitutions caused hyperactivity between 200-400% as compared to SB10 as a reference. SB16, described in Baus et al. (Mol. Therapy 12: 1148-1156, 2005) was reported to have a 16-fold activity increase as compared to SB10. Additional hyperactive SB variants are described in Zayed et al. (Molecular Therapy 9(2):292-304, 2004) and U.S. Pat. No. 9,840,696.

SB transposons need to circularize in order to transpose (Yant et al., Nature Biotechnology, 20: 999-1005, 2002). Furthermore, there is an inverse linear relationship, for transposons between 1.9 and 7.2 kb, between the length of the transposon and transposition frequency. In other words, SB transposase mediate the delivery of larger transposons less efficiently compared to smaller transposons (Geurts et al., Mol Ther., 8(1):108-17, 2003).

SB transposases transpose nucleic acid transposon payloads that are positioned between SB ITRs. Various SB ITRs are known in the art. In some embodiments, an SB ITR is a 230 bp sequence including imperfect direct repeats of 32 bp in length that serve as recognition signals for the transposase. Engineered SB ITRs are known in the art, including SB ITRs known as pT, pT2, pT3, pT2B, and pT4. In some embodiments, pT4 ITRs are used, e.g., to flank a transposon payload of the present disclosure, e.g., for transposition by an SB100x transposase.

In particular embodiments, the sequence encoding the IR(inverted repeat)/DR(direct repeat) of Sleeping Beauty includes SEQ ID NO: 38. In particular embodiments, the sequence encoding the IR/DR and chromosomal sequence of Sleeping Beauty includes SEQ ID NOs: 39 or 40. In particular embodiments, the IR/DR encoding sequence of Sleeping Beauty includes SEQ ID NO: 41. In particular embodiments, the sequence encoding the IR/DR and chromosomal sequence of Sleeping Beauty includes SEQ ID NO: 42. In particular embodiments, the sequence encoding the IR/DR of Sleeping Beauty includes SEQ ID NO: 43.

In particular embodiments, landing pads additionally include a promoter. The promoter can be inducible or constitutive.

Examples of inducible promoter systems that can be used in the systems and methods of the present disclosure include: lac operon (Brown et al. (1987) Cell 49: 603-612; Hu and Davidson (1987) Cell 48: 555-566); tetracycline (Tet) (or derivative doxycycline)-inducible systems (Tet-On and Tet-Off) (Gossen et al. (1995) Science 268: 1766-1769; Baron et al. (1997) Nucleic Acids Res 25: 2723-2739; Blau and Rossi (1999) Proc Natl Acad Sci USA 96: 797-799); mifepristone-inducible systems (GeneSwitch) (Burcin et al. (1999) Proc. Natl. Acad. Sci. USA 96(2): 355-360; Wang et al. (1994) Proc. Natl. Acad. Sci. USA 91(17): 8180-8184); ecdysone-regulated system (Galimi et al. (2005) Blood 105(6): 2400-2402); streptogramin-adjustable expression system derived from Streptomyces coelicolor (Mitta et al. (2004) Nucleic Acids Res 32(12): e106); gaseous acetaldehyde-inducible expression system derived from Aspergillus nidulans (Hartenbach S & Fussenegger M (2005) J Biotechnol 120(1): 83-98); and cumate-inducible systems (U.S. Pat. No. 7,745,592; Mullick et al. (2006) BMC Biotechnology 6:43).

Examples of constitutive promoters include CMV (Karasuyama et al. 1989. J. Exp. Med. 169:13), ubiquitin, beta-actin (Gunning et al. 1989. Proc. Natl. Acad. Sci. USA 84:4831-4835) pgk (see, for example, Adra et al. 1987. Gene 60:65-74; Singer-Sam et al. 1984. Gene 32:409-417; and Dobson et al. 1982. Nucleic Acids Res. 10:2635-2637), and common EF1α long and short constitutive promoters.

Particular embodiments can utilize minBglobin, CMV, minRho, the SV40 immediately early promoter, the Hsp68 minimal promoter (proHSP68), or the Rous Sarcoma Virus (RSV) long-terminal repeat (LTR) promoter.

Particular embodiments utilize the Tre3g promoter. The TRE3G promoter is a part of the Tet-On tetracycline-inducible expression system which provides for very low basal expression and high maximal expression after induction (Loew et. al., 2010, BMC Biotechnol. 10:81). It includes 7 repeats of a 19 bp tet operator sequence located upstream of a minimal CMV promoter. In the presence of Dox, Tet-On 3G binds specifically to P_{TRE3G} and activates transcription of the downstream gene of interest (GOI). P_{TRE3G} lacks binding sites for endogenous mammalian transcription factors, so it is virtually silent in the absence of induction. In particular embodiments, the Tre3g promoter includes the TRE3GV promoter or the TRE3GS promoter.

In particular embodiments, the TRE3GV promoter includes the sequence:

(SEQ ID NO: 44)
TTACTCCCTATCAGTGATAGAGAACGTATGAAGAGTTTACTCCCTATCAGTGATAGAGAACG
TATGCAGACTTTACTCCCTATCAGTGATAGAGAACGTATAAGGAGTTTACTCCCTATCAGTG
ATAGAGAACGTATGACCAGTTTACTCCCTATCAGTGATAGAGAACGTATCTACAGTTTACTC
CCTATCAGTGATAGAGAACGTATATCCAGTTTACTCCCTATCAGTGATAGAGAACGTATGTC
GAGGTAGGCGTGTACGGTGGGCGCCTATAAAAGCAGAGCTCGTTTAGTGAACCGTCAGAT
CGCCTGGAGCAATTCCACAACACTTTTGTCTTATACTT

In particular embodiments, the TRE3GS promoter includes the sequence

(SEQ ID NO: 45)
GAGTTTACTCCCTATCAGTGATAGAGAACGTATGAAGAGTTTACTCCCTATCAGTGATAGAG
AACGTATGCAGACTTTACTCCCTATCAGTGATAGAGAACGTATAAGGAGTTTACTCCCTATC
AGTGATAGAGAACGTATGACCAGTTTACTCCCTATCAGTGATAGAGAACGTATCTACAGTTT
ACTCCCTATCAGTGATAGAGAACGTATATCCAGTTTACTCCCTATCAGTGATAGAGAACGTA
TAAGCTTTGCTTATGTAAACCAGGGCGCCTATAAAAGAGTGCTGATTTTTTGAGTAAACTTC
AATTCCACAACACTTTTGTCTTATACCAACTTTCCGTACCACTTCCTACCCTCGTAAA.

In particular embodiments, the TRE3GV promoter within the landing pad includes the sequence as set forth in SEQ ID NO: 44. In particular embodiments, the TRE3GS promoter within the viral vector includes the sequence as set forth in SEQ ID NO: 45.

Particular embodiments utilize the CMV promoter. In particular embodiments, the CMV promoter includes the sequence as set forth in SEQ ID NO: 2.

In particular embodiments, landing pads include a selection marker, also referred to herein as a reporter. A reporter allows for determining the appropriate integration of a construct into the genome of a cell. A “reporter gene” is a gene whose expression confers a phenotype upon a cell that can be easily identified and measured. In some embodiments, a reporter gene encodes a fluorescent protein gene.

In particular embodiments, the reporter is green fluorescent protein (GFP). However, as is understood by those or ordinary skill in the art, any appropriate reporter can be used. Additional examples include blue fluorescent proteins (e.g. eBFP, eBFP2, Azurite, mKalamal, GFPuv, Sapphire, T-sapphire); cyan fluorescent proteins (e.g. eCFP, Cerulean, CyPet, AmCyanl, Midoriishi-Cyan); additional green fluorescent proteins (e.g. GFP-2, tagGFP, turboGFP, eGFP, Emerald, Azami Green, Monomeric Azami Green, CopGFP, AceGFP, ZsGreen); orange fluorescent proteins (mOrange, mKO, Kusabira-Orange, Monomeric Kusabira-Orange, mTangerine, tdTomato); red fluorescent proteins (mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, Jred); yellow fluorescent proteins (e.g., YFP, eYFP, Citrine, Venus, YPet, PhiYFP, ZsYellowl); and any other suitable fluorescent proteins, including, for example, firefly luciferase. In particular embodiments, the reporter can include any cell surface displayed marker that can be detected with an antibody that binds to that marker and allows sorting of cells that have the marker. In particular embodiments, the reporter can include the magnetic sortable marker streptavidin binding peptide (SBP) displayed at the cell surface by a truncated Low Affinity Nerve Growth Receptor (LNGFRF) and one-step selection with streptavidin-conjugated magnetic beads (Matheson et al. (2014) PloS one 9(10): e111437). In particular embodiments, the selection marker includes ZsGreen which is encoded by the sequence as set forth in SEQ ID NO: 4. In particular embodiments, the selection marker includes mCherry.

In some embodiments, the reporter gene includes a selection gene. A “selection gene” refers to the use of a gene which encodes an enzymatic activity that confers the ability to grow in medium lacking what would otherwise be an essential nutrient; in addition, a selection gene may confer resistance to an antibiotic or drug upon the cell in which the selection gene is expressed. A selection gene may be used to confer a particular phenotype upon a host cell. When a host cell must express a selection gene to grow in selective medium, the gene is said to be a positive selection gene. A selection gene can also be used to select against host cells containing a particular gene; a selection gene used in this manner is referred to as a negative selection gene. In exemplary embodiments the selection gene is placed downstream of KRAB sequence following the repressor protein, and also downstream of an internal ribosome entry site (IRES) sequence. In exemplary embodiments, the selection gene is an antibiotic resistance gene, including for example a gene that confers resistance to gentamycin, thymidine kinase, ampicillin, puromycin, and/or kanamycin.

Particular embodiments can also utilize cerulenin resistance genes (e.g., fas2m, PDR4; Inokoshi et al., Biochemistry 64: 660, 1992; Hussain et al., Gene 101: 149, 1991); copper resistance genes (CUP1; Marin et al., Proc. Natl. Acad. Sci. USA. 81: 337, 1984); and geneticin resistance gene (G418r) as reporters. However, one of ordinary skill in the art will appreciate that the systems and methods described herein can be made, performed, and used without a reporter. Absence of a reporter, however, decreases the efficiency of the disclosed systems and methods.

Additional useful selection markers include β-galactosidase (β-gal) and β-glucuronidase (GUS) (see, e.g., European Patent Publication EP2423316). These reporter proteins function by hydrolyzing a secondary marker molecule (e.g., a β-galactoside or a β-glucuronide). Thus, it will be understood that methods and systems that employ one of these marker proteins will also involve providing the compound(s) needed to produce a detectable reaction product. Assays for detecting β-gal or GUS activity are well known in the art.

In particular embodiments it may be appropriate to use auxotrophic markers as reporters. Exemplary auxotrophic markers include methionine auxotrophic markers (e.g., met1, met2, met3, met4, met5, met6, met7, met8, met10, met13, met14 or met20); tyrosine auxotrophic markers (e.g., tyr1 or isoleucine); valine auxotrophic markers (e.g., ilv1, ilv2, ilv3 or ilv5); phenylalanine auxotrophic markers (e.g., pha2); glutamic acid auxotrophic markers (e.g., glu3); threonine auxotrophic markers (e.g., thr1 or thr4); aspartic acid auxotrophic markers (e.g., asp1 or asp5); serine auxotrophic markers (e.g., ser1 or ser2); arginine auxotrophic markers (e.g., arg1, arg3, arg4, arg5, arg8, arg9, arg80, arg81, arg82 or arg84); uracil auxotrophic markers (e.g., ura1, ura2, ura3, ura4, ura5 or ura6); adenine auxotrophic markers (e.g., ade1, ade2, ade3, ade4, ade5, ade6, ade8, ade9, ade12 or ade15); lysine auxotrophic markers (e.g., lys1, lys2, lys4, lys5, lys7, lys9, lys11, lys13 or lys14); tryptophan auxotrophic markers (e.g., trp1, trp2, trp3, trp4 or trp5); leucine auxotrophic markers (e.g., leu1, leu2, leu3, leu4 or leu5); and histidine auxotrophic markers (e.g., his1, his2, his3, his4, his5, his6, his7 or his8).

In particular embodiments, landing pads can include insulator sequences. Insulator sequences can contribute to protecting expression of proteins from integration site effects, which can be mediated by cis-acting elements present in genomic DNA and lead to deregulated expression of transferred sequences (i.e., position effect; see, e.g., Burgess-Beusse et al., PNAS., USA, 99:16433, 2002; and Zhan et al., Hum. Genet., 109:471, 2001). An insulator sequence insulates a transferred sequence from unintended “read through” transcription from another promoter located upstream to the transferred sequence. The transferred sequence can be flanked at the 5′ and/or the 3′ end by an insulator. Suitable insulators include transcription terminators or nucleic acids forming a structure that sterically hinder an unintended read through transcription from an upstream promoter. Example insulators include the chicken β-globin insulator (see Chung et al., Cell 74:505, 1993; Chung et al., PNAS USA 94:575, 1997; and Bell et al., Cell 98:387, 1999), SP10 insulator (Abhyankar et al., JBC 282:36143, 2007), a Scaffold or Matrix Attachment Region (S/MAR) (e.g., MAR X_S29), a Stabilizing Anti Repressor (STAR) element (e.g., STAR40), a D4Z4 insulator, A Ubiquitous Chromatin Opening Element (UCOE element) (e.g., aHNRPA2B1-CBX3 locus (A2UCOE), 3′UCOE, or SRF-UCOE), or other small CTCF recognition sequences that function as enhancer blocking insulators (Liu et al., Nature Biotechnology, 33:198, 2015). In particular embodiments, the insulator includes a cHS4 insulator. In particular embodiments, the cHS4 insulator includes the sequence as set forth in SEQ ID NO: 1.

In certain examples, landing pads can include homology arms to facilitate integration of the landing pad into the selected locus. Particular embodiment can utilize homology arms with 25, 50, 100, or 200 nucleotides (nt), or more than 200 nt of sequence homology between a homology-directed repair template and a targeted genomic sequence (or any integral value between 10 and 200 nucleotides, or more). In particular embodiments, homology arms are 40-1000 nt in length. In particular embodiments, homology arms 500-2500 base pairs, 700-2000 base pairs, or 800-1800 base pairs. In particular embodiments, homology arms include at least 800 base pairs or at least 850 base pairs. The length of homology arms can also be symmetric or asymmetric.

Particular embodiment can utilize first and/or second homology arms each including at least 25, 50, 100, 200, 400, 600, 800, 1,000, 1,200, 1,400, 1,600, 1,800, 2,000, 2,500, or 3,000 nucleotides or more, having sequence identity or homology with a corresponding fragment of a target genome. In some embodiments, first and/or second homology arms each include a number of nucleotides having sequence identity or homology with a corresponding fragment of a target genome that has a lower bound of 25, 50, 100, 200, 400, 600, 800, 1,000, 1,200, 1,400, 1,600, or 1,800 nucleotides and an upper bound of 1,000, 1,200, 1,400, 1,600, 1,800, 2,000, 2,500, or 3,000 nucleotides. In some embodiments, first and/or second homology arms each include a number of nucleotides having sequence identity or homology with a corresponding fragment of a target genome that is between 40 and 1,000 nucleotides, between 500 and 2,500 nucleotides, between 700 and 2,000 nucleotides, or between 800 and 1800 nucleotides, or that has a length of at least 800 nucleotides or at least 850 nucleotides. First and second homology arms can have same, similar, or different lengths.

For additional information regarding homology arms, see Richardson et al., Nat Biotechnol. 34(3):339-44, 2016.

Particular embodiments include selecting cells that include the landing pad using the selection marker to obtain an isolated population of the mammalian cells that include the landing pad. Once selected cells that include the landing pad are selected, the method further includes introducing into the selected cells a viral vector including or encoding a barcode and a protein of interest at the landing pad.

(iii) Viral Vectors for Integration into Landing Pads. The term vector refers to a nucleic acid molecule capable of transferring or transporting another nucleic acid molecule into a cell. The transferred nucleic acid is generally linked to, e.g., inserted into, the vector nucleic acid molecule. A vector may include sequences that direct autonomous replication in a cell, or may include sequences that permit integration into host cell DNA. Useful vectors include, for example, viral vectors.

Viral vector is widely used to refer to a nucleic acid molecule that includes virus-derived nucleic acid elements that facilitate transfer and expression of non-native nucleic acid molecules within a cell. The term adeno-associated viral vector refers to a viral vector or plasmid containing structural and functional genetic elements, or portions thereof, that are primarily derived from AAV. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, that are primarily derived from a lentivirus, and so on. The term “hybrid vector” refers to a vector including structural and/or functional genetic elements from more than one virus type.

Viral vectors used within the systems and methods disclosed herein can use any retroviral vector backbone, as long as the retroviral vector is able to be integrated into the genome of a cell at a landing pad site. A retroviral vector can integrate into a host cell genome with the help of integrase binding sites. Enzymes of the host can be used for replication of the integrated retroviral vector. The retroviral vectors can be derived from members of the Retroviridae family. The Retroviridae family includes three groups: the spumaviruses (or foamy viruses) such as the human foamy virus (HFV); the lentiviruses, as well as visna virus of sheep; and the oncoviruses. The term “lentivirus” includes a genus of viruses containing reverse transcriptase. The lentiviruses include the “immunodeficiency viruses” which include human immunodeficiency virus (HIV) type 1 and type 2 (HIV-1 and HIV-2) and simian immunodeficiency virus (SIV). The oncoviruses have historically been further subdivided into groups A, B, C and D on the basis of particle morphology, as seen under the electron microscope during viral maturation. The C-type viruses are the most commonly studied and include many of the avian and murine leukemia viruses (MLV). Bovine leukemia virus (BLV), and the human T-cell leukemia viruses types I and II (HTLV-I/II) are similarly classified as C-type particles.

Retroviruses have been classified in various ways but the nomenclature has been standardized (see ICTVdB—The Universal Virus Database, v 4 on the World Wide Web (www) at ncbi.nlm.nih.gov/ICTVdb/ICTVdB/ and the text book “Retroviruses” Eds Coffin, Hughs and Varmus, Cold Spring Harbor Press 1997).

Retroviruses are defined by the way in which they replicate their genetic material. During replication the RNA is converted into DNA. Following infection of the cell a double-stranded molecule of DNA is generated from the two molecules of RNA which are carried in the viral particle by the molecular process known as reverse transcription. The DNA form becomes covalently integrated in the host cell genome as a provirus, from which viral RNAs are expressed with the aid of cellular and/or viral factors. The expressed viral RNAs are packaged into particles and released as infectious viral particles.

The retrovirus particle is composed of two identical RNA molecules. Each wild-type genome has a positive sense, single-stranded RNA molecule, which is capped at the 5′ end and polyadenylated at the 3′ tail. The diploid virus particle contains the two RNA strands complexed with gag proteins, viral enzymes (pol gene products) and host tRNA molecules within a ‘core’ structure of gag proteins. Surrounding and protecting this capsid is a lipid bilayer, derived from host cell membranes and containing viral envelope (env) proteins. The env proteins bind to a cellular receptor for the virus and the particle typically enters the host cell via receptor-mediated endocytosis and/or membrane fusion.

After the outer envelope is shed, the viral RNA is copied into DNA by reverse transcription. This is catalyzed by the reverse transcriptase enzyme encoded by the pol region and uses the host cell tRNA packaged into the virion as a primer for DNA synthesis. In this way the RNA genome is converted into the more complex DNA genome.

The double-stranded linear DNA produced by reverse transcription may, or may not, have to be circularized in the nucleus. The provirus now has two identical repeats at either end, known as the long terminal repeats (LTR). The termini of the two LTR sequences produces the site recognized by a pol product—the integrase protein—which catalyzes integration, such that the provirus is always joined to host DNA two base pairs (bp) from the ends of the LTRs. A duplication of cellular sequences is seen at the ends of both LTRs, reminiscent of the integration pattern of transposable genetic elements. Retroviruses can integrate their DNAs at many sites in host DNA, but different retroviruses have different integration site preferences.

Transcription, RNA splicing and translation of the integrated viral DNA is mediated by host cell proteins. Variously spliced transcripts are generated. In the case of the human retroviruses HIV-1/2 and HTLV-I/II, viral proteins are also used to regulate gene expression. The interplay between cellular and viral factors is a factor in the control of virus latency and the temporal sequence in which viral genes are expressed.

Key to the current disclosure is that viral vectors are designed with features that direct integration into landing pads. Viral vectors that are integrated within landing pads include at least an integrase attachment site, a barcode, and a sequence encoding a protein of interest.

In particular embodiments, the viral vector is flanked on both the 5′ and 3′ ends by transposon-specific inverted terminal repeats (ITR). As described herein, it is the use of transposon ITRs (in combination with a corresponding transposase) that allow for the specific integration of the viral vector into the genome at a landing pad within the target cell.

The methods for producing the lentiviral packaging vector-containing mammalian cell further include culturing the transfected mammalian cell to allow for integration of the desired nucleic acids (i.e., the viral vector) into the genome of the cell, followed by isolating the lentiviral packaging vector-containing mammalian cell.

Integrase attachment sites and integrase/recombinase systems are described elsewhere herein. An integrase that is introduced into or already present in the cells recognizes the integrase recognition sites and removes at least the segment of the landing pad, such that a segment of the landing pad is replaced by the viral vector by homologous recombination of the viral vector into the selected locus. The amount of viral vector and integrase introduced into the cell with an integrated landing pad is sufficient to provide for the desired excision and insertion of the viral vector into the landing pad within the cell genome. Particular embodiments include a 1:1; 1:2; or 1:3 ratio of viral vector to transposase/recombinase.

The methods result in stable integration of the viral vector into the target cell genome at the landing pad site. By stable integration it is meant that the viral vector remains present in the target cell genome for more than a transient period of time and is passed on a part of the chromosomal genetic material to the progeny of the target cell.

In particular embodiments, the method further includes selecting cells with an integrated a viral vector such that only mammalian cells that contain the landing pad and the viral vector are utilized in research libraries.

In particular embodiments, the barcode of an integrated viral vector is 18-nucleotides in length. In particular embodiments, because there are 4¹⁸-7¹⁰ different 18-nucleotide sequences, virtually every variant can have a unique barcode. The barcode can be any appropriate length and composition that does not negatively affect fitness of an encoded protein of interest. In particular embodiments, the length of the barcode is based upon the size of the deep mutation scanning library. If more distinct barcodes are needed, then barcodes of greater length can be used. If less distinct barcodes are needed, then barcodes of lesser length can be used. In particular embodiments, the barcode can be 5-100 nucleotides in length. In particular embodiments, the barcode can be 10-80 nucleotides in length. In particular embodiments, the barcode can be 10-50 nucleotides in length. In particular embodiments, the barcode can be 8-30 nucleotides in length. In particular embodiments, the barcode can be 12-24 nucleotides in length. In particular embodiments, the barcode can be 16-20 nucleotides in length. In particular embodiments, the barcode can be 3 nucleotides in length, 4 nucleotides in length, 5 nucleotides in length, 6 nucleotides in length, 7 nucleotides in length, 8 nucleotides in length, 9 nucleotides in length, 10 nucleotides in length, 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, 29 nucleotides in length, 30 nucleotides in length, 31 nucleotides in length, 32 nucleotides in length, 33 nucleotides in length, 34 nucleotides in length, 35 nucleotides in length, 36 nucleotides in length, 37 nucleotides in length, 38 nucleotides in length, 39 nucleotides in length, 40 nucleotides in length, or more.

In particular embodiments, the barcode can be positioned anywhere within the viral vector that is integrated into a cell's genome. In particular embodiments, the barcode can be positioned upstream (5′) of the gene encoding a protein of interest. In particular embodiments, the barcode can be positioned within the gene encoding a protein of interest. In particular embodiments, the barcode can be positioned downstream (3′) of the gene encoding a protein of interest. In particular embodiments, the barcode can be positioned after the stop codon. In particular embodiments, the barcode can include random nucleotides flanked by priming sequences (e.g., Illumina priming sequences).

In particular embodiments, long-read PacBio sequencing can be performed to link barcodes to the encoded protein of interest. Linking barcodes to encoded proteins of interest allows for the use of short-read Illumina sequencing of the barcode to obtain the full encoded proteins of interest in subsequent experiments. This genotype-phenotype linkage through sequencing is performed before a viral vector is integrated into a landing pad within a cell.

Viral vectors used within the libraries disclosed herein can also include sequences to facilitate sequencing. In particular embodiments, sequences to facilitate sequencing can include Illumina P5 and P7 sequences and other adapter sequences included in Illumina Adapter Sequences, Document #1000000002694 v06, February 2018 (Illumina, San Diego, CA).

Proteins of interest can be any protein. Exemplary proteins of interest include viral proteins (e.g., viral entry proteins), receptors (e.g., T cell receptors, B cell receptors, hormone receptors), and receptor ligands (e.g., T cell receptor antigens, B cell receptor antigens, hormones, cytokines, etc.).

In particular embodiments, viral entry proteins [virus (entry protein)] include: Chikungunya (E1 Env and E2 Env), Ebola glycoprotein (EBOV GP), Hendra (F glycoprotein and G glycoprotein), hepatitis B (large (L), middle (M), and small (S)), hepatitis C (glycoprotein E1 and glycoprotein E2), HIV envelope (Env), influenza hemagglutinin (HA), Lassa virus envelope glycoprotein (GPC), measles (hemagglutinin glycoprotein (H) and fusion glycoprotein F0 (F)), MERS-CoV (Spike (S)), Nipah (fusion glycoprotein F0 (F) and glycoprotein G), Rabies virus glycoprotein (RABV G), RSV (fusion glycoprotein F0 (F) and glycoprotein G), and SARS-CoV (Spike (S)), among many others.

In particular embodiments, a protein of interest is part of a deep mutational scanning (DMS) library. In certain examples a DMS library includes variants with 19 possible amino acid substitutions at each amino acid position and all possible codons of the associated 63 codons at each amino acid position. In particular embodiments, a deep mutational scanning library includes variants with every possible codon substitution at every amino acid position in a gene of interest with one codon substitution per library member. In particular embodiments, a deep mutational scanning library includes variants with one, two, or three nucleotide changes for each codon at every amino acid position in a gene of interest with one codon substitution per library member. In particular embodiments, a deep mutational scanning library includes variants with one, two, or three nucleotide changes for each codon at two amino acid positions, at three amino acid positions, at four amino acid positions, at five amino acid positions, at six amino acid positions, at seven amino acid positions, at eight amino acid positions, at nine amino acid positions, at ten amino acid positions, etc., up to at all amino acid positions, in a gene of interest with one codon substitution per library member. In particular embodiments, the start codon is not mutagenized. In particular embodiments, the start codon is Met.

In particular embodiments, a deep mutational scanning library includes variants with one, two, or three nucleotide changes for each codon at every amino acid position in a gene of interest with more than one codon substitution, more than two codon substitutions, more than three codon substitutions, more than four codon substitutions, or more than five codon substitutions, per library member. In particular embodiments, a deep mutational scanning library includes variants with one, two, or three nucleotide changes for each codon at every amino acid position in a gene of interest with up to all codon substitutions per library member. In particular embodiments, 20% of library members can be wildtype, 35% can be single mutants, and 45% can be multiple mutants. Multiple mutants can be advantageous, and the sequencing required by the systems and methods disclosed herein is so efficient that using 20% of reads on wildtype is not a problem. Additionally, there are alternative (more complex) mutagenesis methods that give a larger proportion of single amino acid mutants (see, e.g., Kitzman, et al. (2015) Nature Methods 12: 203-206; Firnberg & Ostermeier (2012) PLoS One 7: e52031; Jain & Varadarajan (2014) Analytical Biochemistry 449: 90-98; and Wrenbeck, et al. (2016) Nature Methods 13: 928).

In particular embodiments, a deep mutational scanning library includes or encodes all possible amino acids at all positions of a protein, and each protein of interest is encoded by more than one variant nucleotide sequence. In particular embodiments, a deep mutational scanning library includes or encodes all possible amino acids at all positions of a protein, and each protein of interest is encoded by one nucleotide sequence. In particular embodiments, a deep mutational scanning library includes or encodes all possible amino acids at less than all positions of a protein, for example at 10%, 20%, 30%, 40%, 50%, 60%, 70%, 80%, 90%, 95%, 96%, 97%, 98% or 99% of positions. In particular embodiments, a deep mutational scanning library includes or encodes less than all possible amino acids (for example 10%, 20%, 30%, 40%, 50%, 60%, 70%, 80%, 90%, 95%, 96%, 97%, 98% or 99% of potential amino acids) at all positions of a protein. In particular embodiments, a deep mutational scanning library includes or encodes less than all possible amino acids (for example 10%, 20%, 30%, 40%, 50%, 60%, 70%, 80%, 90%, 95%, 96%, 97%, 98% or 99% of potential amino acids) at less than all positions of a protein, for example at 10%, 20%, 30%, 40%, 50%, 60%, 70%, 80%, 90%, 95%, 96%, 97%, 98% or 99% of positions. In particular embodiments, a deep mutational scanning library including a set of variant nucleotide sequences can collectively encode protein variants including at least a particular number of amino acid substitutions at at least a particular percentage of amino acid positions. “Collectively encode” takes into account all amino acid substitutions at all amino acid positions encoded by all the variant nucleotide sequences in total in a deep mutational scanning library.

In particular embodiments, a codon-mutant library can be generated by PCR, primer-based mutagenesis, as described in US2016/0145603. In particular embodiments, a codon-mutant library can be synthetically constructed by and obtained from a synthetic DNA company such as Twist Bioscience (San Francisco, CA). In particular embodiments, methods to generate a codon-mutant library include: nicking mutagenesis as described in Wrenbeck et al. (2016) Nature Methods 13: 928-930 and Wrenbeck et al. (2016) Protocol Exchange doi:10.1038/protex.2016.061; PFunkel (Firnberg & Ostermeier (2012) PLoS ONE 7(12): e52031); massively parallel single-amino-acid mutagenesis using microarray-programmed oligonucleotides (Kitzman et al. (2015) Nature Methods 12: 203-206); and saturation editing of genomic regions with CRISPR-Cas9 (Findlay et al. (2014) Nature 513(7516): 120-123).

Viral vectors used within the libraries disclosed herein can also encode a linker. In particular embodiments, the following linkers can be used:

| | | Associated Protein |
|---|---|---|
| Linker | Exemplary Encoding Sequence | Sequence |
| thosea asigna | (GGCAGCGGC)GAAGGCCGCGGCAGCCTGCT | (GSG)EGRGSLLTC |
| virus T2A | GACCTGCGGCGATGTGGAAGAAAACCCGGGC | GDVEENPGP (SEQ |
| | CCG (SEQ ID NO: 46) | ID NO: 47) |
| porcine | (GGCAGCGGC)GCGACCAACTTTAGCCTGCTG | (GSG)ATNFSLLKQA |
| teschovirus-1 | AAACAGGCGGGCGATGTGGAAGAAAACCCGG | GDVEENPGP (SEQ |
| P2A | GCCCG (SEQ ID NO: 48) | ID NO: 49) |
| equine rhinitis A | (GGCAGCGGC)CAGTGCACCAACTATGCGCTG | (GSG)QCTNYALLKL |
| virus E2A | CTGAAACTGGCGGGCGATGTGGAAAGCAACC | AGDVESNPGP |
| | CGGGCCCG (SEQ ID NO: 50) | (SEQ ID NO: 51) |
| foot-and-mouth | (GGCAGCGGC)GTGAAACAGACCCTGAACTTT | (GSG)VKQTLNFDLL |
| disease virus F2A | GATCTGCTGAAACTGGCGGGCGATGTGGAAA | KLAGDVESNPGP |
| | GCAACCCGGGCCCG (SEQ ID NO: 52) | (SEQ ID NO: 53) |
| equine rhinitis B | GAAGCAACTTTGTCTACCATTCTGTCTGAGGG | EATLSTILSEGATNF |
| virus 12A | TGCCACA | SLLKLAGDVELNPG |
| | AATTTTTCTTTGTTGAAGTTAGCAGGGGATGTT | P (SEQ ID NO: 55) |
| | GAACTTAACCCCGGCCCA (SEQ ID NO: 54) | |
| Saffold virus 2A | TTCACTGATTTTTTCAAAGCCGTTAGAGACTAT | FTDFFKAVRDYHAS |
| | CATGCTTCTTATTACAAACAGAGACTTCAACAT | YYKQRLQHDVETN |
| | GACGTTGAAACAAACCCTGGCCCT (SEQ ID | PGP (SEQ ID NO: |
| | NO: 56) | 57 |
| Ljungan virus 2A | TACTTTAATATAATGCACAGTGATGAAATGGAT | YFNIMHSDEMDFAG |
| | TTTGCCGGGGGGAAATTTTTGAATCAATGTGG | GKFLNQCGDVETN |
| | TGATGTGGAAACTAACCCAGGCCCT (SEQ ID | PGP (SEQ ID NO: |
| | NO: 58) | 59) |
| infectious | CCCTCAATTGGTAATGTCGCGCGGACTCTGAC | PSIGNVARTLTRAEI |
| flacherie virus 2A | GAGGGCGGAGATTGAGGATGAATTGATTCGT | EDELIRAGIESNPGP |
| | GCAGGAATTGAATCAAATCCTGGACCT (SEQ | (SEQ ID NO: 61) |
| | ID NO: 60) | |
| Perina nuda | GGACAAAGGACGACTGAACAGATAGTTACGG | GQRTTEQIVTAQG |
| picorna-like virus | CCCAGGGGTGGGTTCCGGATTTGACTGTGGA | WVPDLTVDGDVES |
| 2A₁ | TGGAGATGTTGAGTCAAATCCCGGACCC (SEQ | NPGP (SEQ ID NO: |
| | ID NO: 62) | 63 |
| Perina nuda | ACGCGTGGTGGTTTACGACGGCAAAATATTAT | TRGGLRRQNIIGGG |
| picorna-like virus | TGGTGGTGGGCAGAAGGATTTGACACAAGAT | QKDLTQDGDIESNP |
| 2A₂ | GGTGACATCGAGTCGAATCCTGGGCCC (SEQ | GP (SEQ ID NO: 65) |
| | ID NO: 64) | |
| Ectropis obliqua | GGACAACGGACAACTGAGCAGATCGTGACTG | GQRTTEQIVTAQG |
| picorna-like virus | CACAAGGTTGGGCCCCGGATTTGACACAGGA | WAPDLTQDGDVES |
| 2A₁ | TGGAGATGTAGAGTCAAACCCCGGCCCC | NPGP (SEQ ID NO: |
| | (SEQ ID NO: 66) | 67) |
| Ectropis obliqua | ACACGTGGTGGTTTACAGCGTCAAAACATTAT | TRGGLQRQNIIGGG |
| picorna-like virus | TGGTGGTGGCCAAAGGGATCTGACTCAAGAT | QRDLTQDGDIESNP |
| 2A₂ | GGCGACATCGAGTCGAACCCCGGCCCA (SEQ | GP (SEQ ID NO: 69) |
| | ID NO: 68) | |
| Drosophila C | CAAGGCATCGGTAAGAAGAATCCGAAACAGG | QGIGKKNPKQEAAR |
| virus 2A | AAGCTGCACGTCAGATGTTGCTCTTGTTATCA | QMLLLLSGDVETNP |
| | GGAGATGTTGAGACTAACCCTGGACCC (SEQ | GP (SEQ ID NO: 71) |
| | ID NO: 70) | |
| acute bee | ACTGGTTTTTTAAACAAGTTATATCATTGTGGC | TGFLNKLYHCGSW |
| paralysis virus 2A | TCATGGACTGACATATTGTTGTTGTTGTCTGG | TDILLLLSGDVETNP |
| | AGATGTAGAAACCAATCCAGGACCT (SEQ ID | GP (SEQ ID NO: 73) |
| | NO: 72) | |
| Euprosterna | CGACGATTGCCGGAGTCCGCCCAGCTCCCCC | RRLPESAQLPQGA |
| elaeasa virus 2A | AAGGGGCGGGGCGCGGAAGTCTGGTAACAT | GRGSLVTCGDVEE |
| | GTGGCGACGTGGAGGAGAATCCAGGGCCC | NPGP (SEQ ID NO: |
| | (SEQ ID NO: 74) | 75) |
| Providence virus | TTGGAGATGAAGGAGTCTAATAGTGGTTACGT | LEMKESNSGYVVG |
| 2A₁ | AGTCGGTGACCGGGGGTCTCTTCTCACTTGT | DRGSLLTCGDVES |
| | GGGGACGTTGAATCCAACCCTGGACCC (SEQ | NPGP (SEQ ID NO: |
| | ID NO: 76) | 77) |
| Providence virus | ACGCTTATGGGGAACATCATGACACTTGCAGG | TLMGNIMTLAGSGG |
| 2A₃ | GTCAGGTGGTCGGGGAAGCTTGCTGACCGCA | RGSLLTAGDVEKNP |
| | GGCGATGTTGAAAAGAACCCTGGGCCC (SEQ | GP (SEQ ID NO: 79) |
| | ID NO: 78) | |
| Bombyx mori | AGAACAGCGTTCGATTTCCAGCAGGACGTTTT | RTAFDFQQDVFRS |
| cypovirus-1 2A | TCGCTCTAATTATGACCTACTAAAGTTGTGCG | NYDLLKLCGDIESN |
| | GTGATATCGAGTCTAATCCTGGACCTGTTAC | PGP (SEQ ID NO: |
| | (SEQ ID NO: 80) | 81) |
| Operophtera | ATCCATGCTAATGATTATCAGATGGCTGTGTTT | IHANDYQMAVFKSN |
| brumata | AAATCAAATTATGATTTGCTGAAGTTATGCGG | YDLLKLCGDVESNP |
| cypovirus-18 2A | GGACGTGGAATCAAATCCTGGCCCT (SEQ ID | GP (SEQ ID NO: 83) |
| | NO: 82) | |
| new adult | TTCTTCGATTCGGTTTGGGTGTACCACTTGGC | FFDSVWVYHLANS |
| diarrhea virus 2A | AAACAGCTCTTGGGTTCGAGATTTAACTAGAG | SWVRDLTRECIESN |
| | AATGCATTGAATCTAACCCTGGACCA (SEQ ID | PGP (SEQ ID NO: |
| | NO: 84) | 85) |

In particular embodiments, a 2A peptide linker including a consensus motif DXEXNPGP (SEQ ID NO: 86), where X can be any amino acid, may be used (Luke (2008) J Gen Virol 89: 1036-1042). In particular embodiments, combinations of two or more 2A peptide linkers of the same or different sequences may be used (Liu et al. (2017) Scientific Reports 7: 2193).

Viral vectors used within the libraries disclosed herein can also include an internal ribsome entry site (IRES) that allows for translation initiation in a 5′-cap independent manner. In particular embodiments, an IRES can be used to ensure that viral translation is active when host translation is inhibited. Viral vectors used within the libraries disclosed herein can also include different promoters regulating expression of a reporter gene and the gene encoding a protein of interest.

In particular embodiments, a viral vector can include a promoter, a selection marker, and/or an insulator. These optional components are described elsewhere herein.

(iv) Targeted Genetic Engineering. In particular embodiments, cells can be genetically modified at targeted sites within the genome using any targeted genetic engineering system. Particular embodiments can utilize zinc finger nucleases, homing endonucleases, Transcription Activator-Like Effector Nucleases (TALENs), megaTALs, and Clustered Regularly Interspaced Short Palindromic Repeats (CRISPR)-CRISPR associated nuclease (Cas) systems.

Zinc finger nucleases are described in, for example, US2003/0232410; US2005/0208489; US2005/0026157; US2005/0064474; US2006/0188987; US2006/0063231; US2007/0134796; US2008/015164; US2008/0131962; US2008/015996; WO2007/014275; WO2008/133938; Kim et al. (1996) Proceedings of the National Academy of Sciences of the United States of America 93: 1156-1160; Wolfe et al. (2000) Annual review of biophysics and biomolecular structure 29: 183-212; Bibikova et al. (2003) Science 300, 764; Bibikova et al. (2002) Genetics 161: 1169-1175; Miller et al. (1985) The EMBO journal 4: 1609-1614; and Miller et al. (2007) Nature biotechnology 25: 778-785.

Homing endonucleases are described in, for example, Stoddard (2011) Structure 19(1): 7-15; Arnould et al. (2006) Journal of molecular biology 355(3): 443-458; Jurica et al. (1998) Molecular cell 2(4): 469-476; US2011/0256607; WO2007/049095; WO2007/049156; WO2008/102198; WO2014/191527; and WO2014/191525.

TALENs are described in, for example, Boch et al. (2009) Science 326: 1509-1512; Moscou, & Bogdanove (2009) Science 326: 1501; Christian et al. (2010) Genetics 186: 757-761; and Miller et al. (2011) Nature biotechnology 29: 143-148. MegaTALs are described in, for example, Boissel et al. (2014) Nucleic Acids Res. 42(4): 2591-601.

CRISPR-Cas systems are described in, for example, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, and WO2017/127807.

(v) Packaging Vectors. “Packaging vectors” refer to one or more vectors that contain components necessary to produce a viral particle including a barcode and sequence encoding a protein of interest from a landing pad-integrated viral vector. The packaging vectors include expression cassettes including one or more genes and regulatory sequences to be inserted into, and ultimately expressed by, a transfected cell.

Packaging vectors can be administered as one or more helper plasmids. In particular embodiments, the packaging vector includes a lentiviral regulator of expression of virion proteins (REV) gene under control of a first promoter; a lentiviral envelope gene under control of a second promoter; and a lentiviral group specific antigen (GAG) gene and a lentiviral polymerase (POL) gene both under control of a third promoter. In other embodiments, a single promoter can control the expression of each of the REV, ENV, GAG and POL genes, or one promoter can control expression of GAG and POL, and a second promoter control the expression or REV and ENV. Other combinations are also possible and included herein.

The lentiviral regulator of expression of virion proteins (REV) is an RNA-binding protein that promotes late phase gene expression. It is also important for the transport of the unspliced or singly-spliced mRNAs, which encode viral structural proteins, from the nucleus to the cytoplasm.

The lentiviral envelope (ENV) gene, suitably a Vesicular Stomatitis Virus Glycoprotein (VSV-G) gene, encodes a polyprotein precursor which is cleaved by a cellular protease into the surface (SU) envelope glycoprotein gp120 and the transmembrane glycoprotein gp41.

In particular embodiments, alternative envelope glycoproteins (GPs) that can be used instead of VSV-G include MLV GP and feline endogenous retrovirus (RD114) GP, gibbon ape leukemia virus (GALV) Env, and variants of these. Cronin et al. (2005) Curr Gene Ther. 5(4): 387-398. In particular embodiments, GPs that can be used are derived from a family including Rhabdoviridae, Arenaviridae, Togaviridae, Filoviridae, Retroviridae, Coronaviridae, Paramyxoviridae, Flaviviridae, Orthomyxoviridae, and Baculoviridae. In particular embodiments, GPs that can be used are derived from a genus including Vesiculovirus, Lyssavirus, Arenavirus, Alphavirus, Filovirus, Alpharetrovirus, Betaretrovirus, Gammaretrovirus, Deltaretrovirus, Spumavirus, Lentivirus, Coronavirus, Respirovirus, Hepacivirus, Influenzavirus A, and nucleopolyhedrovirus. In particular embodiments, GPs that can be used are derived from a species including vesicular stomatitis virus (Indiana virus), Chandipura virus, rabies virus, Mokola virus, Lymphocytic choriomeningitis virus (LCMV), Ross River virus (RRV), Sindbis virus, Semliki Forest virus (SFV), Venezuelan equine encephalitis virus, Ebola virus Reston, Ebola virus Zaire, Marburg virus, Lassa virus, avian leukosis virus (ALV), Jaagsiekte sheep retrovirus (JSRV), MLV, GALV, RD114, human T-lymphotropic virus 1 (HTLV-1), human foamy virus, Maedi-visna virus (MVV), severe acute respiratory syndrome coronavirus (SARS-CoV), Sendai virus, Respiratory syncytial virus (RSV), human parainfluenza virus type 3, hepatitis C virus (HCV), influenza virus, fowl plague virus (FPV), and Autographa californica multiple nucleopolyhedro virus (AcMNPV).

GAG encodes a polyprotein that is translated from an unspliced mRNA which is then cleaved by the viral protease (PR) into the matrix protein, capsid, and nucleocapsid proteins. The lentiviral polymerase (POL) is expressed as a GAG-POL polyprotein as a result of ribosomal frameshifting during GAG mRNA translation, and encodes the enzymatic proteins reverse transcriptase, protease, and integrase. These three proteins are associated with the viral genome within the virion. Suitably the GAG gene is an HIV GAG gene and the POL gene is an HIV POL gene.

(vi) Uses of Genotype-Phenotype Linked Libraries. There are many uses for the genotype-phenotype linked libraries, only a portion of which are described herein. Particular embodiments utilize the libraries for mutational scanning (e.g., deep mutational scanning). These embodiments can combine functional selection with high throughput sequencing to measure the effects of mutations on protein function. In particular embodiments, a library of 104 to 105 variants of a given protein is constructed and selection for function is imposed. Under modest selection pressure, variant frequencies are perturbed according to the function of each variant. Variants harboring beneficial mutations increase in frequency, whereas variants harboring deleterious mutations decrease in frequency. In particular embodiments, high throughput sequencing can measure the frequency of each variant during the selection experiment, and a functional score can be calculated from the change in frequency over the course of the experiment. In particular embodiments, the result is a large-scale mutagenesis data set containing a functional score for each variant in the library (Fowler et al. (2014) Nature Protocols 9: 2267-2284).

In particular embodiments, variants of a protein can be exposed to different selection pressures. For example, a selection pressure can include temperature. In particular embodiments, the selection pressure is heat. Heat can include temperatures above 25° C., above 26° C., above 27° C., above 28° C., above 29° C., above 30° C., above 31° C., above 32° C., above 33° C., above 34° C., above 35° C., above 36° C., above 37° C., above 38° C., above 39° C., above 40° C., above 41° C., above 42° C., above 43° C., above 44° C., above 45° C., above 46° C., above 48° C., above 49° C., above 49° C., above 50° C., or more. In particular embodiments, heat can include temperatures from 28° C. to 70° C. In particular embodiments, heat can include temperatures from 30° C. to 65° C. In particular embodiments, heat can include temperatures above 30° C. In particular embodiments, the selection pressure is cold. Cold can include temperatures below 25° C., below 24° C., below 23° C., below 22° C., below 21° C., below 20° C., below 19° C., below 18° C., below 17° C., below 16° C., below 15° C., below 14° C., below 13° C., below 12° C., below 11° C., below 10° C., below 9° C., below 8° C., below 7° C., below 6° C., below 5° C., below 4° C., below 3° C., below 2° C., below 1° C., below 0° C., or lower. In particular embodiments, cold can include temperatures from 22° C. to 0° C. In particular embodiments, cold can include temperatures from 20° C. to 4° C. In particular embodiments, cold can include temperatures below 20° C. In particular embodiments, the selection pressure is low pH. Low pH can include pH of 6.9, 6.5, 6.0, 5.5, 5.0, 4.5, 4.0, 3.5, 3.0, 2.5, 2.0, or lower. In particular embodiments, low pH can be from pH of 6.8 to 2.0. In particular embodiments, low pH can be from pH of 6.5 to 3.0. In particular embodiments, low pH can include a pH below 6.5. In particular embodiments, the selection pressure is high pH. High pH can include pH of 7.5, 7.6, 7.7, 7.8, 7.9, 8.0, 8.5, 9.0, 9.5, 10.0, 10.5, 11.0, 11.5, 12.0, or higher. In particular embodiments, high pH can include pH of 8.0 to 14.0. In particular embodiments, high pH can include pH of 8.5 to 12.0. In particular embodiments, high pH can include a pH above 8.0. In particular embodiments, the selection pressure is a toxic agent. Toxic agents can include polar organic solvents (e.g., dimethylformamide), herbicides (e.g., glyphosate), pesticides (e.g., malathion, dichlorodiphenyltrichloroethane), salinity, ionizing radiation, and hormonally active phytochemicals (e.g., flavonoids, lignins and lignans, coumestans, or saponins).

Following expression of viral particles from the transfected cells, functional studies can be conducted to assess variant viral entry proteins. As indicated, these studies can assess the ability of different viral entry proteins to evade antibody neutralization and/or infect new species. If numerous mutations to a viral entry protein allow antibody evasion or infection of a new host species, the virus may have a higher probability of becoming a health threat. If, however, only few or very specific mutations allow antibody evasion or infection of a new host species, the virus may pose less of a threat.

In particular embodiments, libraries of the present disclosure can be used to assess the efficacy of therapeutic compounds intended to prevent, reduce, or treat the likelihood of a viral infection. Therapeutic compounds can include antiviral compounds or compositions. Examples of antiviral compounds or compositions are disclosed in, for example, U.S. Pat. Nos. 5,994,515, 6,790,611, 8,476,225, 9,259,433, US2009/0214510, US2017/0157190, WO1998/045259, WO2006/138118, WO2008/147427, WO2009/027057, WO2009/151313, WO2012/006596, WO2013/006795, WO2013/072917, WO2013/147584, WO2014/062892, and WO2015/011483; Laursen and Wilson (2013) Antiviral Res 98(3): 476-483; and Pelegrin et al. (2015) Trends in Microbiology 23(10): 653-665. Therapeutic compounds can include small molecules, derivatives of small molecules, antibodies, biologics, proteins, peptides, polynucleotides, polysaccharides, oils, solutions, and plant extracts. In particular embodiments, therapeutic compounds can include entry inhibitors and/or fusion inhibitors. Entry and fusion inhibitors can include enfuvirtide (Fuzeon® (Roche, Basel, Switzerland); a biomimetic peptide that is an HIV fusion inhibitor); AMD070 (investigational; Accession no. DB05501 from Drugbank; small molecule entry inhibitor that targets the CXCR4 receptor to block HIV infection); BMS-488043 (Hanna et al. (2011) Antimicrob Agents Chemother. 55(2): 722-728; oral small molecule HIV-1 attachment inhibitor); fozivudine tidoxil (Fogle et al. (2011) J Vet Intern Med. 25(3): 413-418; thymidine nucleoside analog is a lipid-zidovudine conjugate and member of the family of nucleoside reverse transcriptase inhibitors that decreases viremia in feline immunodeficiency virus (FIV) infected cats); aplaviroc (GSK-873,140; Nakata et al. (2005) J Virol. 79(4): 2087-2096; CCR5 entry inhibitor belonging to a class of 2,5-diketopiperazines developed for the treatment of HIV infection); leronlimab (PRO 140; fully humanized IgG4 monoclonal antibody directed against CCR5, a co-receptor that HIV uses to enter T-cells); PRO 542 (investigational; Accession no. DB05793 from Drugbank; a tetravalent CD4-immunoglobulin fusion protein that broadly neutralizes primary HIV-1 isolates); Peptide T (Pert et al. (1986) Proc Natl Acad Sci USA. 83(23): 9254-9258; a short peptide derived from the HIV envelope protein gp120 which is an HIV entry inhibitor, blocking binding and infection of viral strains which use the CCR5 receptor to infect cells); vicriviroc (SCH-D; C₂₈H₃₈F₃N₅O₂; piperazine-based CCR5 receptor antagonist with activity against HIV); ibalizumab (TNX-355; Jacobson et al. (2009) Antimicrob Agents Chemother. 53(2): 450-457; a humanized monoclonal antibody that binds CD4); and maraviroc (Selzentry® (Pfizer, New York, NY); an entry inhibitor that acts as a negative allosteric modulator of the CCR5 receptor). Other examples of viral fusion inhibitor compounds include, for example, highly sulfated polysaccharides from fucoidan or algae; calcium spirulan, nostoflan, or extract of Scoparia dulcis, or antiviral diterpene components contained therein, such as scoparic acid A, scoparic acid B, scoparic acid C, scopodiol, scopadulin, scopadulcic acid A (SDA), scopadulcic acid B (SDB), and/or scopadulcic acid C (SDC). In particular embodiments, anti-viral antibodies can include PRO 140; PRO 542; TNX-355 (ibalizumab); b12 (Burton et al. (1994) Science. 266: 1024-1027; broadly neutralizing human monoclonal IgG1 anti-gp120 antibody); polyclonal caprine antibody PEHRG214 (Verity et al. (2006) AIDS. 20(4): 505-515; antibody raised against purified HIV-associated proteins); PGT121 (Julien et al. (2013) PLoS Pathog 9(5): e1003342; broadly neutralizing antibody); 3BNC117 (Scheid et al. (2016) Nature. 535: 556-560); broadly neutralizing antibody against the CD4 binding site of HIV-1 Env); anti-RSV G protein monoclonal antibody clone 131-2G (Boyoglu-Barnum et al. (2014) J Virol. 88(18): 10569-10583); anti-CXCR4 monoclonal antibody clone 12G5 (McKnight et al. (1997) J Virol. 71(2): 1692-1696); MAB8582 (Anderson et al. (1986) Journal of Clinical Microbiology. 23(3): 475-480; anti-RSV F protein monoclonal antibody clone 102-10B); MAB8581 (Anderson et al. (1986) Journal of Clinical Microbiology. 23(3): 475-480; anti-RSV F protein monoclonal antibody clone 92-11C); MCA490 (available from Bio-Rad, Hercules, CA; anti-RSV F protein monoclonal antibody clone RSV3216 (B016)); anti-RSV F antibodies disclosed in U.S. Pat. No. 9,139,642 (104E5, 38F10, 14G3, 90D3, 56E11, 69F6); anti-Ebola virus glycoprotein (GP) monoclonal antibodies c13C6, c2G4, c4G7, and c1H3 (Tran et al. (2016) J. Virol. 90(17): 7618-7627; Murin et al. (2014) Proc Natl Acad Sci USA. 111(48): 17182-17187); LCA60 (Corti et al. (2015) PNAS. 112(33): 10473-10478; MERS-CoV-neutralizing antibody); human anti-MERS-CoV Spike protein neutralizing antibodies REGN3051 and REGN3048 (Pascal et al. (2015) PNAS. 112(28): 8738-8743); human anti-Lassa virus glycoprotein monoclonal antibodies 37.2D, 8.9F, 19.7E, 37.7H, and 12.1F (Robinson et al. (2016) Nat Commun. 7: 11544); or Hendra virus neutralizing human monoclonal antibody m102.4 (Bossart et al. (2011) Sci Transl Med. 3(105): 105ra103).

In particular embodiments, therapeutic compounds can include viral sequence integration inhibitors, proviral transcription inhibitors, protease inhibitors, and inhibitors that inhibit binding of a viral genome to one or more nucleoproteins. In particular embodiments, therapeutic compounds are compounds that are directly or indirectly effective in specifically interfering with at least one viral action including virus penetration of eukaryotic cells, virus replication in eukaryotic cells, virus assembly, virus release from infected eukaryotic cells, or that is effective in nonspecifically inhibiting a virus titer increase or in nonspecifically reducing a virus titer level in a eukaryotic or mammalian host system.

An effective therapeutic compound can refer to a compound that can reduce, prevent, or treat a state, disorder, disease, or condition when the compound is administered to a subject. In particular embodiments, an effective therapeutic compound can prevent, reduce, or treat the likelihood of a viral infection. An amount of the therapeutic compound that is effective will vary depending on the compound, the disease and its severity and the age, weight, physical condition and responsiveness of the subject to be treated. The exact dose and formulation will depend on the purpose of the treatment and can be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). In certain cases, “therapeutically effective amount” is used to mean an amount or dose sufficient to modulate, e.g., increase or decrease a desired activity e.g., by 10%, by 50%, or by 90%. Generally, a therapeutically effective amount is sufficient to cause an improvement in a clinically significant condition in the host following a therapeutic regimen involving one or more therapeutic compounds. The concentration or amount of the compound depends on the desired dosage and administration regimen. The effective amounts of compounds containing active agents include doses that partially or completely achieve the desired therapeutic, prophylactic, and/or biological effect. The actual amount effective for a particular application depends on the condition being treated and the route of administration.

Resistance Analysis of Therapeutics. The systems and methods of the present disclosure can be used to assess resistance to therapeutic compounds caused by mutations of a given protein represented in the deep mutational scanning libraries that result in reduced phenotypic susceptibility to a given antiviral therapeutic compound. In particular embodiments, in vitro resistance analysis studies can assess the potential barrier of a target virus to develop reduced susceptibility (i.e., resistance) to a therapeutic compound and to help in designing clinical studies. Virus resistant to a given therapeutic compound can be selected in cell culture, and the selection can provide a genetic threshold for resistance development. In particular embodiments, a therapeutic compound with a low genetic threshold may select for resistance with only one or two mutations. In contrast, a therapeutic compound with a high genetic threshold may require multiple mutations to select for resistance. In particular embodiments, the development of resistance in vitro can be assessed over a concentration range of a therapeutic compound spanning the anticipated concentration of the therapeutic compound that will be used in vivo. Selection of variants resistant to a therapeutic compound can be repeated more than once (e.g., with different strains of wild-type, with resistant strains, under high and low selective pressure) to determine if the same or different patterns of resistance mutations develop, and to assess the relationship of therapeutic compound concentration to the genetic barrier to resistance.

As discussed above, determining the mutations that might contribute to reduced susceptibility to a therapeutic compound using the systems and methods of the present disclosure can include sequencing barcodes after linking a barcode to a particular variant in a deep mutational scanning library. Identifying resistance mutations by this genotypic analysis can be useful in predicting clinical outcomes and supporting the proposed mechanism of action of a therapeutic compound. In particular embodiments, the pattern of mutations leading to resistance of a therapeutic compound can be compared with the pattern of mutations of other therapeutic compounds in the same class. In particular embodiments, resistance pathways can be characterized in several genetic backgrounds (i.e., strains, subtypes, genotypes) and protein variants can be obtained throughout the selection process to identify the order in which multiple mutations appear.

Phenotypic analysis determines if mutant viruses have reduced susceptibility to a therapeutic compound. In particular embodiments, using the systems and methods of the present disclosure, phenotypic analysis is performed when virions including protein variants of a cell-stored deep mutational scanning library are selected for resistance to a therapeutic compound. In particular embodiments, phenotypic resistance can be scored, for example, by an EC₅₀ value. An EC₅₀ value can refer to an effective concentration of a therapeutic compound which induces a response halfway between the baseline and maximum after a specified exposure time. In particular embodiments, an EC₅₀ value can be used as a measure of a therapeutic compound's potency. EC₅₀ can be expressed in molar units (M), where 1 M is equivalent to 1 mol/L. The fold resistant change can be calculated as the EC₅₀ value of the protein of interest/EC₅₀ value of a reference protein. Phenotypic results can be determined with any standard virus assay (e.g., protein assay, viral RNA assay, polymerase assay, MTT cytotoxic assay, reporter expression). In particular embodiments, virus titer can be calculated as a function of the concentration of the therapeutic compound to obtain an EC₅₀ value. In particular embodiments, virus titer can be calculated by a plaque assay or focus forming assay. A plaque assay takes advantage of plaques that can arise through virus-mediated cell death within a monolayer of a cell culture when cells are infected with a cytopathic virus and typically requires plaques to grow until visible to the naked eye. The focus-forming assay can be used to titer non-cytopathic viruses. This assay usually relies on the detection of infected cells by immunostaining for viral antigen or via a genetically encoded fluorescent reporter. The shift in susceptibility (or fold resistant change) for a protein variant can be measured by determining the EC₅₀ value for the protein of interest and comparing it to the EC₅₀ value of a reference protein. In particular embodiments, a reference protein can be a counterpart viral protein (equivalent viral protein having the same function from the same virus or from a different virus) from a wild-type virus, from a well-characterized wild-type laboratory strain, from a parental virus, or from a baseline clinical isolate done under the same conditions and at the same time. In particular embodiments, a wild-type virus can be naturally occurring. In particular embodiments, a wild-type virus has no mutations that confer drug resistance. In particular embodiments, a parental virus can be a virus having a viral protein that did not undergo mutagenesis as described herein to create a cell-stored barcoded deep mutational scanning library of variants of the viral protein. In particular embodiments, a parental virus can be a wild type virus. A baseline clinical isolate includes an isolate from a subject being screened for inclusion in a clinical trial or an isolate from a subject in a clinical trial before treatment in the trial has begun. The use of the EC₅₀ value for determining shifts in susceptibility can offer greater precision than an EC₉₀ or EC₉₅ value. The utility of a phenotypic assay depends on its sensitivity (i.e., its ability to measure shifts in susceptibility (fold resistance change) in comparison to a reference). Calculating the fold resistant change (EC₅₀ value of protein of interest/EC₅₀ value of reference protein) allows for comparisons among phenotypic assays.

A viral protein may develop mutations that lead to reduced susceptibility to one antiviral therapeutic compound and can result in decreased or loss of susceptibility to other antiviral therapeutic compounds in the same therapeutic compound class. This observation is referred to as cross-resistance. Cross-resistance is not necessarily reciprocal, so it is important to evaluate both possibilities. For example, if virus X is resistant to drug A and drug B, and virus Y is also resistant to drug A, virus Y may still be sensitive to drug B. In particular embodiments, the effectiveness of a therapeutic compound against viruses resistant to other approved therapeutic compounds in the same class and the effectiveness of approved therapeutic compounds belonging to a given class against viruses resistant to a therapeutic compound belonging to that same class can be evaluated by phenotypic analyses. In particular embodiments, cross-resistance can be analyzed between therapeutic classes in instances where more than one therapeutic compound class targets a single protein or protein complex (e.g., nucleoside reverse transcriptase inhibitors (NRTIs) and non-nucleoside reverse transcriptase inhibitors (NNRTIs), which both target the HIV-encoded reverse transcriptase). Variant viral proteins representative of the breadth of diverse mutations and combinations of mutations known to confer reduced susceptibility to therapeutic compounds in the same class can be tested for phenotypic susceptibility to a new therapeutic compound belonging to that same class.

(vii) Kits. Combinations of elements of the deep mutational scanning libraries disclosed herein can be provided as kits. Kits of the present disclosure can include: a landing pad sequence; a viral (e.g., lentiviral) vector for insertion into a landing pad; helper plasmids; and one or more cell lines. In particular embodiments, the viral vector includes a barcode. In particular embodiments, the viral vector includes sequences to facilitate sequencing. In particular embodiments, the viral vector includes a reporter gene. In particular embodiments, the viral vector encodes a protein of interest. In particular embodiments, the viral vector includes an integrase binding site. In particular embodiments, the viral vector includes all or a portion of a viral genome. In particular embodiments, the viral vector includes a promoter. In particular embodiments, the viral vector includes a selection marker. In particular embodiments, the viral vector includes a 5′LTR and/or a 3′LTR. In particular embodiments, the viral vector includes an insulator. In particular embodiments, the landing pad includes a promoter. In particular embodiments, the landing pad includes an integrase attachment site. In particular embodiments, the landing pad includes a promoter, selection marker, insulator, and/or a portion of a viral genome. In particular embodiments, the helper plasmids express Gag, Pol, Tat, and Rev. In particular embodiments, the one or more cell lines include HEK293T. In particular embodiments kits can include one or more cell-stored libraries as disclosed herein and/or one or more producer cells.

Kits can include further instructions for using the kit, for example, instructions regarding cloning of variant sequences into the viral vector, transfection of plasmids, selection of cells expressing a reporter protein, and propagation of the cell-stored library. The instructions can be in the form of printed instructions provided within the kit or the instructions can be printed on a portion of the kit itself. Instructions may be in the form of a sheet, pamphlet, brochure, CD-Rom, or computer-readable device, or can provide directions to instructions at a remote location, such as a website. In particular embodiments, kits can also include laboratory supplies needed to use the kit effectively, such as cell culture media, buffers, enzymes, sterile plates, sterile flasks, pipettes, gloves, and the like. Variations in contents of any of the kits described herein can be made.

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

(viii) Exemplary Embodiments.
1. A mammalian cell including a single copy of a viral vector integrated into a landing pad at a genomic locus of the mammalian cell, wherein the viral vector includes a first integrase binding site, a barcode, and a sequence encoding a protein of interest.
2. The mammalian cell of embodiment 1, wherein the genomic locus is at Adeno-associated virus site 1 (AAVS1).
3. The mammalian cell of embodiments 1 or 2, wherein the first integrase binding site includes a Bxb1 attB recombination site.
4. The mammalian cell of embodiment 3, wherein the Bxb1 attB recombination site includes: the sequence as set forth in SEQ ID NOs: 9 or 10 or a sequence having at least 90% sequence identity to the sequence set forth in SEQ ID NOs: 9 or 10.
5. The mammalian cell of any of embodiments 1-4, wherein the landing pad includes a second integrase binding site complementary to the first integrase binding site within the viral vector.
6. The mammalian cell of embodiment 5, wherein the second integrase binding site includes a Bxb1 attP recombination site.
7. The mammalian cell of embodiment 6, wherein the Bxb1 attP recombination site includes: the sequence as set forth in SEQ ID NO: 12; or a sequence having at least 90% sequence identity to the sequence set forth in SEQ ID NO: 12.
8. The mammalian cell of any of embodiments 1-7, wherein the viral vector further includes a first promoter.
9. The mammalian cell of embodiment 8, wherein the first promoter includes a CMV promoter.
10. The mammalian cell of embodiment 9, wherein the CMV promoter includes the sequence as set forth in SEQ ID NO: 2 or a sequence having at least 90% sequence identity to the sequence set forth in SEQ ID NO: 2.
11. The mammalian cell of embodiment 8, wherein the first promoter includes a TRE3GS promoter.
12. The mammalian cell of embodiment 11, wherein the TRE3GS promoter includes the sequence as set forth in SEQ ID NO: 45 or a sequence having at least 90% sequence identity to the sequence set forth in SEQ ID NO: 45.
13. The mammalian cell of any of embodiments 1-12, wherein the viral vector further includes a first selection marker.
14. The mammalian cell of embodiment 13, wherein the first selection marker includes a gene encoding a fluorescent protein.
15. The mammalian cell of embodiment 14, wherein the fluorescent protein includes zsGreen or DsRed.
16. The mammalian cell of any of embodiments 13-15, wherein the landing pad includes a second promoter and the first selection marker is under the regulatory control of the second promoter.
17. The mammalian cell of embodiment 16, wherein the second promoter includes a TRE3GV promoter.
18. The mammalian cell of embodiment 17, wherein the TRE3GV promoter includes the sequence as set forth in SEQ ID NO: 44 or a sequence having at least 90% sequence identity to the sequence set forth in SEQ ID NO: 44.
19. The mammalian cell of any of embodiments 13-18, wherein the viral vector further includes a second selection marker.
20. The mammalian cell of embodiment 19, wherein the second selection marker includes an antibiotic resistance gene.
21. The mammalian cell of any of embodiments 1-20, wherein the viral vector further includes a 5′ LTR and a 3′ LTR.
22. The mammalian cell of any of embodiments 1-21, wherein the viral vector further includes an insulator.
23. The mammalian cell of embodiment 22, wherein the insulator includes a cHS4 insulator.
24. The mammalian cell of embodiment 23, wherein the cHS4 insulator includes the sequence as set forth in SEQ ID NO: 1 or a sequence having at least 90% sequence identity to the sequence set forth in SEQ ID NO: 1.
25. The mammalian cell of any of embodiments 1-24, wherein the protein of interest is a viral protein, a receptor, or a receptor ligand.
26. The mammalian cell of embodiment 25, wherein the viral protein is a viral entry protein.
27. The mammalian cell of embodiment 25, wherein the receptor is a T cell receptor or a B cell receptor.
28. The mammalian cell of embodiment 25, wherein the receptor ligand is a hormone.
29. The mammalian cell of any of embodiments 1-28, within a library of mammalian cells.
30. The mammalian cell of embodiment 29, wherein each cell within the library including a single copy of a viral vector integrated into a landing pad at a genomic locus of the mammalian cell, wherein the viral vector includes a first integrase binding site, a barcode, and a sequence encoding a protein of interest
31. A method of genetically engineering a mammalian cell including:
   transfecting the mammalian cell with a landing pad including a second integrase binding site; and
   transfecting the mammalian cell with a viral vector including a first integrase binding site, wherein the viral vector further includes a barcode and a sequence encoding a protein of interest.
32. A method of preparing a library of genetically engineered mammalian cells including:
   Transfecting a population of mammalian cells with a landing pad, the landing pad including a first integrase binding site, a selection marker, and a promoter;
   selecting for cells having the landing pad;
   transfecting the selected cells with a viral vector, the viral vector including a second integrase binding site, a first sequence encoding a selectable marker, a barcode, and a second sequence encoding a protein of interest; and
   selecting for cells expressing the selectable marker under regulatory control of the promoter.
33. The method of embodiment 32, wherein the first integrase binding site is a complementary recombination site of the second integrase binding site.
34. The method of embodiments 32 or 33, wherein the first integrase binding site is a Bxb1 attB recombination site and the second integrase binding site is a Bxb1 attP recombination site.
35. The method of embodiments 32 or 33, wherein the first integrase binding site is a Bxb1 attP recombination site and the second integrase binding site is a Bxb1 attB recombination site.
36. A method of producing viral particles including transfecting the mammalian cell of any of embodiments 1-30 with helper plasmids.
37. The method of embodiment 36, wherein the helper plasmids encode Rev, Pol, Env, and Gag.
38. The method of embodiments 36 or 37, wherein the helper plasmids encode vesicular stomatitis virus G (VSV-G).
39. The method of any of embodiments 36-38, further including culturing the mammalian cell under conditions suitable for mammalian cell to produce viral particles.
40. The method of any of embodiments 36-39, wherein the mammalian cell includes a HEK293T cell.
41. A library of mammalian cells, wherein cells in the library include a single copy of a viral vector integrated into a landing pad at a genomic locus of the mammalian cell, wherein the viral vector includes a barcode and a sequence encoding a protein of interest.
42. The library of mammalian cells of embodiment 41, wherein the genomic locus is at Adeno-associated virus site 1 (AAVS1).
43. The library of mammalian cells of embodiments 41 or 42, further including a first integrase binding site in the landing pad and a second integrase binding site in the viral vector.
44. The library of mammalian cells of embodiment 43, wherein the first integrase binding site includes a Bxb1 attB recombination site and the second integrase binding site includes Bxb1 attP recombination site.
45. The library of mammalian cells of embodiment 43, wherein the first integrase binding site includes Bxb1 attP recombination site and the second integrase binding site includes a Bxb1 attB recombination site.
46. The library of mammalian cells of embodiments 44 or 45, wherein the Bxb1 attB recombination site includes: the sequence as set forth in SEQ ID NOs: 9 or 10; or a sequence having at least 90% sequence identity to the sequence set forth in SEQ ID NOs: 9 or 10.
47. The library of mammalian cells of embodiments 44 or 45, wherein the Bxb1 attP recombination site includes: the sequence as set forth in SEQ ID NO: 12; or a sequence having at least 90% sequence identity to the sequence set forth in SEQ ID NO: 12.
48. The library of mammalian cells of any of embodiments 41-47, wherein the viral vector further includes a first promoter.
49. The library of mammalian cells of embodiment 48, wherein the first promoter includes a CMV promoter.
50. The library of mammalian cells of embodiment 49, wherein the CMV promoter includes the sequence as set forth in SEQ ID NO: 2 or a sequence having at least 90% sequence identity to the sequence set forth in SEQ ID NO: 2.
51. The library of mammalian cells of embodiment 48, wherein the first promoter includes a TRE3GS promoter.
52. The library of mammalian cells of embodiment 51, wherein the TRE3GS promoter includes the sequence as set forth in SEQ ID NO: 45 or a sequence having at least 90% sequence identity to the sequence set forth in SEQ ID NO: 45.
53. The library of mammalian cells of any of embodiments 41-52, wherein the viral vector further includes a first selection marker.
54. The library of mammalian cells of embodiment 53, wherein the first selection marker includes a gene encoding a fluorescent protein.
55. The library of mammalian cells of embodiment 53 or 54, wherein the landing pad includes a second promoter and the first selection marker is under the regulatory control of the second promoter.
56. The library of mammalian cells of embodiment 55, wherein the second promoter includes a TRE3GV promoter.
57. The library of mammalian cells of embodiment 56, wherein the TRE3GV promoter includes the sequence as set forth in SEQ ID NO: 44 or a sequence having at least 90% sequence identity to the sequence set forth in SEQ ID NO: 44.
58. The library of mammalian cells of any of embodiments 41-57, wherein the viral vector further includes a second selection marker.
59. The library of mammalian cells of embodiment 58, wherein the second selection marker includes an antibiotic resistance gene.
60. The library of mammalian cells of any of embodiments 41-59, wherein the viral vector further includes a 5′ LTR and a 3′ LTR.
61. The library of mammalian cells of any of embodiments 41-60, wherein the viral vector further includes an insulator.
62. The library of mammalian cells of embodiment 61, wherein the insulator includes a cHS4 insulator.
63. The library of mammalian cells of embodiment 62, wherein the cHS4 insulator includes the sequence as set forth in SEQ ID NO: 1 or a sequence having at least 90% sequence identity to the sequence set forth in SEQ ID NO: 1.
64. The library of mammalian cells of any of embodiments 41-63, wherein the protein of interest is a viral protein, a receptor, or a receptor ligand.
65. The library of mammalian cells of embodiment 64, wherein the viral protein is a viral entry protein.
66. The library of mammalian cells of embodiment 64, wherein the receptor is a T cell receptor or a B cell receptor.
67. The library of mammalian cells of embodiment 64, wherein the receptor ligand is a hormone.

(ix) Experimental Examples. Example 1. Pooled high-throughput screens (hts) in mammalian cells require the delivery of DNA elements, e.g., single guide RNA (sgRNAs), open reading frames (ORFs) and reporters. This delivery is usually accomplished via lentiviral vectors engineered to encode the desired library elements to test. As hts grow in scale and complexity, the number of library elements to be delivered to target cells has increased. For example, dual instead of single sgRNA lead to better efficacy. Replogle, J. M. et al. Maximizing CRISPRi efficacy and accessibility with dual-sgRNA libraries and optimal effectors. biorxiv.org/lookup/doi/10.1101/2022.07.13.499814 (2022) doi:10.1101/2022.07.13.499814 Other experiments require the use of a barcode as a proxy for detecting a given element. In these and other cases, unique coupling of the library elements throughout an experiment is critical to ensure accuracy. A key source of uncoupling of DNA elements is lentiviral production. Lentiviruses are pseudo-diploid, and during their production via transfection, multiple plasmids might be introduced in a cell, resulting in the co-packaging of chimeric genomes. These lentiviruses will then undergo template switching during reverse transcription, which unlinks library elements from each other and ultimately results in lower assay sensitivity. Hill et al., (2018). On the design of CRISPRbased single-cell molecular screens. Nature Methods, 15(4), 271-274. doi.org/10.1038/nmeth.4604

Two main strategies have emerged to mitigate the effects of template switching: redesign of vectors and alternative lentiviral packaging methods. To mitigate the effects of template switching that were pervasive in commonly used screening vectors, Hill et al redesigned such vectors to avoid barcodes and use the sgRNA for direct detection during sequencing. This design avoided template switching in 94% of instances. Hill et al., (2018). On the design of CRISPRbased single-cell molecular screens. Nature Methods, 15(4), 271-274. doi.org/10.1038/nmeth.4604

Similarly, Sack et al overcame the effects of template switching for a barcoded ORF library by systematically testing different configurations and distances between the ORF and barcode. Sack et al., (2016). Sources of Error in Mammalian Genetic Screens. G3 Genes|Genomes|Genetics, 6(9), 2781-2790. doi.org/10.1534/g3.116.030973 By decreasing the distance between the ORF and barcode to 94 bp, they successfully reduced template switching to 6% of instances. However, with these approaches library design is constrained to minimize both the length and homology between paired sequence elements, which for some applications is not possible. Since the effects of template switching are caused at the transfection stage, Feldman et al sought to mitigate it by introducing a heterologous carrier to be copackaged with the desired library. Feldman et al., (2018). Lentiviral co-packaging mitigates the effects of intermolecular recombination and multiple integrations in pooled genetic screens [Preprint]. Genomics. doi.org/10.1101/262121. This method reduced barcode swapping to 6%, and unlike the ones above, does not depend on a particular library design. However, titers are reduced by more than 100 fold, limiting its use to small scale libraries. Thus a practical, robust, and flexible approach to avoid the effects of template switching is lacking. Moreover, none of these approaches preserve linkage between the lentivirus and DNA elements that reside outside the lentiviral genome, which is critical for certain applications, such as pseudotyping with pooled viral entry proteins, capsids or receptor protein variants.

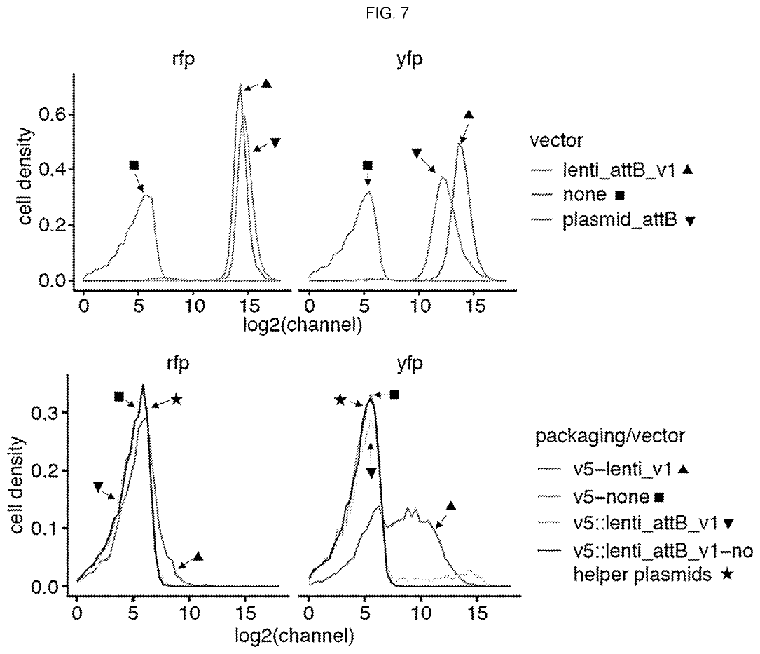

Landing pads enable the integration of a single event into a safe harbor locus in mammalian cells. Thus, it was reasoned that successfully integrating lentiviral vectors and subsequently rescuing lentiviruses could overcome the effects of template switching by ensuring a single library element is packaged in each lentivirus. As a proof of concept, a lentiviral vector expressing ZsGreen and DsRed in the lentiviral genome was engineered to encode an attB site (lenti_attB_v1), which upon cotransfection with a recombinase would recombine with an attP site pre-installed in the landing pad of HEK293T cells (cell line v5). An mCherry was encoded to be driven from the landing pad promoter upon integration. Integrants were then selected for and integration was assessed by monitoring fluorescence of the reporters by flow cytometry. The lentiviral vector integrated successfully, to the same extent as a nonlentiviral vector used as a positive control (plasmid_attB) (FIG. 7, top). Those cells (v5::lenti_attB_v1) were then used to package lentiviruses by transfecting helper plasmids (VSV-G and gag-pol-pro), or omitting them as a negative control (v5::lenti_attB_v1-no helper plasmids). As a positive control for packaging, the parent lentiviral vector without the attB site (lenti_v1) was used. As background, helper plasmids were transfected in cells with no lentiviral genome (v5—none) Two days later, the spent media was collected, filtered, and used to transduce cells. Successful packaging and transduction was assessed by flow cytometry. A proportion of target cells infected with spent media from v5::lenti_attB_v1, but not those from the negative controls, expressed ZsGreen (FIG. 7, bottom). This experiment demonstrated that lentiviral genomes can be 1) integrated into landing pads and 2) can be packaged into functional lentiviral particles. DsRed was not detected on cells transduced with either the parent or attB vectors, indicating that this result is independent of the disclosed landing pad approach.

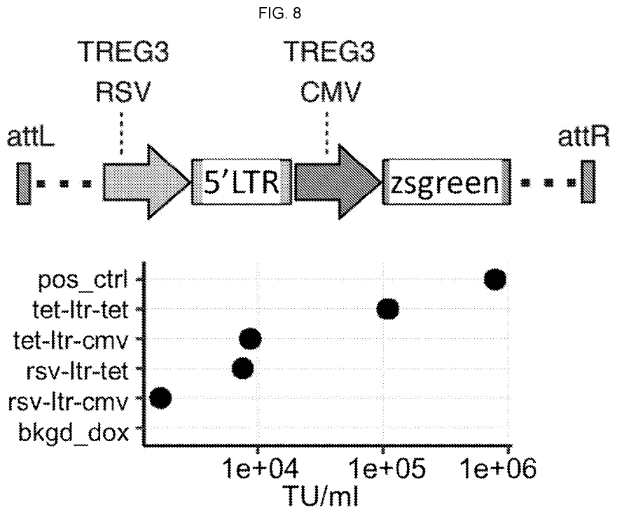

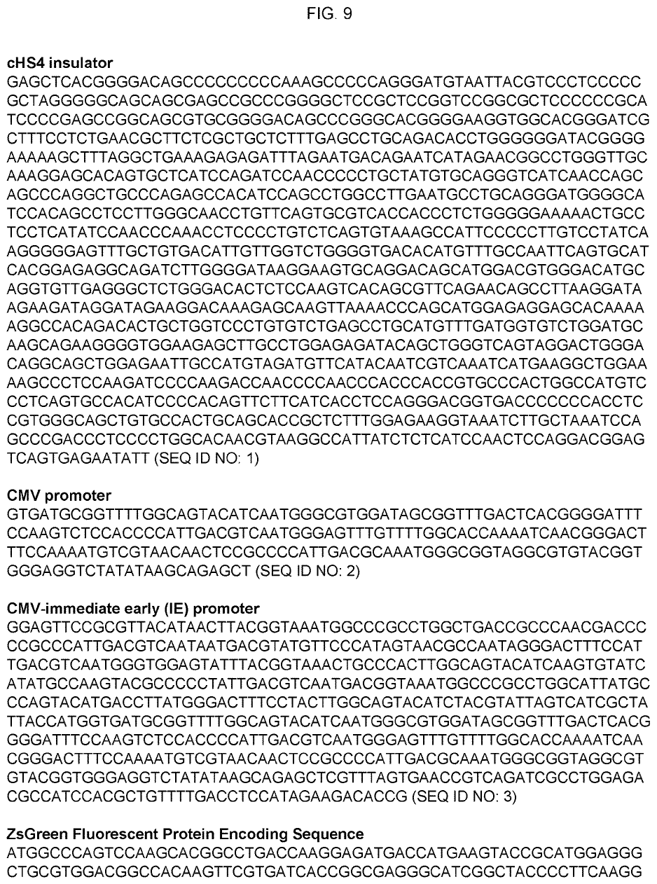

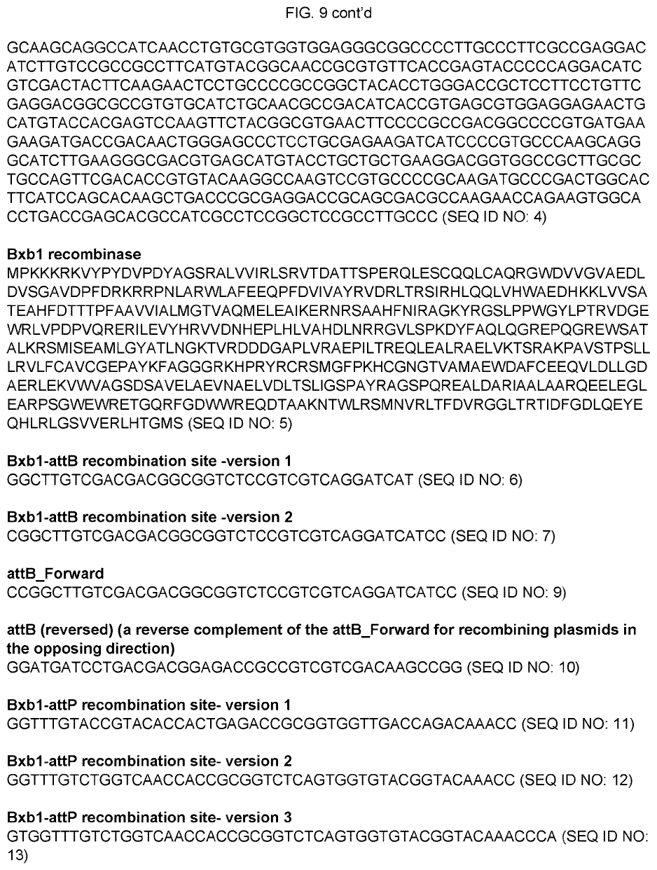

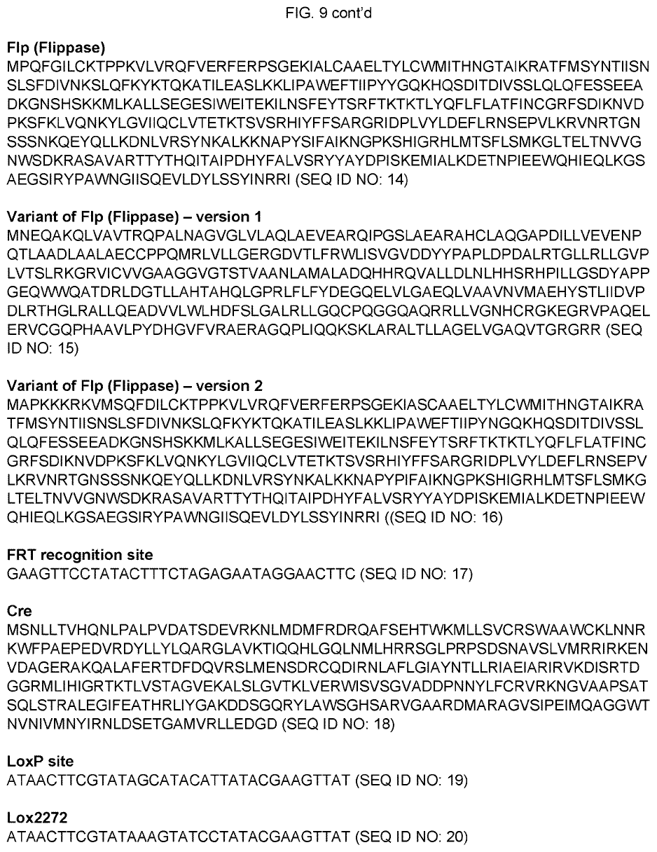

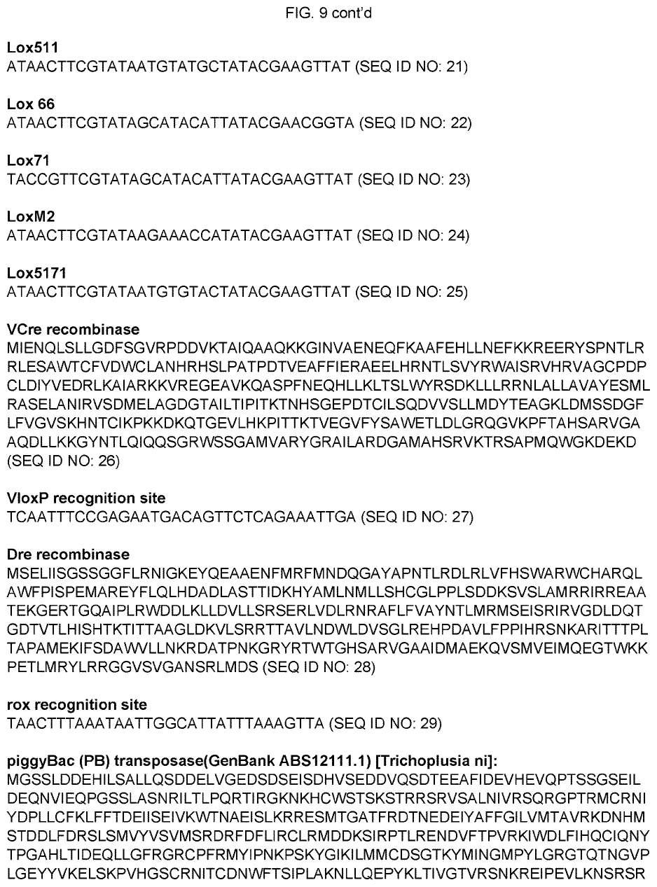

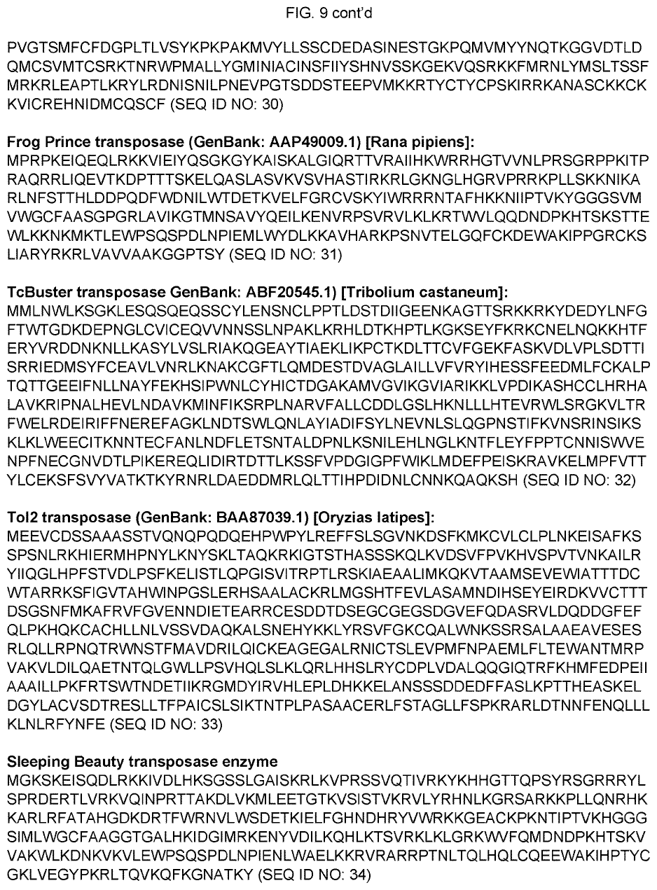

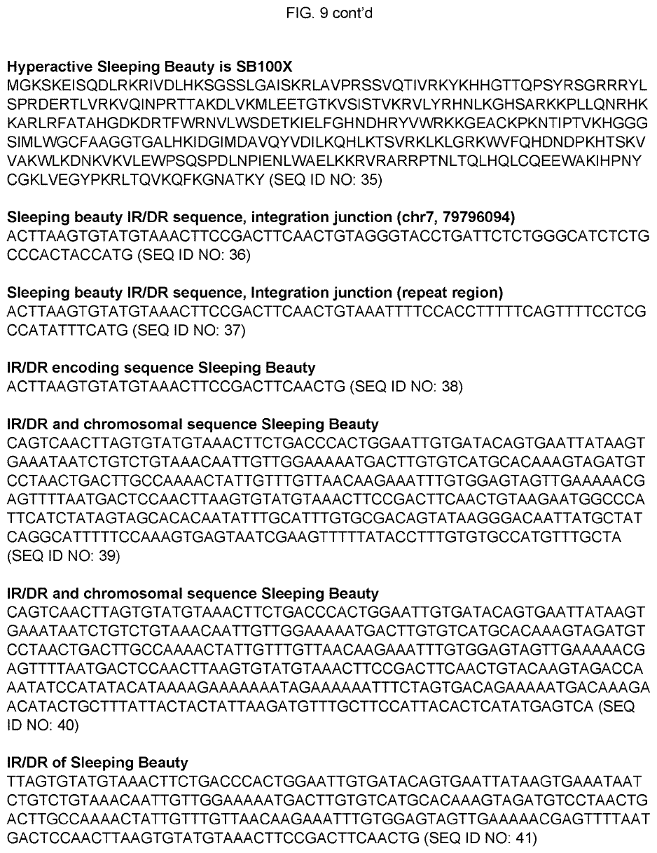

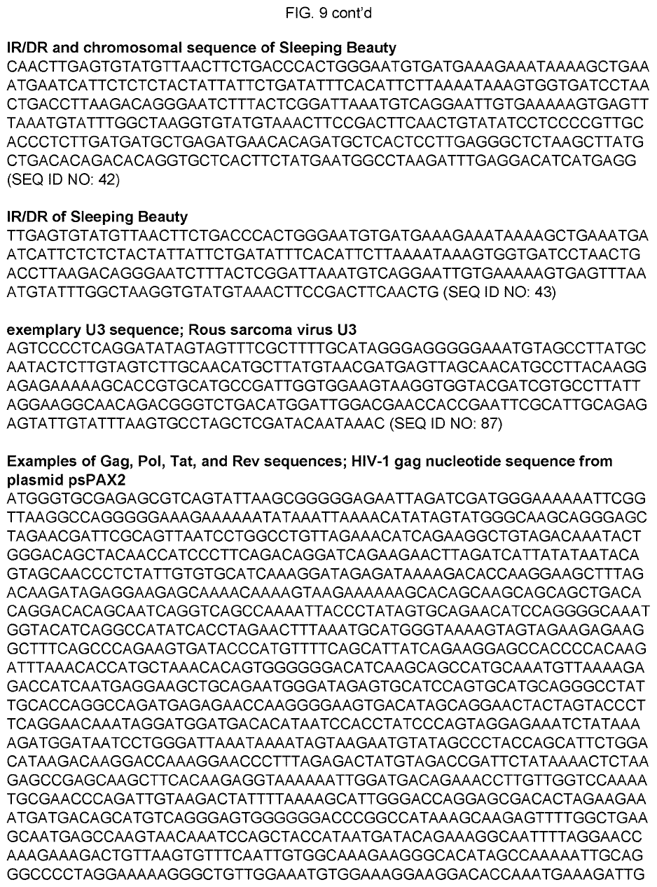

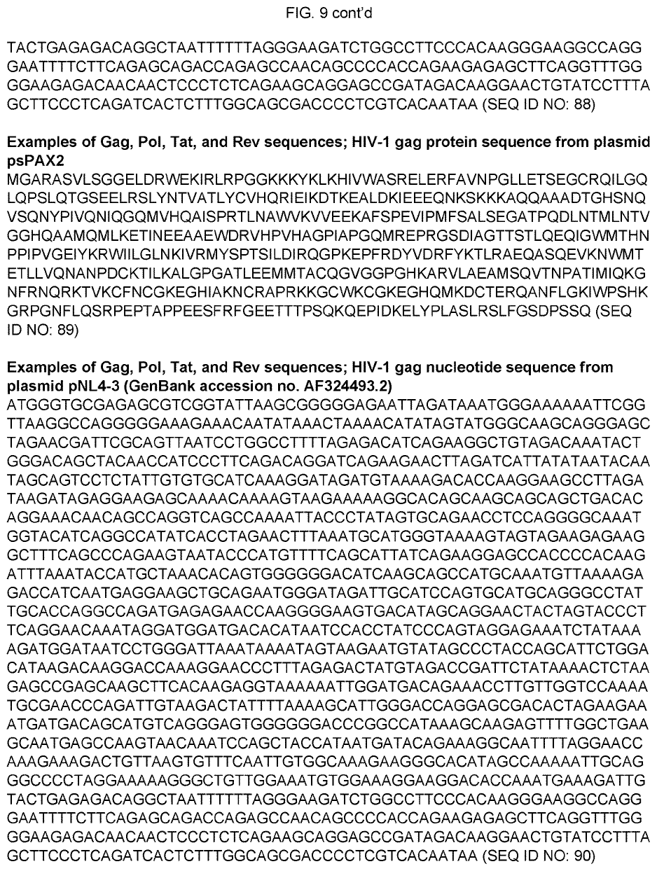

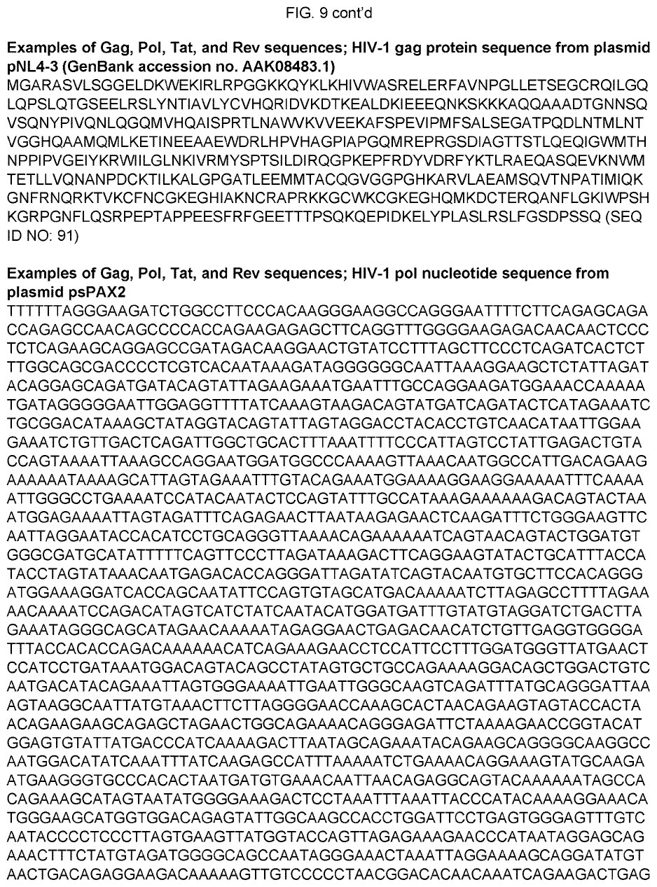

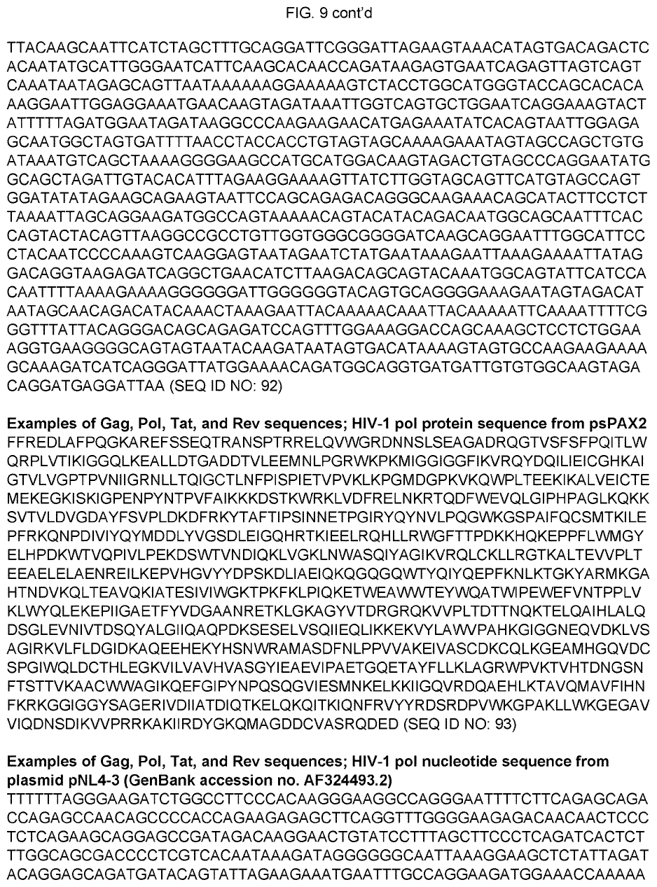

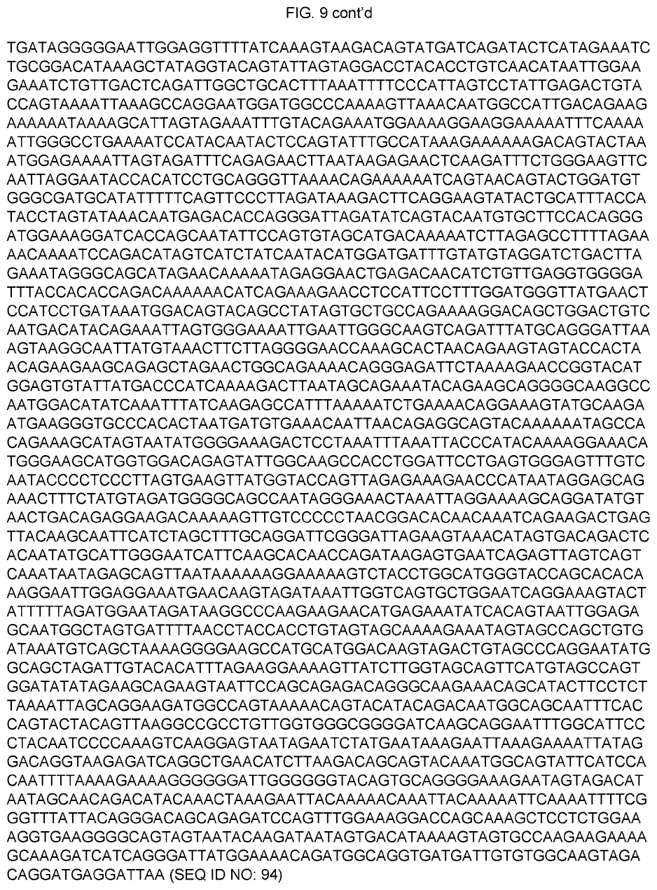

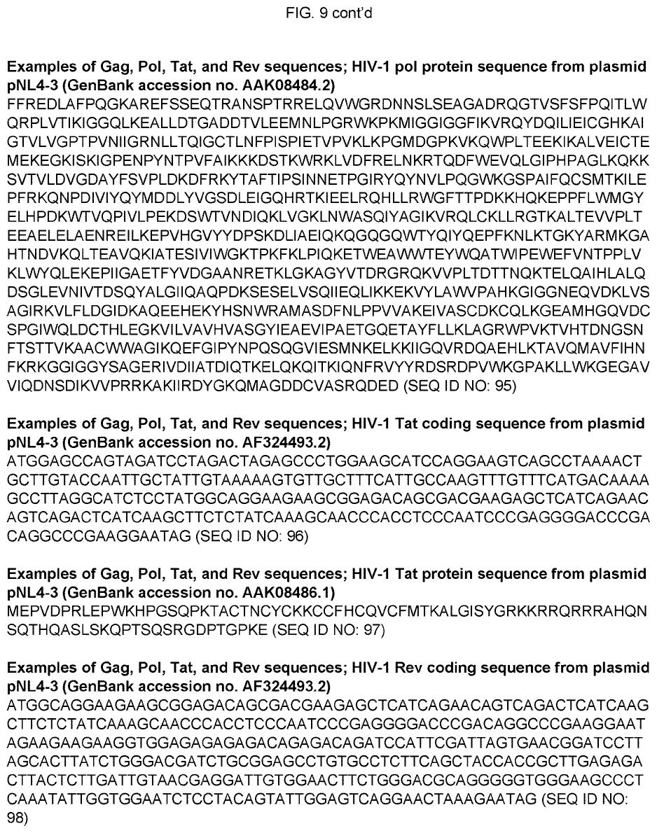

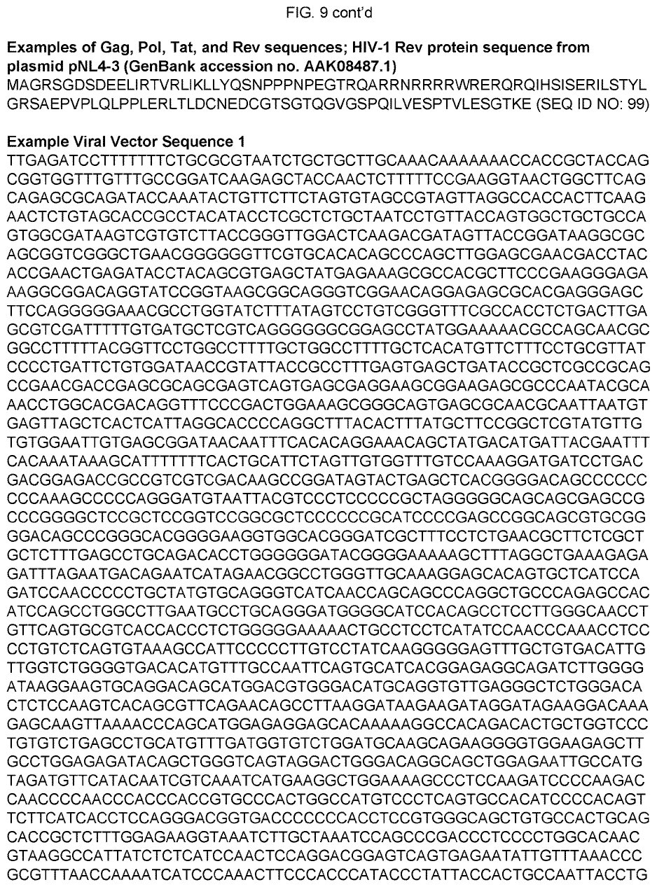

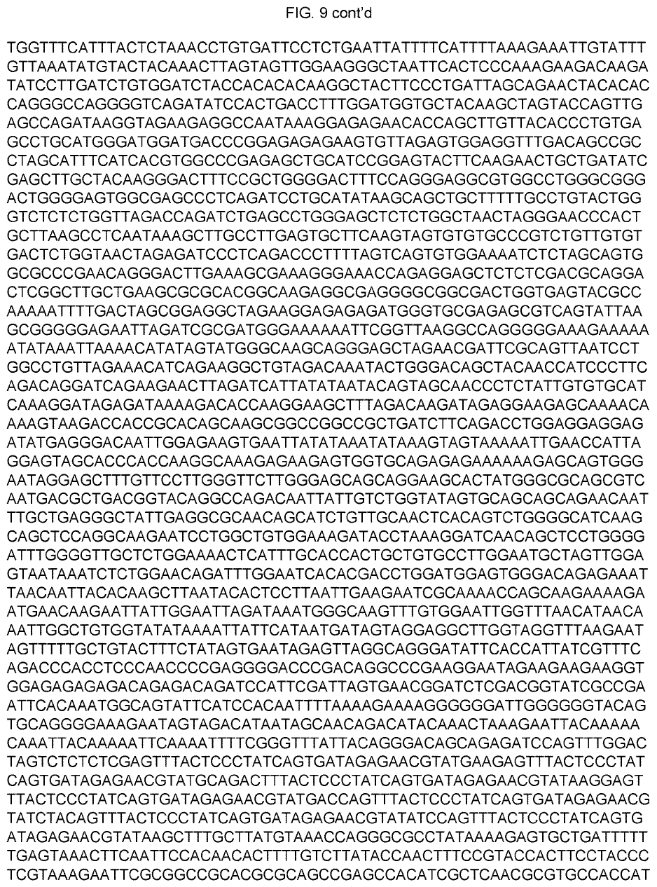

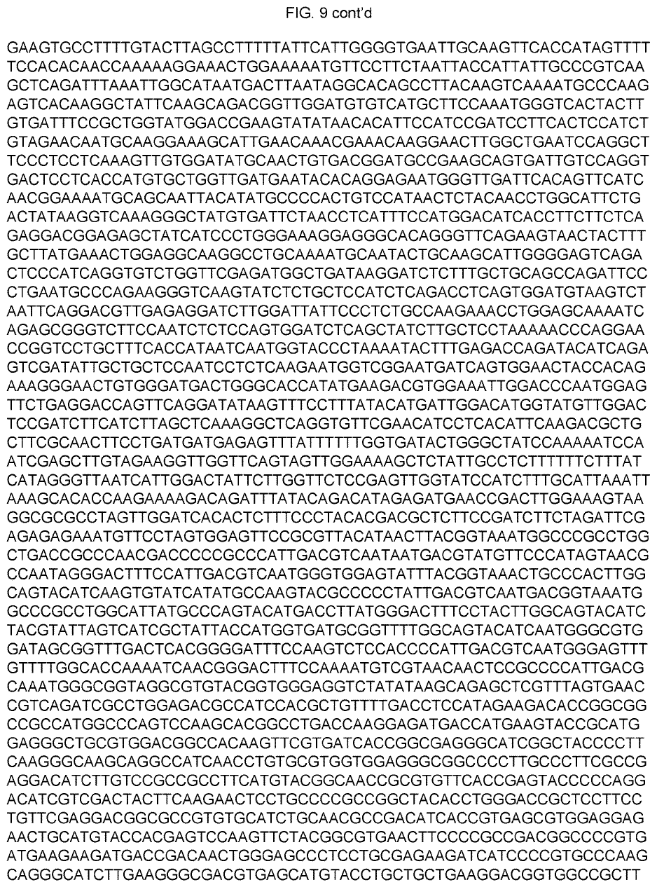

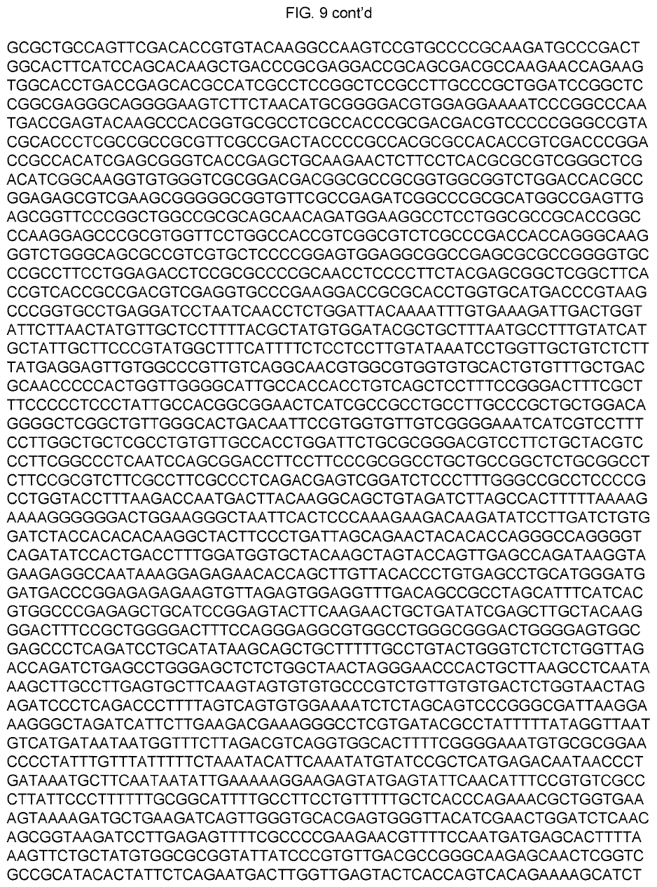

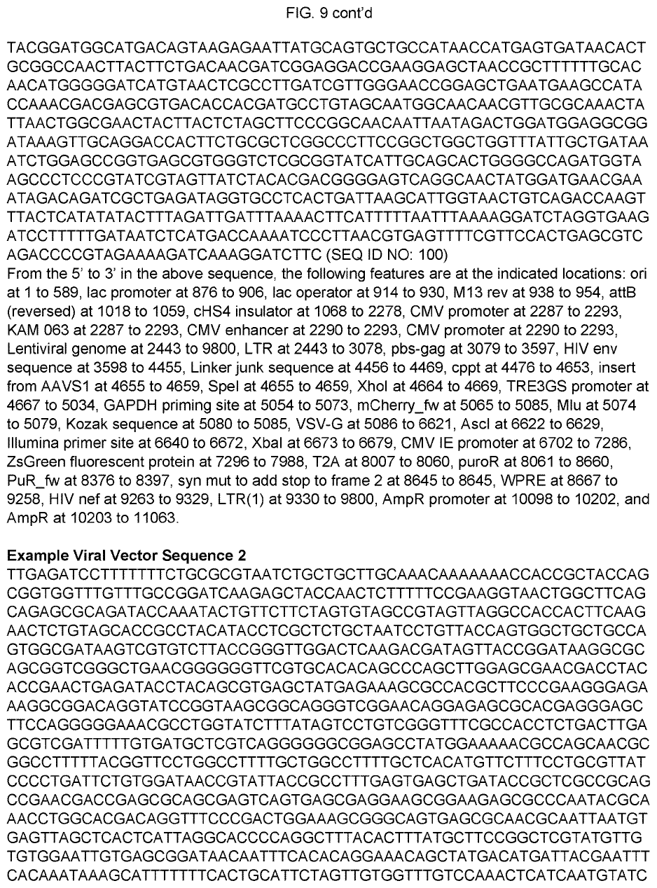

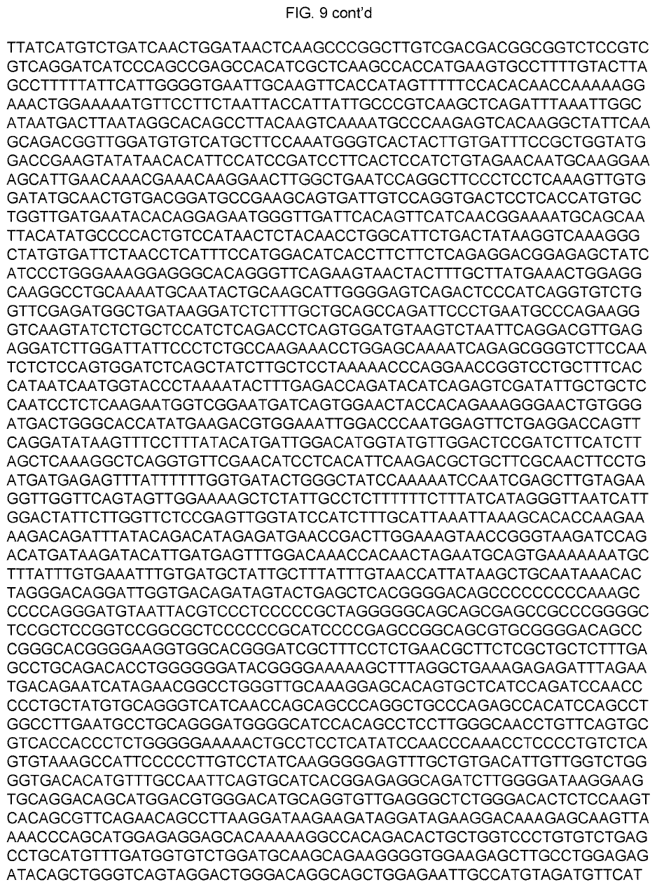

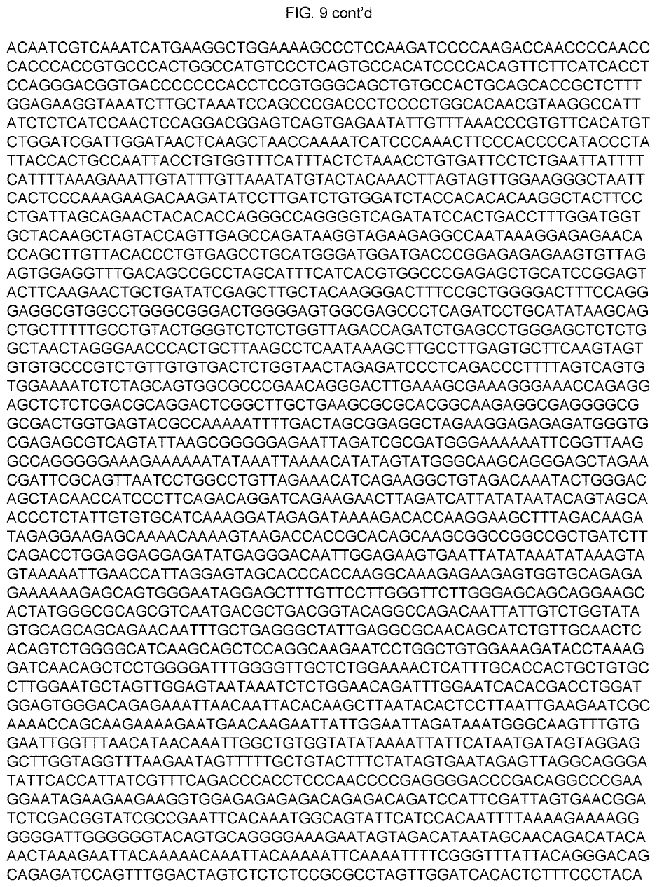

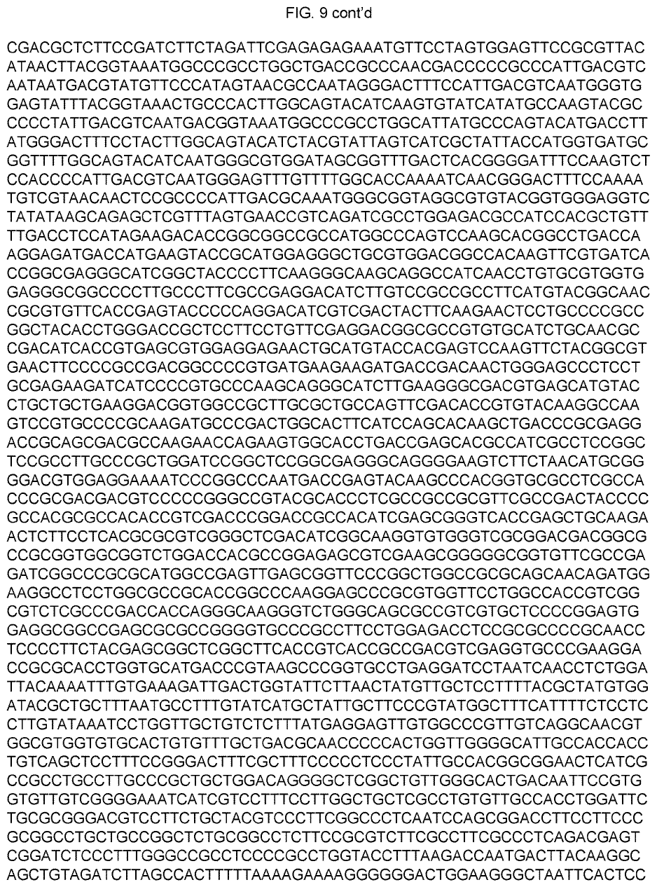

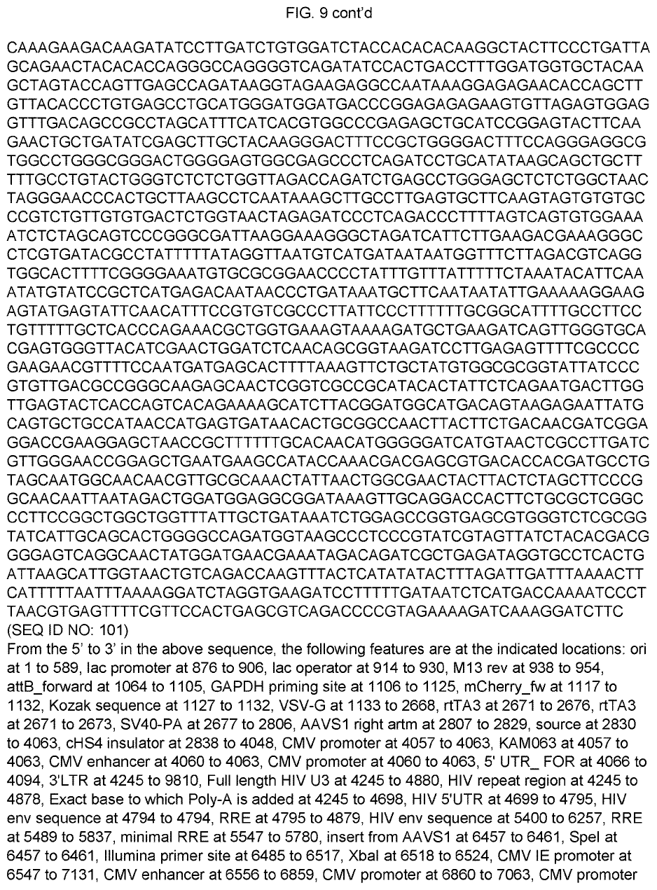

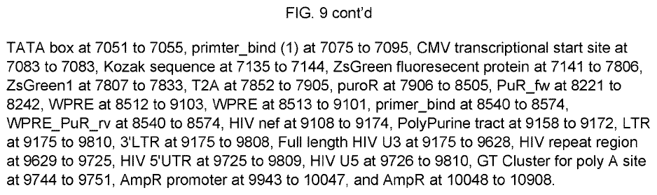

Given that the vector above was nonoptimal and titers were low, viral vector design was further optimized. Lentiviral vectors with distinct combinations of promoters driving 5′LTR expression as well as reporter expression within the lentiviral genome (FIG. 8) were engineered. These viral vectors were integrated into landing pad cells, selected and rescued by co-transfecting with helper plasmids, as above. Promoter combinations had a strong effect on titers, with a tre3g promoter in both locations performing optimally. These results suggest that optimizing the promoter identities and configurations might further improve titers.

(x) Closing Paragraphs. The nucleic acid and amino acid sequences provided herein are shown using letter abbreviations for nucleotide bases and amino acid residues, as defined in 37 C.F.R. § 1.831-1.835 and set forth in WIPO Standard ST.26 (implemented on Jul. 1, 2022). Only one strand of each nucleic acid sequence is shown, but the complementary strand is understood as included in embodiments where it would be appropriate.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (lie), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, lie, Phe, Met, and Trp; Group 11 (aliphatic): Gly, Ala, Val, Leu, and lie; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridize under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to maintain a genotype-phenotype link within a transfected cell line, as described herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; 19% of the stated value; ±18% of the stated value; 17% of the stated value; 16% of the stated value; ±15% of the stated value; 14% of the stated value; ±13% of the stated value; 12% of the stated value; 11% of the stated value; 10% of the stated value; 9% of the stated value; 8% of the stated value; 7% of the stated value; ±6% of the stated value; 5% of the stated value; 4% of the stated value; ±3% of the stated value; 2% of the stated value; or +1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A mammalian cell comprising a single copy of a viral vector integrated into a landing pad at a genomic locus of the mammalian cell, wherein the viral vector comprises a first integrase binding site, a barcode, and a sequence encoding a protein of interest.

2. The mammalian cell of claim 1, wherein the genomic locus is at Adeno-associated virus site 1 (AAVS1).

3. The mammalian cell of claim 1, wherein the first integrase binding site comprises a Bxb1 attB recombination site.

4. The mammalian cell of claim 3, wherein the Bxb1 attB recombination site comprises: the sequence as set forth in SEQ ID NOs: 9 or 10 or a sequence having at least 90% sequence identity to the sequence set forth in SEQ ID NOs: 9 or 10.

5. The mammalian cell of claim 1, wherein the landing pad comprises a second integrase binding site complementary to the first integrase binding site within the viral vector.

6. The mammalian cell of claim 5, wherein the second integrase binding site comprises a Bxb1 attP recombination site.

7. The mammalian cell of claim 6, wherein the Bxb1 attP recombination site comprises: the sequence as set forth in SEQ ID NO: 12; or a sequence having at least 90% sequence identity to the sequence set forth in SEQ ID NO: 12.

8. The mammalian cell of claim 1, wherein the viral vector further comprises a first promoter.

9. The mammalian cell of claim 8, wherein the first promoter comprises a CMV promoter.

10. The mammalian cell of claim 9, wherein the CMV promoter comprises the sequence as set forth in SEQ ID NO: 2 or a sequence having at least 90% sequence identity to the sequence set forth in SEQ ID NO: 2.

11. The mammalian cell of claim 8, wherein the first promoter comprises a TRE3GS promoter.

12. The mammalian cell of claim 11, wherein the TRE3GS promoter comprises the sequence as set forth in SEQ ID NO: 45 or a sequence having at least 90% sequence identity to the sequence set forth in SEQ ID NO: 45.

13. The mammalian cell of claim 1, wherein the viral vector further comprises a first selection marker.

14. The mammalian cell of claim 13, wherein the first selection marker comprises a gene encoding a fluorescent protein.

15. The mammalian cell of claim 14, wherein the fluorescent protein comprises zsGreen or DsRed.

16. The mammalian cell of claim 13, wherein the landing pad comprises a second promoter and the first selection marker is under regulatory control of the second promoter.

17. The mammalian cell of claim 16, wherein the second promoter comprises a TRE3GV promoter.

18. The mammalian cell of claim 17, wherein the TRE3GV promoter comprises the sequence as set forth in SEQ ID NO: 44 or a sequence having at least 90% sequence identity to the sequence set forth in SEQ ID NO: 44.

19. The mammalian cell of claim 13, wherein the viral vector further comprises a second selection marker.

20. The mammalian cell of claim 19, wherein the second selection marker comprises an antibiotic resistance gene.

21. The mammalian cell of claim 1, wherein the viral vector further comprises a 5′ LTR and a 3′ LTR.

22. The mammalian cell of claim 1, wherein the viral vector further comprises an insulator.

23. The mammalian cell of claim 22, wherein the insulator comprises a cHS4 insulator.

24. The mammalian cell of claim 23, wherein the cHS4 insulator comprises the sequence as set forth in SEQ ID NO: 1 or a sequence having at least 90% sequence identity to the sequence set forth in SEQ ID NO: 1.

25. The mammalian cell of claim 1, wherein the protein of interest is a viral protein, a receptor, or a receptor ligand.

26. The mammalian cell of claim 25, wherein the viral protein is a viral entry protein.

27. The mammalian cell of claim 25, wherein the receptor is a T cell receptor or a B cell receptor.

28. The mammalian cell of claim 25, wherein the receptor ligand is a hormone.

29. The mammalian cell of claim 1, within a library of mammalian cells.

30. The mammalian cell of claim 29, wherein each cell within the library comprises a single copy of a viral vector integrated into a landing pad at a genomic locus of the mammalian cell, wherein the viral vector comprises a first integrase binding site, a barcode, and a sequence encoding a protein of interest.

31. A method of genetically engineering a mammalian cell comprising:
transfecting the mammalian cell with a landing pad comprising a second integrase binding site; and
transfecting the mammalian cell with a viral vector comprising a first integrase binding site, wherein the viral vector further comprises a barcode and a sequence encoding a protein of interest.

32. A method of preparing a library of genetically engineered mammalian cells comprising:
transfecting a population of mammalian cells with a landing pad, the landing pad comprising a first integrase binding site, a selection marker, and a promoter;
selecting for cells having the landing pad;
transfecting the selected cells with a viral vector, the viral vector comprising a second integrase binding site, a first sequence encoding a selectable marker, a barcode, and a second sequence encoding a protein of interest; and
selecting for cells expressing the selectable marker under regulatory control of the promoter.

33. The method of claim 32, wherein the first integrase binding site is a complementary recombination site of the second integrase binding site.

34. The method of claim 32, wherein the first integrase binding site is a Bxb1 attB recombination site and the second integrase binding site is a Bxb1 attP recombination site.

35. The method of claim 32, wherein the first integrase binding site is a Bxb1 attP recombination site and the second integrase binding site is a Bxb1 attB recombination site.

36. A method of producing viral particles comprising transfecting the mammalian cell of claim 1 with helper plasmids.

37. The method of claim 36, wherein the helper plasmids encode Rev, Pol, Env, and Gag.

38. The method of claim 36, wherein the helper plasmids encode vesicular stomatitis virus G (VSV-G).

39. The method of claim 36, further comprising culturing the mammalian cell under conditions suitable for the mammalian cell to produce viral particles.

40. The method of claim 36, wherein the mammalian cell comprises a HEK293T cell.

41. A library of mammalian cells, wherein cells in the library comprise a single copy of a viral vector integrated into a landing pad at a genomic locus of the mammalian cell, wherein the viral vector comprises a barcode and a sequence encoding a protein of interest.

42. The library of mammalian cells of claim 41, wherein the genomic locus is at Adeno-associated virus site 1 (AAVS1).

43. The library of mammalian cells of claim 41, further comprising a first integrase binding site in the landing pad and a second integrase binding site in the viral vector.

44. The library of mammalian cells of claim 43, wherein the first integrase binding site comprises a Bxb1 attB recombination site and the second integrase binding site comprises Bxb1 attP recombination site.

45. The library of mammalian cells of claim 43, wherein the first integrase binding site comprises Bxb1 attP recombination site and the second integrase binding site comprises a Bxb1 attB recombination site.

46. The library of mammalian cells of claim 44 or 45, wherein the Bxb1 attB recombination site comprises: the sequence as set forth in SEQ ID NOs: 9 or 10; or a sequence having at least 90% sequence identity to the sequence set forth in SEQ ID NOs: 9 or 10.

47. The library of mammalian cells of claim 44 or 45, wherein the Bxb1 attP recombination site comprises: the sequence as set forth in SEQ ID NO: 12; or a sequence having at least 90% sequence identity to the sequence set forth in SEQ ID NO: 12.

48. The library of mammalian cells of claim 41, wherein the viral vector further comprises a first promoter.

49. The library of mammalian cells of claim 48, wherein the first promoter comprises a CMV promoter.

50. The library of mammalian cells of claim 49, wherein the CMV promoter comprises the sequence as set forth in SEQ ID NO: 2 or a sequence having at least 90% sequence identity to the sequence set forth in SEQ ID NO: 2.

51. The library of mammalian cells of claim 48, wherein the first promoter comprises a TRE3GS promoter.

52. The library of mammalian cells of claim 51, wherein the TRE3GS promoter comprises the sequence as set forth in SEQ ID NO: 45 or a sequence having at least 90% sequence identity to the sequence set forth in SEQ ID NO: 45.

53. The library of mammalian cells of claim 41, wherein the viral vector further comprises a first selection marker.

54. The library of mammalian cells of claim 53, wherein the first selection marker comprises a gene encoding a fluorescent protein.

55. The library of mammalian cells of claim 53, wherein the landing pad comprises a second promoter and the first selection marker is under the regulatory control of the second promoter.

56. The library of mammalian cells of claim 55, wherein the second promoter comprises a TRE3GV promoter.

57. The library of mammalian cells of claim 56, wherein the TRE3GV promoter comprises the sequence as set forth in SEQ ID NO: 44 or a sequence having at least 90% sequence identity to the sequence set forth in SEQ ID NO: 44.

58. The library of mammalian cells of claim 41, wherein the viral vector further comprises a second selection marker.

59. The library of mammalian cells of claim 58, wherein the second selection marker comprises an antibiotic resistance gene.

60. The library of mammalian cells of claim 41, wherein the viral vector further comprises a 5′ LTR and a 3′ LTR.

61. The library of mammalian cells of claim 41, wherein the viral vector further comprises an insulator.

62. The library of mammalian cells of claim 61, wherein the insulator comprises a cHS4 insulator.

63. The library of mammalian cells of claim 62, wherein the cHS4 insulator comprises the sequence as set forth in SEQ ID NO: 1 or a sequence having at least 90% sequence identity to the sequence set forth in SEQ ID NO: 1.

64. The library of mammalian cells of claim 41, wherein the protein of interest is a viral protein, a receptor, or a receptor ligand.

65. The library of mammalian cells of claim 64, wherein the viral protein is a viral entry protein.

66. The library of mammalian cells of claim 64, wherein the receptor is a T cell receptor or a B cell receptor.

67. The library of mammalian cells of claim 64, wherein the receptor ligand is a hormone.
